SEQUENCE LISTING (4 sequences)

SEQ ID NO: 1 - DNA, 1577 bp, Unknown
  Organism is identified as KPF0019; it has been deposited with the ATCC, accession number SD5361
atgttcctcttgatggttcccttgtttagctacctggctgctgcctctctgtgagcactcctttttttacctttctttccctatcttaaagtcaaaatactaaccatctcatcagacgggtgctctccccacaaccagtcccatgcgacacccccgagcttggttaccaatgtgatcaaaagaccacccacacatggggtcaatactcgcccttcttctccgtcccatcggagatctccccctccgtcccctcaggctgccgccttaccttcgcccaagttctctcccgtcacggcgcccgcttcccaaccgccggcaaggccgccgccatctctgccgtcctgaccaaaattaaaacctccgccacctggtacgcccccgacttcgagttcatcaaagactacaactacgtcctcggcgtagaccacctcaccgccttcggcgagcaagagatggtcaactccggcatcaaattctaccagcgctacgcttccctcctccgggactacaccgacccagaatcgctccccttcgtccgcgcctcggggcaggagcgcgtcattgcctcagccaaaaacttcacaacaggcttttactccgccctcctcgctgataagaacccaccgccttcctccctcccgcttccccgccaggaaatggtcatcatttccgaatcgcccacggccaataacaccatgcaccacggcctctgccgcgccttcgaggattccaccaccggcgactcggtccaggcaaccttcatagccgctaacttcccgcctatcaccgcgcgcttgaatgcacagggtttcaaaggcgttgaactttctgacacggacgtgctctcgctcatggatttgtgtccgtttgataccgtcgcttacccgccctcctctctcaccaccttgtcctctccctccaggggatccaagctgctatcccccttctgctcccttttttcggcacaagactttacggtatacgactatctccaatccctcggcaagttctacgggtacggccccggtaactttctgggtgccacgcaaggagtggggtacgtgaacgagcttttggctcgcctcacccgttccccggtggtggataacacgacgactaattccacgctggatgggaacgaggagacgttcccgttgacgaagaataggacggtgtttgcggatttcagtcatgataatgatatgatggggatcttgactgctttgaggctcttcgagactgtagaagggatggacaatacgacaataccaaaagggtatggaagcacgggggatgagccaggactgaaagagagggagggggtgtttaaggtggggtgggcggtgccgtttgcggggagagtgtattttgagaagatggtttgtgatggtgacggggacggggagattgaccaaggagaagaggaacaagagttggtgaggatcttggttaatgatagagtggttaaactaaatgggtgtgaggcggatgagttggggagatgcaagttgggtaagtttgtggaaagtatggaatttgctaggaggggtggggattgggacaagtgttttgcttag

SEQ ID NO: 2 - protein, 503 aa, Unknown
  Organism is identified as KPF0019; it has been deposited with the ATCC, assigned accession number SD5361
MFLLMVPLFSYLAAASLRVLSPQPVPCDTPELGYQCDQKTTHTWGQYSPFFSVPSEISPSVPSGCRLTFAQVLSRHGARFPTAGKAAAISAVLTKIKTSATWYAPDFEFIKDYNYVLGVDHLTAFGEQEMVNSGIKFYQRYASLLRDYTDPESLPFVRASGQERVIASAKNFTTGFYSALLADKNPPPSSLPLPRQEMVIISESPTANNTMHHGLCRAFEDSTTGDSVQATFIAANFPPITARLNAQGFKGVELSDTDVLSLMDLCPFDTVAYPPSSLTTLSSPSRGSKLLSPFCSLFTAQDFTVYDYLQSLGKFYGYGPGNFLGATQGVGYVNELLARLTRSPVVDNTTTNSTLDGNEETFPLTKNRTVFADFSHDNDMMGILTALRLFETVEGMDNTTIPKGYGSTGDEPGLKEREGVFKVGWAVPFAGRVYFEKMVCDGDGDGEIDQGEEEQELVRILVNDRVVKLNGCEADELGRCKLGKFVESMEFARRGGDWDKCFA

SEQ ID NO: 3 - DNA, 1477 bp, Unknown
  Organism is identified as KPF0019; it has been deposited with the ATCC, assigned accession number SD5361
tccctcgagaagagatctccacaaccagttccatgtgatactccagagttaggttatcaatgtgaccaaaaaactactcatacttggggactttactcaccatacttctctgttgcttctgagatttcaccttcagttccaaagggatgtaggttaacttttgcacaagttttatctaggcatggagctagattccctactgctggagctgctgctgctatatctgctgttattacaaagattaagacatctgctacatggtacgcaccagactacgagttcattaaagattacaactatgttttgggtgttgaccatttaacagcttttggtgaacaagaaatggtcaactcaggaataaagttttaccagaggtatgcttcattgttgagaaactacacagatcctgaatcattgcctttcattagagcatcaggtcaagaaagagtcattgcatctgcaaagaacttcactactggtttctactctgctttgttggctgacaaaaatcctccaccttcttctttgccattgcctagacaagagaatgtcattatatcagagtctccaacagcaaacaatacaatgcaccacggtttgtgtagagcttttgaagattcaacaactggagattctgttcaggctactttcattgctgctaattttcctcctattactgcaaggttgaacgctcagggtttcaaaggagttgaattatctgatacagacgttttgtcattgatggatttgtgcccatttgacacagttgcataccctccatcttcattgactactttatcatcaccttcaagaggttcaaagttgttatctccattctgctctttgttcacagcacaagacttcattgtttacgactacttgcaatctttagaaaaattttatggttatggtccaggaaacttcttgggagctacacaaggagtcggatacgttaatgagttgttagctaggttaactcattcaccagtcgttgacaacacaactactaattcaactttggatggaaacgaggaaacatttccattgacaaagaacagaactgtctttgcagatttctcacatgacaatactatgatgggaatattaactgctttgagattgttcgaaactgtcaagggtatggataacacaacaattccaaaaggatacggatctacaggagacgaacctggattgaaggaaagagagggtgttttttctgttggatgggcagtcccttttgctggtagggtttactttgagaaaatggtttgtgatggagacggagatggtgaaattgatcaaggtgaggaggagcaagagttggtcagaattttggtcaatgacagagtcgtcaagttgaacggatgtgctgctgatgagttaggtagatgcaaattgggaaagtttgtcgagtcaatggaatttgctagaaggggtggtgattgggacaagtgtttcgcttaagtcgacgcgt

SEQ ID NO: 4 - protein, 483 aa, Unknown
  Organism is identified as KPF0019; it has been deposited with the ATCC, assigned accession number SD5361
SPQPVPCDTPELGYQCDQKTTHTWGLYSPYFSVASEISPSVPKGCRLTFAQVLSRHGARFPTAGAAAAISAVITKIKTSATWYAPDYEFIKDYNYVLGVDHLTAFGEQEMVNSGIKFYQRYASLLRNYTDPESLPFIRASGQERVIASAKNFTTGFYSALLADKNPPPSSLPLPRQENVIISESPTANNTMHHGLCRAFEDSTTGDSVQATFIAANFPPITARLNAQGFKGVELSDTDVLSLMDLCPFDTVAYPPSSLTTLSSPSRGSKLLSPFCSLFTAQDFIVYDYLQSLEKFYGYGPGNFLGATQGVGYVNELLARLTHSPVVDNTTTNSTLDGNEETFPLTKNRTVFADFSHDNTMMGILTALRLFETVKGMDNTTIPKGYGSTGDEPGLKEREGVFSVGWAVPFAGRVYFEKMVCDGDGDGEIDQGEEEQELVRILVNDRVVKLNGCAADELGRCKLGKFVESMEFARRGGDWDKCFA

(19) United States
(12) Patent Application Publication
(10) Pub. No.: US 2007/0184521 A1
(43) Pub. Date: Aug. 9, 2007

(54) Novel phytase and gene

(76) Inventors: Alissa Jourdan, Des Moines, IA (US); Jennifer Radosevich, Ankeny, IA (US)

Correspondence Address: DAVIS, BROWN, KOEHN, SHORS & ROBERTS, P.C.;THE FINANCIAL CENTER, 666 WALNUT STREET, SUITE 2500, DES MOINES, IA, 50309-3993, US

(21) Appl. No.: 10/884,548

(22) Filed: Jul. 1, 2004

Related U.S. Application Data
(60) Provisional application No. 60/484,763, filed on Jul. 3, 2003.

(51) Int. Cl. C12P 21/06; C12N 9/16; C12N 1/21; C07H 21/04; C12N 15/74

(52) U.S. Cl. 435/69.1; 435/196; 435/252.33; 435/488; 536/23.2

(57) ABSTRACT

The invention relates to a novel phytase excreted by an isolated fungal strain, nucleic acids encoding the phytase, prokaryotic and eukaryotic host cells transformed by the nucleic acids, and methods for producing the phytase.

This application claims priority to U.S. Provisional Patent Application Ser. No. 60/484,763, filed Jul. 3, 2003.

### BACKGROUND OF THE INVENTION

The invention relates generally to a novel phytase and gene and, more specifically, to a novel phytase enzyme that is added to animal feeds to reduce the need for phosphorus supplements in the animal diet and reduce the excretion of phosphate by the animal.

Phytic acid, or inositol hexaphosphate, is the primary storage form of phosphate in plant seeds. Monogastric animals, such as poultry or pigs, consume large amounts of plant material that contain high levels of phytic acid. However, these animals lack the necessary enzyme to degrade phytic acid and therefore are unable to utilize phytin phosphorus. Furthermore, it has been shown that the presence of phytic acid in feedstuffs acts as an antinutritive component in the diet (1, 8, 12). The lack of the enzyme able to hydrolyze phosphate from phytic acid, or phytase, and consequently the lack of adequate available phosphorus, has lead to diets supplemented with inorganic phosphate to ensure proper growth of the animals. Consequently, the excess of phytate phosphorus in animal manure has lead to important environmental issues such as polluted ponds and streams (2).

Phytase is an enzyme that hydrolyzes inorganic phosphate from phytic acid. Phytase can be found in certain plant seeds; however, some microorganisms, such as fungi, yeast, and bacteria, also have been found to produce the enzyme (3). It has been shown that addition of phytase to animal diets from microbe sources helps reduce the excretion of phosphate, having environmental benefits as well as reducing diet cost by partly or completely eliminating phosphorus supplements from the animal diet (2). There is a need to produce a novel phytase that would be superior to current phytase products.

Phosphorus is an essential nutrient required by all organisms. This element plays a central role in skeletal formation and is involved in numerous metabolic pathways. Accordingly, all animal diets must contain adequate amounts of this element. The detrimental effects of phosphorus-deficient diets on animal performance are well documented and include reduced appetite, bone malformation, and lowered fertility. Phytate accounts for more than 80% of the phosphorus found in the seeds and grains that make up animal feedstuffs (22). In this form phosphorus is biologically unavailable to monogastric animals (chicken, pigs, etc.) because they lack the enzyme phytase to catalyze the release of phosphorus from phytate (22, 23). In order to compensate for the lack of phytase activity and thus the unavailable phosphorus, animal diets are supplemented with inorganic phosphorus. Feed is often over supplemented with inorganic phosphorus and much of it passes through the animal, along with the undigested phytate, to the manure and into the environment. In areas of intensive livestock production this generates enormous problems with phosphorous pollution. Manure is spread on fields and since there is little phosphorus uptake by plants and phosphorus does not migrate through the soil like other nutrients, the excess phosphorus runs off into surface waters. This causes the eutrophication of surface waters; the process by which a body of water becomes rich in dissolved nutrients, like phosphates, causing algae blooms that deplete the water of oxygen. Ultimately, this dramatic decrease in dissolved oxygen levels results in massive fish kills (23). Phytate also acts as an anti-nutritive in the animal (8, 24). This compound has strong chelating abilities and can complex metal ions and proteins thereby decreasing their absorption in the intestinal tract (8, 24, 12). Breaking down or eliminating phytate would alleviate both of these problems.

Phytase is an enzyme that releases inorganic phosphate from phytate. The addition of microbial phytase to animal feed is well established as an effective and practical way of improving phytate digestibility, increasing phytate-phosphorus utilization, and decreasing the need for inorganic phosphorus supplementation (8, 25, 26, 12). The impact of phytase usage is considerable including increased phosphorus and calcium digestibility, improved feed intake, reduced phosphorus in manure, and reduced environmental phosphorus pollution. If phytase were used in the feed of all of the monogastric animals reared in the U.S. over a period of one year it would release phosphorus with a value of 168 million U.S dollars and would prevent 8.23×10⁴ tons of phosphate from entering the environment (27).

Phytase supplementation to the diets of poultry and swine may be the best example of an enzyme used to eliminate anti-nutritional compounds present in feed, giving appreciable benefits to animal nutrition and decreasing the phosphorus content in animal waste (8, 12, 22, 23, 24, 25). Phytase also has significant global implications in animal nutrition and environmental protection.

### SUMMARY OF THE INVENTION

Phytases for addition to animal feeds will advantageously have good thermostability to permit them to be added to the feed prior to pelleting yet retain satisfactory activity after being subjected to the rather harsh temperatures and conditions of pelleting. They will also advantageously have activity over a pH range which will again allow for retention of activity following processing of the animal feed and also exhibit activity in the digestive tract of the animal that ingests the feed. Further, the phytases will advantageously have physical characteristics which will allow them to stay uniformly distributed throughout the feed during processing and be readily dissolved into solution upon ingestion so as to be available to hydrolyze inorganic phosphate from phytic acid.

A search of a collection of soil samples revealed a fungal strain, identified as KPF0019, that secreted a phytase that was thermostable at 90° C., the temperature at which most manufacturers pellet feed. The thermostability of KPF0019 phytase is exhibited without requiring that it be coated. Coated phytase granules present a problem when attempting to mix them with feed carriers or blend them with other enzymes. The larger granules do not homogeneously mix with other traditional powdery mixes and the granules segregate during bagging. A phytase that is thermostable without coating a dry granule can be further developed as a thermostable phytase suitable for withstanding pelleting while providing it in a form for easy mixing.

The temperature activity profile of the KPF0019 phytase shows that the enzyme exerts more than half its activity at 37° C. when compared to the activity at the maximum in its profile. The phytase has a temperature optimum of approximately 55° C. and retains at least 30% of its maximum activity even after being heated to 90° C.-100° C. The phytase further has a pH optimum of about 5.5. Culture broth from the KPF0019 strain efficiently hydrolyzes phytic acid to intermediate reaction components (IP5, IP4, and IP3), as did a purified protein extracted from the broth. Additionally, the phytase from strain KPF0019 is not significantly more inhibited by the reaction product phosphate than the commercially available phytase sold under the name Natuphos (BASF). Sequence analysis of peptides from tryptic digest of KPF0019 phytase reveals the phytase is similar to a putative phytase sequence identified by BLAST search of the Neurospora crassa genome. Although there has been one report of phytase activity from Neurospora (7), there are no reports of the cloning of a phytase gene from Neurospora or evidence that the putative phytase gene identified in the Neurospora genome is not a pseudogene.

Following screening and biochemical characterization, a novel phytase gene was cloned from KPF0019. Reliable protein sequence data on the KPF0019 phytase was obtained by isolating the KPF0019 phytase using isoelectric focusing and subjecting it to tryptic digestion. The resulting peptides were separated and sequenced using a MALDI-TOF MS. Based on this information, oligonucleotides primers were designed and PCR was used to amplify the KPF0019 phytase gene sequence from KPF0019 genomic DNA. The amplification of the KPF0019 phytase gene, its nucleotide and deduced amino acid sequences are described.

The isolated nucleic acid sequence was used to transform host cells of Escherichia sp., Trichoderma sp. and Pichia sp. that were then grown under suitable conditions and expressed then phytase which was collected. Accordingly, both prokaryotic and eukaryotic host cells were transformed. An artificial nucleic acid was synthesized by converting each amino acid of the phytase-encoding nucleic acid sequence into the corresponding codon preferentially used by a selected host cell to be transformed using the artificial nucleic acid sequence. Specifically, a sequence codon-optimized for P. pastoris was synthesized, used to transform a host cell of P. pastoris which expressed the phytase.

The invention includes nucleic acid sequences that are at least 90% identical to SEQ ID NO. 1, preferably at least 85% identical to SEQ ID NO. 1, more preferably at least 75% identical to SEQ ID NO. 1, and more preferably at least 70% identical to SEQ ID NO. 1.

### BRIEF DESCRIPTION OF THE FIGURES

FIG. 1 is a graphical representation of the stabilites over time at 65° C. of two phytase enzymes, the KPF0019 phytase of the present invention and the phytase excreted by Aspergillus niger NRRL 3135.

FIG. 2 is a graphical representation of the stabilites over time at 90° C. of two phytase enzymes, the KPF0019 phytase of the present invention and the phytase excreted by A. niger NRRL 3135.

FIG. 3 is a graphical representation of the stabilites over time at 100° C. of two phytase enzymes, the KPF0019 phytase of the present invention and the phytase excreted by A. niger NRRL 3135.

FIG. 4 is a graphical representation of the temperature profile of the phytase from KPF0019 compared to the temperature profile of the phytase sold under the name Natuphos.

FIG. 5 is a graphical representation of the quantities of all hydrolysis products of phytic acid (IP6) observed after one hour of reaction with the culture broths of KPF0019 and A. niger NRRL3135.

FIG. 6 is a graphical representation of the quantities of all hydrolysis products of phytic acid (IP6) observed after four hours of reaction with the culture broths of KPF0019 and A. niger NRRL 3135.

FIG. 7 is a chart of the tryptic peptide sequences obtained from KPF0019 mapped onto a putative Neurospora crassa protein sequence.

FIG. 8 is a graphical representation of the pH activity profile of KPF0019 culture broth phytase.

FIG. 9 is a graphical representation of the temperature activity profile of KPF0019 culture broth phytase.

FIG. 10 is a graphical representation of the temperature stability profile of KPF0019 culture broth phytase.

FIG. 11 is a graphical representation of the pH stability of KPF0019 culture broth phytase.

FIG. 12 is a graphical representation of the phytase activities in total cell sonicate and sonicate supernatants after IPTG induction; activity was measured from IPTG induced BL21(DE3) cells carrying plasmids pEcPh-23 (middle columns of each set), pEcPh-28 (last columns of each set), and pET25-b(+) (first columns of each set).

FIG. 13 is a gel SDS-PAGE analyses of total cell sonicate and sonicate supernatant. The protein molecular weight marker is shown at the left. Samples are from IPTG induced cultures of BL21(DE3) cells carrying plasmids pEcPh-23, pEcPh-28, and pET25-b(+). The arrows represent the approximate size of the recombinant KPF0019 phytase protein.

FIG. 14 are schematics of the plasmids pTrPh-23 and pTrPh-28. CBHI_{SS}, T. reesei RUT-C30 cellobiohydrolase I signal sequence; P_{CBHI}, T. reesei RUT-C30 cellobiohydrolase I promoter; TT_{CBHI}, T. reesei RUT-C30 cellobiohydrolase I terminator; P_{ACT}, T. reesei RUT-C30 actin promoter; hph, E. coli hygromycin B resistance gene, bla, E. coli ampicillin resistance gene.

FIG. 15 is a graphical representation of the pH activity profile of rPhy produced by TrPh150 normalized to maximum activity at ph 5.5.

FIG. 16 is a graphical representation of the pH stability profile of rPhy produced by TrPh150, normalized to a zero time point at pH 5.5.

FIG. 17 is a graphical representation of the temperature activity profile of rPhy produced by TrPh150 normalized to maximum activity at 55° C.

FIG. 18 is a graphical representation of the temperature stability profile of rPhy produced by TrPh150 normalized to maximum activity at 50° C.

FIG. 19 is a schematic of plasmid pPpPh-23.

FIG. 20 is the DNA sequence of MFα-KPF-phy fusion junction and KEX2 cleavage site of plasmid pPpPh-23.

FIG. 21 is a graphical representation of the pH profile of rPhy produced by strain PpPh23-G1; normalized to maximum activity at pH 5.5.

FIG. 22 is a graphical representation of the pH stability of rPhy produced by strain PpPh23-G1; normalized to zero time point at pH 5.5

FIG. 23 is a graphical representation of the temperature profile of rPhy produced by strain PpPh23-G1; normalized to maximum activity at 60° C.

FIG. 24 is a graphical representation of the temperature stability rPhy produced by strain PpPh23-G1; normalized to maximum activity at 30° C.

FIG. 25 is an SDS-PAGE analysis of spent culture broth supernatant from P. pastoris transformant PpPh23-G1. Lanes 1-4: 20, 15, 10, and 5 μl, respectively, supernatant PpPh23-G1; lane 5, 15 μl KPF0019 purified phytase; lane 6, 20 μl of PpPh23-G1 supernatant from 50 mL shake-flask culture; lane 7, 20 μl of G-pKB (negative control) supernatant from 50 mL shake-flask culture; lane 8, protein MW standard. Data for K23-21 are not shown, but were similar to PpPh23-G1. Results are representative of two experiments.

FIG. 26A is an SDS-PAGE gel showing the Glycostaining of PpPh23-G1 spent culture broth supernatant; and FIG. 26B the same gel stained with GelCode Blue; results are representative of two experiments.

FIG. 27 is an SDS-PAGE gel showing in lane 1-2, 5 μl of Endo H treated and untreated PpPh23-G1 spent culture broth supernatant containing rPhy, respectively; lane 3-4, 5 μl treated and untreated G-pKB spent culture broth supernatant, respectively (negative controls); lane 5, protein MW standard. The lowest arrow represents Endo H protein, the top arrow represents glycosylated rPhy, and the middle arrow represents treated rPhy. Results are representative of three experiments

FIG. 28 is an SDS-PAGE gel of the expression of rPhy under fermentative conditions. Lane 1: 24 hr fermentation sample (15.6 μl); lane 2: 47 hr fermentation sample (15.6 μl); lane 3: 70 hr fermentation sample (5.0 μl); lane 4: 93 hr fermentation sample (5.0 μl); lane 5: culture-tube sample of rPhy produced from PpPh23-G1 (15.6 μl); lane 6: 93 hr fermentation sample (1.0 μl); lane 7: protein MW standard.

FIG. 29 is a graphical representation of the comparison of codon bias between the native KPF-phy gene and P. pastoris codon preferences. Data were generated using the on-line computer program Graphical Codon Usage Calculator. Dark bars represent codon preferences of P. pastoris and lighter bars represent codon usage in the native KPF-phy gene.

FIG. 30 is a schematic diagram of the plasmid pPpPh-21co.

FIG. 31 is an SDS-PAGE analysis of spent culture broth supernatant from P. pastoris transformants. Each lane represents 5 μl of culture broth supernatant collected after 24 h growth at 30° C. in 1 mL YPD broth. Lanes are labeled according to transformant number. The plus sign denotes supernatant from the positive control PpPh23-G1 and the dash denotes supernatant from the negative control G-pKB.

FIG. 32 is an SDS-PAGE analysis of the expression of rPhy^{CO} under fermentative conditions.

### DETAILED DESCRIPTION OF PREFERRED EMBODIMENTS

Definitions

As used in this specification, the term “phytase” refers to a protein or polypeptide that is capable of catalyzing the hydrolysis of phytic acid to release inorganic phosphate.

The specific activity of a phytase is defined as the number of units (U)/mg protein of a solution containing the phytase, wherein the phytase is detectable as a single band by SDS-PAGE. One unit is the amount of enzyme required to liberate one μmol of Pi per minute when the enzyme is incubated in a solution containing 50 mM acetate, pH 5.5, and 1.5 mM sodium phytate at 37° C.

Relative activity of phytase is defined throughout the specification as the activity of the phytase at a given temperature and/or pH compared to the activity of the phytase at the optimal temperature and/or pH of said phytase.

Prokaryotic host cells include cells from organisms including but are not limited to E. coli, Bacillus sp., Lactobacillus sp., and Lactococcus sp.

Eukaryotic host cells include cells from organisms including but are not limited to Aspergillus sp., Pichia sp., Saccharomyces sp., Trichoderma sp., and plants including but not limited to canola, corn and soya.

Hybridization can be performed under a variety of conditions ranging from high to low stringency. Stringency is sequence dependent and a truly accurate measurement of stringency can only be determined empirically. However, relative levels of stringency can be defined based on temperature and concentration of Na+ ions in the solutions used during hybridization and washing. In general, high stringency conditions are defined as salt concentrations between 0.01 to 1.5 M Na ion at pH 7.0 to 8.3 and temperatures of 30 C for short probes (10-50 nucleotides) and at least 60 C for long probes (greater than 50 nucleotides) (4, 10). Stringency can also be modulated through the addition of destabilizing agents such as formamide. Typical high stringency conditions would include hybridization in 50% formamide, 1 M NaCl, 1% SDS at 37 C and a wash in 0.1×SSC (20×SSC=3.0 M NaCl/0.3 M trisodium citrate) at 60 to 65 C (4, 10). Under these conditions a probe will only hybridize to a highly homologous (95-100%) target sequence.

Alternatively, hybridization can be done under low stringency conditions, which will allow mismatching of nucleotides to occur between the probe and target sequences. Typical low stringency conditions include hybridization in a solution of 30-35% formamide, 1 M NaCl, 1% SDS at 37 C and a wash in 1× or 2×SSC at 50-55 C (4, 10). Under these conditions the probe will hybridize to target sequences with which it is approximately 60-80% homologous.

A sequence comparison algorithm includes publicly available computer software which compares genetic sequences, such as the Vector NTI Suite 7,1 program sold by Invitrogen Corporation, Carlsbad, Calif.

### EXAMPLE 1—INITIAL SCREENING AND BIOCHEMICAL CHARACTERIZATION

A. Experimental Procedures

Materials. Phytic acid from rice and Fiske and Subbarow reducer were purchased from Sigma. All other chemicals and buffers were of the highest quality available from Fisher.

Strains and strain maintenance. Strains were selected from soil samples plated on phytic acid containing media. The plates were evaluated for clearing zones indicating phytate hydrolysis, as previously described by Shieh and Ware (15). Strains secreting putative phytases were grown on rich media for 7-14 days, and the culture broths assayed for phytase activity. Strains which appeared to secrete phytase activity were selected for isolation and placed on ISP2 slants (over 65 strains). From those slants, each strain was grown on ISP2 plates at 30° C. for 4-14 days (pass 1). The second passage onto ISP2 slants was also grown at 30° C. for 7-14 days to allow for sporulation, and then the strains were harvested with NTG and frozen at −80° C. for long term storage. With the exception of KPF0019 and KPF0174, all isolated strains were maintained in this manner and were viable after storage for up to 6 months. Since KPF0019 and KPF0174 were not viable after freezer storage of 2-3 months, those strains were maintained on ISP2 plates at 4° C. Expression of secreted phytases was obtained by growing the strains in K3 media (1.0 g/L peptonized milk, 1.0 g/L tryptone, and 5.0 g/L glucose) or K5 media (8.0 g/L Bacto Nutrient Broth and 1% glycerol) for 7 days with shaking at 200 rpm at 29° C. Broths were obtained by centrifuging the cultures for 10 minutes at 2000×g.

Phytase assays. In this assay, phosphate from the hydrolysis of phytate reacts with ammonium molybdate forming a phosphomolybdate complex. The amount of liberated phosphate is determined spectrophotometrically based on the formation of “molybedum blue” after reduction of the phosphomolybdate complex.

The following reagents are prepared. A 0.1 M acetate buffer, pH 5.5, is prepared by dissolving 8.2 g sodium acetate in 800 mL deionized water and the pH adjusted to 5.5 with glacial acetic acid. The solution is diluted to 1000 mL with deionized water. A 10 mM solution of phytic acid is prepared. The formula weight of each lot of phytic acid will vary since the weight of loss on drying (due to water) will vary on each bottle. The following is an example. Phytic acid lot 50K1123 from Sigma states on the bottle that the F.W. is 660.0, that it contains 6 mol/mol sodium, and the loss on drying was 3.3%; the F.W. of 660 g/mol assumes no sodium and no water (i.e. loss on drying); the mass of 6 mol of sodium (atomic weight 23 g/mol) is 138 g/mol, and this is added to the 660 g/mol to give 798 g/mol; the loss on drying is 3.3%, or 26.3 g/mol, and adding this gives a final mass of this phytic acid lot of 824.3 g/mol; in this case, dissolve 0.4116 g in 50 mL 0.1 M acetate buffer, pH 5.5. A 100% solution of trichloroacetic acid is prepared by pouring 150 mL deionized water into a 500 g bottle of trichloroacetic acid and shaken or stirred until the TCA dissolves. The solution is diluted to 100 mL with deionized water. A 5 N solution of H₂SO₄ is prepared by adding 139 mL concentrated sulfuric acid to 861 mL deionized water and stirring. Acid molybdate (2.5% in 5 N H₂SO₄) is prepared by dissolving 1.25 g ammonium molybdate in 50 mL 5 N H₂SO₄. A solution of 10% CaCl₂ is prepared by dissolving 10 g CaCl₂ in 70 mL deionized water and then diluted to 100 mL with deionized water. A granular phytase extraction buffer is prepared by combining 80 mL 0.1 M acetate buffer, ph 5.5, and 20 mL 10% CaCl₂. A solution of 1.5% CaCl₂/2.5% TCA is prepared by combining 37.5 mL 10% CaCl₂, 6.25 mL 100% TCA and 206 mL deionized water. A 100 mM potassium phosphate is prepared by dissolving 1.74 g potassium phosphate in 90 mL deionized water and diluting to 100 mL. A 4 mM phosphate standard is prepared by combining 4 mL 100 mM potassium phosphate with 96 mL 1.5% CaCl₂/2.5% TCA. A Fiske and Subbarow reducer is prepared by adding 1 g Fiske and Subbarow reducer to 6.3 mL deionized water; this solution is diluted 1:10 to prepare a working solution (combine 1 mL with 9 mL deionized water).

By way of example, a sample of phytase is prepared by weighing 0.1 g of the phytase sample, which is added to a 50 mL beaker together with 10 mL of the granular extraction buffer. The solution is stirred for 10 minutes and then centrifuged at 1600 g for 5 minutes. The supernatant is kept as a first dilution (1:100). Subsequent dilutions can be made with deionized water.

For the procedure, place 50 μL diluted enzyme solution into each well, or 50 μL water for blank. Pre-incubate at 37° C. for 5 minutes. In a separate container, also pre-incubate the 10 mM phytic acid at 37° C. for 5 minutes. Begin the reaction with the addition of 50 μL 10 mM phytic acid solution to the sample tubes and vortex. Incubate plate at 37° C. for 30 minutes and stop the reaction by adding 100 μL of 10% TCA to all wells.

Phosphate standards are prepared by combining the following volumes of 4 mM phosphate and 1.5% CaCl₂/2.5% TCA.

TABLE 1
CaCl₂/TCA for the indicated amounts of phosphate standard

| μmol phosphate | mL 4 mM phosphate | mL 1.5% CaCl₂/2.5% TCA |
|---|---|---|
| 0.0 | 0.00 | 1.00 |
| 0.2 | 0.05 | 0.95 |
| 0.4 | 0.10 | 0.90 |
| 0.6 | 0.15 | 0.85 |
| 0.8 | 0.20 | 0.80 |
| 1.0 | 0.25 | 0.75 |
| 1.2 | 0.30 | 0.70 |
| 1.4 | 0.35 | 0.65 |
| 1.6 | 0.40 | 0.60 |
| 1.8 | 0.45 | 0.55 |
| 2.0 | 0.50 | 0.50 |

For the color reaction, combine 20 μL phytase reaction or phosphate standard, 140 μL deionized water, 40 μL 2.5% acid molybdate, and 40 μL Fiske and Subbarow working solution (1:10) for a total of 240 μL. Let sit at room temperature 20 minutes, then measure A₇₅₀ spectophotometrically in a DU® 640 Beckman Spectrophotometer

Assays were performed with the above-described phytase assay method, with the exception that 100 μL of reaction instead of 20 μL was used for the color reaction. In some cases, culture broths were diluted to ensure the linearity of the phytase reaction. For temperature stability determination, samples were incubated for 5, 10, 20 and 60 minutes at 4° C. (control), 65° C., 90° C., and 100° C., then placed on ice and assayed for phytase activity at 37° C. For the temperature activity profile experiments, phytase assays were performed at 30° C., 37° C., 45° C., 50° C., 55° C., and 60° C. during the reaction. To determine if phosphate, one of the products of the phytase reaction, would inhibit the phytase activity of the strains, 0, 1.25, 2.5, 5.0, and 10.0 mM final phosphate was added to a standard reaction at 37° C.

HPLC analysis of phytase products. To obtain an analysis of the inositol phosphate products after the phytase reaction, the method according to Sandberg and Ahderinne (5) was followed. Briefly, inositol phosphate products were analyzed using a reverse-phase Supelcosil 25 cm×4.6 mm LC-18 column (Supelco) connected to an Agilent 1100 Series system and detected by differential refractometry. The isocratic mobile phase consisted of 0.05 M formic acid:methanol 49:51 and 1.5 mL/100 mL TBA-OH (tetrabutylammonium hydroxide), with the pH adjusted to 4.3 by addition of 9 M sulfuric acid. Under these conditions, phytic acid (IP6), inositol pentaphosphate (IP5), inositol tetraphosphate (IP4), and inositol triphosphate (IP3) could be separated. The IP's eluted with the same retention times as Sandberg and Ahderinne.

Phytase protein sequence analysis. Protein analysis was conducted. Secreted phytase was subjected to isoelectric focusing on IPG strips and stained for phytase activity in situ or with Coomassie. The relevant band was subjected to in-gel digestion with trypsin followed by MALDI-TOF MS analysis.

B. Results and Discussion

Secreted phytase activities. After a significant screening campaign, over 65 strains, ranging from bacteria, fungi, and actinomycetes, which secrete varying levels of phytase, were identified in terms of the increase in the absorbance at 750 nm, an indication of released phosphate from the substrate phytic acid in the reaction. The strains were grown in each respective medium, K3 and K5, for 7 days with shaking at 29° C. As previously observed in the broths of all of the strains (data not shown), there were major differences in the secreted phytase activities of strains. Generally, in the strains which secreted phytase activity, the measured activity in the broths were higher when the strains were grown in K5 media rather than K3 media. Additionally, two isolated strains, KPF0019 and KPF0174, secreted high phytase activity not only when compared to a non-secretor such as Trichoderma reesei RUT C30 but also when compared to a known phytase secretor, Aspergillus niger NRRL 3135. A. niger NRRL 3135 is the organism from which the phytase gene was cloned and subsequently overexpressed in another A. niger host for production of Natuphos (BASF). Because KPF0019 secreted high enough levels of phytase activity, the phytase from this isolated strain was chosen for further biochemical characterization to ascertain whether its properties were suitable for use as a commercial phytase product. KPF0019 has been deposited with the American Type Culture Collection, Manassas, Va., and is identified by accession number SD5361

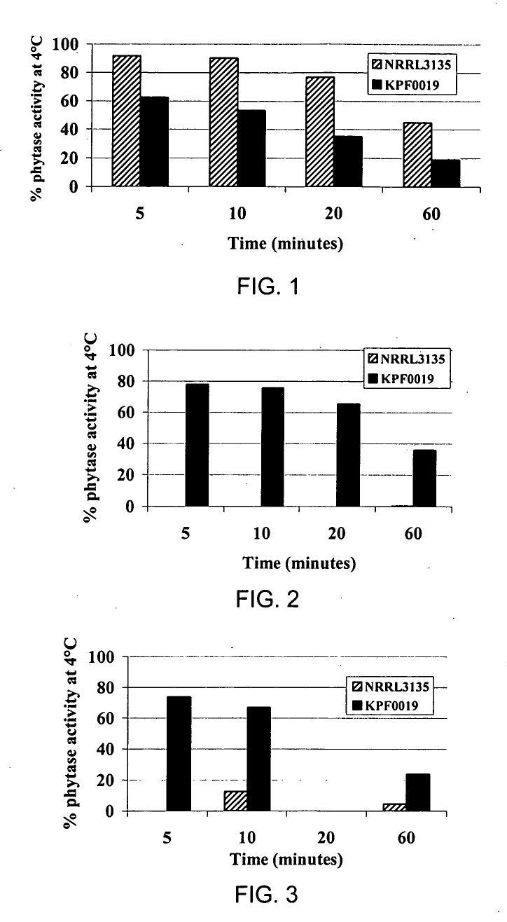

Secreted phytase thermostabilities. One of the most important desirable attributes of a commercial phytase is that of stability at high temperature. Thermostability determines how resistant a phytase will be to loss of activity during high pelleting temperatures in feed processing. To determine thermostability for the secreted phytase of strain KPF0019, culture broth was subjected to 65° C., 90° C. and 100° C. for various times, then assayed for phytase activity in a standard assay. For comparison, the broth from A. niger NRRL 3135 was included. As seen in FIG. 1, at 65° C., the phytase secreted from A. niger NRRL 3135 appears to be the more thermostable of the two broths tested. However, at 90° C. and 100° C. (FIGS. 2 and 3), the phytase from KPF0019 is considerably more stabile than A. niger NRRL 3135 phytase. Interestingly, the phytase secreted from KPF0019 is very thermostable at the typical pelleting temperature of 90° C. (FIG. 2).

Comparison to current phytase products. The thermostability of the phytase in the culture broth of the strain is compared with the thermostability of liquid commercial phytase preparations determined previously in Table 2. At 65° C., the stability of the phytase from KPF0019 is significantly better than the stability of the Natuphos and Finase phytase products. However, it is difficult to compare the stability of the phytase from KPF0019 at 100° C. for 20 minutes to the stability of Ronozyme (Table 3 and FIG. 3). It appears that there is no phytase activity left after this treatment; however, this appears to be an anomalous data point because at 10 minutes, 67% of the activity remains, while after 60 minutes, 24% of the activity remains (FIG. 3).

TABLE 2
Comparison of temperature stability of KPF0019 phytase with
current phytase products (% remaining activity)

| | % Stability at | % Stability at |
|---|---|---|
| Phytase sample | 65° C. for 20 min. | 100° C. for 20 min. |
| KPF0019 | 35 | (0) |
| Ronozyme Liquid | 77 | 43 |
| Natuphos Liquid | 18 | 33 |
| Finase Liquid | 2 | 14 |

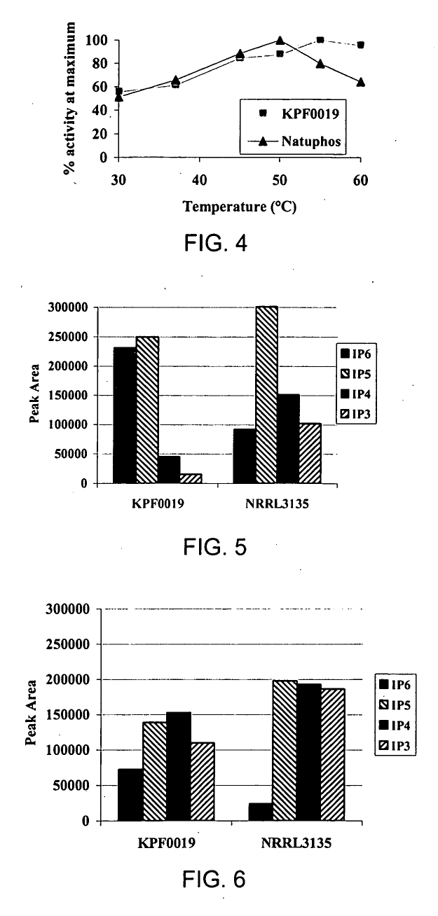

Since the phytase from KPF0019 was sufficiently thermostable, its temperature activity profile was determined. Temperature profile is important because certain enzymes may be stable and active at high temperatures, but not have sufficient activity at the relatively lower physiological activity of 37° C. FIG. 4 shows the temperature profile of the phytase from KPF0019. For comparison, the temperature profile of Natuphos is provided on the same figure. KPF0019 phytase exerts more than half its activity at 37° C. when compared to the activity at the 55° C. maximum in its temperature profile. This suggests that the activity of KPF0019 phytase would be sufficient under physiological conditions.

Because the temperature stability and profile of the phytase from KPF0019 was suitable for use commercially, an investigation into the efficiency of phytic acid (inositol hexaphosphate, or IP6) hydrolysis for the phytase was undertaken. Phytase from this strain was incubated with 5 mM phytic acid for 1 and 4 hours at 37° C., then the resultant products were separated by an HPLC method that analyzes for inositol phosphate compounds. Table 3 shows that the phytases efficiently hydrolyzed phytic acid within 1 hour, with 90%-92% of the IP6 being hydrolyzed. After 4 hours, 95-99% of the IP6 was hydrolyzed.

TABLE 3
Hydrolysis of phytic acid by secreted phytases

% hydrolysis
of phytic
acid (IP6) after:

| | 1 hour | 4 hours |
|---|---|---|
| KPF0019 | 90.6 | 95.5 |
| NRRL3135 | 92.4 | 98.5 |

To further analyze the hydrolysis of phytic acid by the phytases secreted by KPF0019 and A. niger NRRL 3135, the peak areas of the IP6, IP5, IP4, and IP3 peaks were monitored after 1 and 4 hours. IP2, IP1, and inositol were not observed as the HPLC method used cannot distinguish between these components of the reaction (5). As seen in FIG. 5, IP5, IP4, IP3, all hydrolysis products of phytic acid (IP6), were observed after one hour of reaction with the culture broths of both of these strains. Further hydrolysis of IP6 and IP5 along with the appearance of more IP4 and IP3 is evident after four hours of reaction (FIG. 6). These data confirm the utility of the secreted enzyme in the broth of KPF0019 as a phytase.

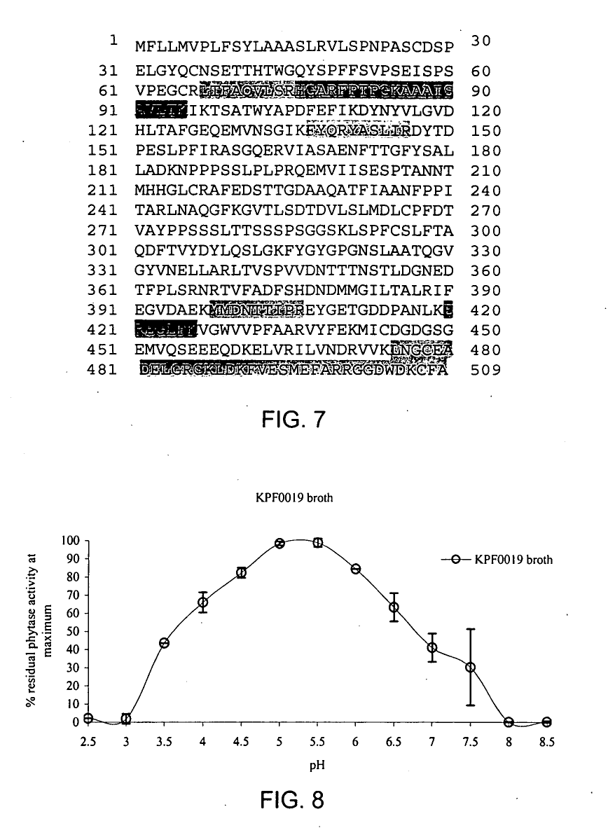

Since the secreted KPF0019 phytase exhibited desirable characteristics of a commercial phytase, the phytase was subjected to separation by isoelectric focusing and subsequent trypsin digestion. The resulting peptides were then separated and sequenced using a MALDI-TOF MS. A BLAST (Basic Local Alignment Search Tool) search of the Neurospora crassa genome with the peptide sequences obtained from KPF0019 phytase resulted in a partial match with a putative phytase from Neurospora crassa. The identified peptide fragments are mapped onto the putative Neurospora crassa protein sequence in FIG. 7. In the literature, there has been one report of phytase activity from Neurospora (7). However, there are no reports describing the cloning of a phytase gene from Neurospora or demonstrating that the putative phytase sequence identified in the Neurospora genome actually functions as a phytase. There is no evidence that the putative phytase gene identified in the Neurospora is not a pseudogene.

### EXAMPLE 2—CLONING OF THE NOVEL PHYTASE GENE

A. Experimental Procedures

Strains and Media. Fungal strain KPF0019 was grown in K5 media (Nutrient Broth [Difco, Detroit, Mich.], 10% glycerol). Bacterial strains were grown in either Luria-Burtani (LB) broth (per liter: Bacto tryptone, 10 g; Bacto yeast extract, 5 g; NaCl, 10 g), on LB agar (LB broth plus 1.5% agar). For plasmid maintenance, ampicillin (75 μg/mL) was added to LB broth and LB agar when needed.

Fungal genomic DNA extraction. KPF0019 was inoculated into 25 mL K5 broth and grown for 4-7 days at room temperature with shaking at 160-180 rpm. Mycelia were harvested onto Miracloth (Calbiochem, San Diego, Calif.) by vacuum filtration through a Buchner funnel, transferred to 50 mL disposable Flacon tubes and placed at −80° C. until ready to use. Genomic DNA extraction was based on a method by Saghai-Maroof et al. (10). Mycelia were frozen with liquid nitrogen and ground to a fine powder with a mortar and pestle. Approximately 300 mg of ground mycelia were transferred to a 50 mL disposable conical tube and mixed with 10 mL CTAB buffer (0.1 M Tris-HCl, pH 7.5, 1% cetyltrimethyl ammonium bromide, 0.7 M NaCl, 10 mM EDTA, 1% β-mercaptoethanol, 0.3 mg/mL Proteinase K). The mixture was incubated at 65° C. for 1 h. The mixture was cooled to room temperature, an equal volume of a 24:1 mixture of chloroform:isoamyl alcohol was added and the tube inverted multiple times to mix. The sample was centrifuged at 3000 rpm for 10 min and the supernate was transferred to a new tube. An equal volume of isopropanol was added to the supernate and mixed gently by inverting. Precipitated genomic DNA was spooled onto a drawn Pasteur pipette and placed in a microcentrifuge tube containing 1 mL 70% ethanol. The DNA was collected at the bottom of the tube by centrifugation at 14,000 rpm for 5 min. The supernate was removed and the genomic DNA allowed to air dry. Genomic DNA was resuspended in 1 mL of sterile 10 mM Tris-HCl, pH 8.0 and stored at −20° C.

PCR amplification of the KPF0019 putative phytase. Oligonucleotide primers were designed based on the sequence of the Neurospora crassa strain OK74A genome (GenBank accession number AABX01000000, locus NCU06351.1), contig 3.367 (scaffold 27) and synthesized by Integrated DNA Technologies, Inc. (Iowa City, Iowa). The full-length putative phytase gene from KPF0019 was amplified using the upstream primer Neu5-long (5′-ATGTTCCTCTTGATGGTTCCCTTGTTTAGCTAC-3′) in combination with the downstream primer Neu3 (5′-CTAAGCAAAACACTTGTCCCAATC-3′) in a PCR reaction using KPF0019 genomic DNA as template. Each 100 μL PCR reaction mixture contained approximately 300 ng genomic DNA, 500 nM of each primer, 200 uM dNTPs, 1×PFU Turbo Buffer (Stratagene, La Jolla, Calif.) and 2.5 U PFU Turbo Polymerase (Stratagene). The thermocycling program included one cycle at 95° C. (5 min) and 15 cycles of 95° C. (30 s), 50° C. (1 min) and 72° C. (2 min) immediately followed by 72° C. (10 min). An additional 25 cycles of 95° C. (30 s), 60° C. (1 min) and 72° C. (2 min) was followed by 72° C. (10 min) and an indefinite hold at 4° C. Amplified PCR products were visualized by electrophoresis through a 1% agarose gel containing 0.2 μg/mL ethidium bromide (9, 11). Gel slices containing the expected sized bands were excised and the DNA eluted using the Qiagen Gel Extraction Kit (Qiagen, Valencia, Calif.). Eluted PCR products were then sent to the Iowa State University DNA Sequencing and Synthesis Facility (Ames, Iowa), sequenced using the dideoxy method via the ABI PRISM Dye Terminator Cycle Sequencing Kit (Applied Biosystems, Foster City, Calif.) and analyzed with either the ABI Model 377 Prism DNA Sequencer or the ABI 3100 Genetic Analyzer (Applied Biosystems). The Neu5-long/Neu3 generated PCR product was ligated into plasmid pCR2.1-TOPO according to the manufacturer's suggestions (Invitrogen Life Technologies, Calsbad, Calif.). The ligation mix was transformed into Escherichia coli TOP10 electrocompetent cells, cells were plated on LB agar plus 75 μg/mL ampicillin and incubated overnight at 37° C. (9, 11). Transformants were transferred to 1.5 mL LB broth plus 75 μg/mL ampicillin and grow overnight at 37° C. with shaking. Plasmid was prepared from each transformant using the Qiagen Plasmid Miniprep Kit (Qiagen) and 10 μL of the plasmid preparation was digested with EcoRI to confirm the size of the insert. Two plasmids containing the correct sized inserts were sent to Iowa State University's DNA Sequencing and Synthesis Facility for sequencing (as described above). In silico analysis of the KPF0019-Phy gene sequence was performed with Vector NTI v.7 Sequence Analysis software (InforMax, Inc., Frederick, Md.). This software was used to do pairwise similarity alignments, generate restriction maps, deduce amino acid sequences and theoretically determine biochemical properties of proteins.

B. Results and Discussion

Amino acid sequence data for the KPF0019-PHY was obtained using isoelectric focusing to identify the protein responsible for phytase activity in strain KPF0019 and using MALDI-TOF MS to determine the amino acid sequence of several tryptic peptide fragments of the KPF0019-P. Based on this information we were able to determine that the KPF0019-PHY protein closely resembled a Neurospora crassa putative phosphatase protein (GenBank accession number AABX01000000, locus NCU06351.1). Based on this information and the N. crassa genomic sequence, a series of oligonucleotide primers were designed to PCR amplify various segments of the KPF0019-PHY gene from KPF0019 genomic DNA (gDNA). DNA sequencing was performed on all of the PCR products generated (data not shown).

One set of oligonucleotide primers (Neu5-long and Neu3) amplified the entire coding region of the KPF0019-PHY gene. The DNA sequence is set out in Table 4 wherein; the translational start and translation stop are underlined and the 66 bp native intron is highlighted.

TABLE 4
DNA Sequence (SEQ ID NO. 1) of the KPF0019 phytase gene-coding region

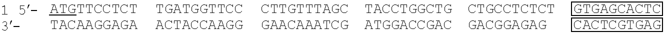

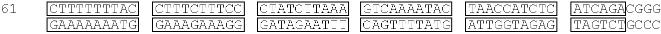

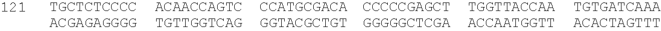

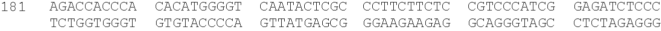

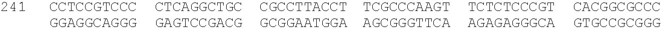

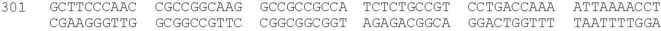

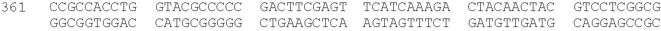

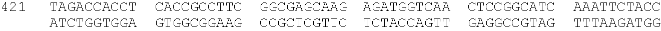

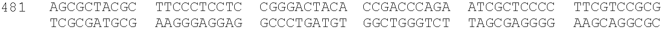

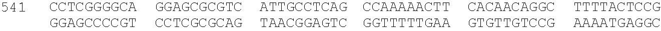

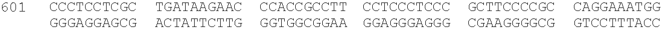

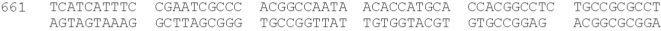

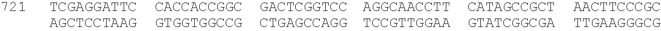

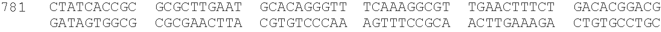

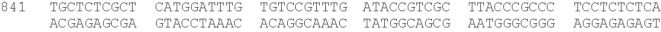

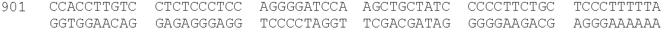

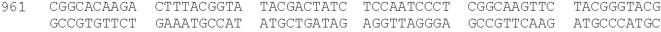

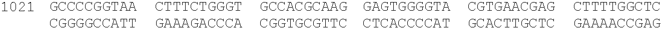

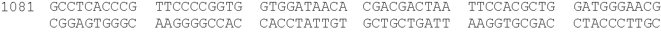

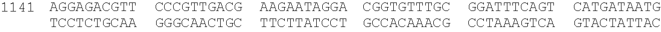

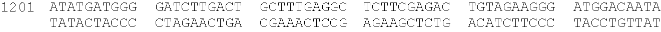

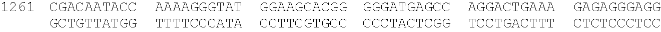

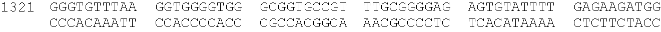

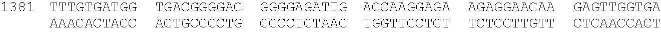

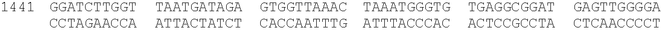

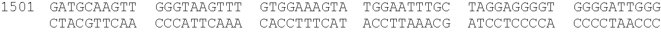

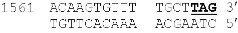

The DNA sequence of this PCR product matches the DNA sequences of several other PCR products that were amplified from KPF0019 gDNA using various oligonucleotide primers spanning the entire putative coding region (data not shown). This result suggested that the correct gene sequence from KPF0019 had been amplified. Alignment of the KPF0019-PHY gene sequence with the N. crassa genome (contig 3.367, scaffold 27, locus NCU06351.1) also provided further evidence that the correct DNA sequence from KPF0019 had been amplified. See Table 5, wherein nucleotide changes are shown in the KPF0019-Phy gene sequence and underlined in bold; deletions are shown as a box and insertions are shown as an asterisk; each sequence is numbered separately; the native introns of both sequences are shown highlighted. Additionally, The nucleotide sequence of the KPF0019-Phy gene (without the intron) and its deduced amino acid sequence are presented in Table. 5A. The aligned nucleotide and amino acid sequences were prepare using the AlignX program of the Vector NTI Suite 7.1 software program of Invitrogen Corp., Carlsbad, Calif., set to pairwise alignment, default settings, with a gap opeinin penalty=15, gap extension penalty=6.66, and gap separation range=8.

TABLE 5
Alignment of the KPF0019-Phy gene sequence with the N. crassa genome (contig 3.367, scaffold 27, locus NCU06351.1)

| | | | | | | |
|---|---|---|---|---|---|---|
| 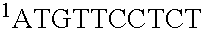 | TGATGGTTCC | CTTGTTTAGC | TACCTGGCTG | CTGCTTCTCT | 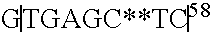 | N. crassa |
| 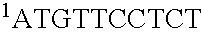 | TGATGGTTCC | CTTGTTTAGC | TACCTGGCTG | 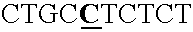 | 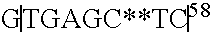 | KPF0019-PHY |
| 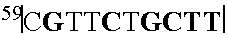 | 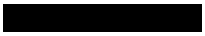 | 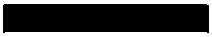 | 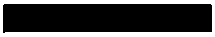 | 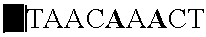 | CATCAGACGA¹¹⁸ | N. crassa |
| 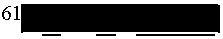 | 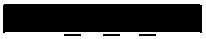 | 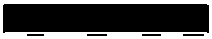 | 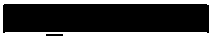 | 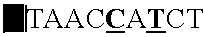 | CATCAGACGG¹¹⁹ | KPF0019-PHY |
| ¹¹⁹GTACTATCCC | CAAATCCAGC | ATCATGCGAC | AGCCCAGAGC | TTGGTTACCA | ATGTAACTCA¹⁷⁸ | N. crassa |
| ¹²⁰GTGCTCTCCC | CACAACCAGT | CCCATGCGAC | ACCCCCGAGC | TTGGTTACCA | ATGTGATCAA¹⁷⁹ | KPF0019-PHY |
| ¹⁷⁹GAGACAACCC | ACACATGGGG | TCAATACTCG | CCCTTCTTCT | CCGTCCCGTC | GGAAATCTCC²³⁸ | N. crassa |
| ¹⁸⁰AAGACCACCC | ACACATGGGG | TCAATACTCG | CCCTTCTTCT | CCGTCCCATC | GGAGATCTCC²³⁹ | KPF0019-PHY |
| ²³⁹CCCTCCGTTC | CCGAGGGTTG | CCGCCTCACC | TTTGCCCAAG | TTCTTTCTCG | CCACGGCGCT²⁹⁸ | N. crassa |
| ²⁴⁰CCCTCCGTCC | CCTCAGGCTG | CCGCCTTACC | TTCGCCCAAG | TTCTCTCCCG | TCACGGCGCC²⁹⁹ | KPF0019-PHY |
| ²⁹⁹CGCTTCCCAA | CTCCCGGAAA | AGCCGCCGCC | ATCTCCGCCG | TTCTCACCAA | GATCAAAACC³⁵⁸ | N. crassa |
| ³⁰⁰CGCTTCCCAA | CCGCCGGCAA | GGCCGCCGCC | ATCTCTGCCG | TCCTGACCAA | AATTAAAACC³⁵⁹ | KPF0019-PHY |
| ³⁵⁹TCCGCCACCT | GGTACGCCCC | CGACTTCGAG | TTCATCAAAG | ACTACAACTA | TGTCCTCGGC⁴¹⁸ | N. crassa |
| ³⁶⁰TCCGCCACCT | GGTACGCCCC | CGACTTCGAG | TTCATCAAAG | ACTACAACTA | CGTCCTCGGC⁴¹⁹ | KPF0019-PHY |
| ⁴¹⁹GTCGACCACC | TCACTGCCTT | TGGCGAGCAA | GAGATGGTCA | ACTCGGGCAT | CAAATTCTAC⁴⁷⁸ | N. crassa |
| ⁴²⁰GTAGACCACC | TCACCGCCTT | CGGCGAGCAA | GAGATGGTCA | ACTCCGGCAT | CAAATTCTAC⁴⁷⁹ | KPF0019-PHY |
| ⁴⁷⁹CAACGCTACG | CTTCCCTCAT | CCGGGACTAC | ACTGACCCAG | AATCCCTCCC | CTTTATCCGT⁵³⁸ | N. crassa |
| ⁴⁸⁰CAGCGCTACG | CTTCCCTCCT | CCGGGACTAC | ACCGACCCAG | AATCGCTCCC | CTTCGTCCGC⁵³⁹ | KPF0019-PHY |
| ⁵³⁹GCCTCGGGGC | AGGAGCGCGT | CATTGCCTCA | GCTGAGAACT | TCACCACTGG | GTTCTACTCT⁵⁹⁸ | N. crassa |
| ⁵⁴⁰GCCTCGGGGC | AGGAGCGCGT | CATTGCCTCA | GCCAAAAACT | TCACAACAGG | CTTTTACTCC⁵⁹⁹ | KPF0019-PHY |
| ⁵⁹⁹GCCCTCCTTG | CCGACAAGAA | CCCACCCCCT | TCCTCCCTCC | CGCTTCCCCG | CCAGGAGATG⁶⁵⁸ | N. crassa |
| ⁶⁰⁰GCCCTCCTCG | CTGATAAGAA | CCCACCGCCT | TCCTCCCTCC | CGCTTCCCCG | CCAGGAAATG⁶⁵⁹ | KPF0019-PHY |
| ⁶⁵⁹GTCATCATCT | CCGAATCGCC | CACGGCCAAC | AACACCATGC | ACCACGGTCT | CTGCCGCGCC⁷¹⁸ | N. crassa |
| ⁶⁶⁰GTCATCATTT | CCGAATCGCC | CACGGCCAAT | AACACCATGC | ACCACGGCCT | CTGCCGCGCC⁷¹⁹ | KPF0019-PHY |
| ⁷¹⁹TTTGAGGACT | CCACCACCGG | CGATGCGGCC | CAGGCGACCT | TTATAGCTGC | CAACTTCCCG⁷⁷⁸ | N. crassa |
| ⁷²⁰TTCGAGGATT | CCACCACCGG | CGACTCGGTC | CAGGCAACCT | TCATAGCCGC | TAACTTCCCG⁷⁷⁹ | KPF0019-PHY |
| ⁷⁷⁹CCCATCACCG | CGCGGTTGAA | TGCGCAGGGT | TTCAAAGGCG | TCACTCTTTC | CGACACGGAC⁸³⁸ | N. crassa |
| ⁷⁸⁰CCTATCACCG | CGCGCTTGAA | TGCACAGGGT | TTCAAAGGCG | TTGAACTTTC | TGACACGGAC⁸³⁹ | KPF0019-PHY |
| ⁸³⁹GTGCTCTCGC | TCATGGATCT | CTGCCCCTTT | GACACCGTCG | CTTACCCGCC | CTCCTCCTCT⁸⁹⁸ | N. crassa |
| ⁸⁴⁰GTGCTCTCGC | TCATGGATTT | GTGTCCGTTT | GATACCGTCG | CTTACCCGCC | CTCCT▪▪▪CT⁸⁹⁶ | KPF0019-PHY |
| ⁸⁹⁹CTCACCACCT | CGTCCTCTCC | CTCGGGGGGA | AGCAAGCTC* | **TCCCCCTT | TTGCTCCCTT⁹⁵⁵ | N. crassa |
| ⁸⁹⁷CTCACCACCT | TGTCCTCTCC | CTCCAGGGGA | TCCAAGCTGC | TATCCCCCTT | CTGCTCCCTT⁹⁵⁶ | KPF0019-PHY |
| ⁹⁵⁶TTTACCGCTC | AAGACTTTAC | CGTGTACGAC | TACCTCCAGT | CCCTCGGCAA | GTTCTACGGC¹⁰¹⁵ | N. crassa |
| ⁹⁵⁷TTTACGGCAC | AAGACTTTAC | GGTATACGAC | TATCTCCAAT | CCCTCGGCAA | GTTCTACGGG¹⁰¹⁶ | KPF0019-PHY |
| ¹⁰¹⁶TACGGCCCGG | GTAATTCTCT | GGCTGCCACG | CAGGGGGTGG | GGTACGTGAA | CGAGCTTTTG¹⁰⁷⁵ | N. crassa |
| ¹⁰¹⁷TACGGCCCCG | GTAACTTTCT | GGGTGCCACG | CAAGGAGTGG | GGTACGTGAA | CGAGCTTTTG¹⁰⁷⁶ | KPF0019-PHY |
| ¹⁰⁷⁶GCTCGCCTCA | CGGTTTCCCC | GGTGGTGGAT | AACACGACCA | CCAATTCCAC | GCTGGACGGG¹¹³⁵ | N. crassa |
| ¹⁰⁷⁷GCTCGCCTCA | CCCGTTCCCC | GGTGGTGGAT | AACACGACGA | CTAATTCCAC | GCTGGATGGG¹¹³⁶ | KPF0019-PHY |
| ¹¹³⁶AACGAGGACA | CGTTTCCGCT | GAGTAGGAAC | AGGACGGTGT | TTGCGGATTT | CAGTCATGAT¹¹⁹⁵ | N. crassa |
| ¹¹³⁷AACGAGGAGA | CGTTCCCGTT | GACGAAGAAT | AGGACGGTGT | TTGCGGATTT | CAGTCATGAT¹¹⁹⁶ | KPF0019-PHY |
| ¹¹⁹⁶AATGATATGA | TGGGGATCTT | GACTGCTTTG | AGAATCTTTG | AGGGGGTGGA | TGCGGAGAAG¹²⁵⁵ | N. crassa |
| ¹¹⁹⁷AATGATATGA | TGGGGATCTT | GACTGCTTTG | AGGCTCTTCG | AGACTGT▪▪▪ | ▪▪▪▪▪AGAAG¹²⁴⁷ | KPF0019-P |
| ¹²⁵⁶ATGATGGATA | ATACGACCAT | ACCGAGAGAG | TACGGGGAGA | CTGGCGATGA | TCCGGCAAAT¹³¹⁵ | N. crassa |
| ¹²⁴⁸G▪GATGGACA | ATACGACAAT | ACCAAAAGGG | TATGGAAGCA | CGGGGGATGA | GCCAGGA▪▪▪¹³⁰³ | KPF0019-PHY |
| ¹³¹⁶TTGAAAGAGA | GGGAGGGCTT | GTTCAAGGTT | GGTTGGGTGG | TGCCATTTGC | GGCGAGGGTG¹³⁷⁵ | N. crassa |
| ¹³⁰⁴CTGAAAGAGA | GGGAGGGGGT | GTTTAAGGTG | GGGTGGGCGG | TGCCGTTTGC | GGGGAGAGTG¹³⁶⁴ | KPF0019-PHY |
| ¹³⁷⁶TATTTTGAAA | AGATGATTTG | TGATGGGGAT | GGGAGTGGAG | AGATGGTTCA | GAGCGAGGAG¹⁴³⁵ | N. crassa |
| ¹³⁶⁵TATTTTGAGA | AGATGGTTTG | TGATGGTGAC | GGGGACGGGG | AGATTGACCA | AGGAGAAGAG¹⁴²⁴ | KPF0019-P |
| ¹⁴³⁶GAACAGGACA | AGGAGTTGGT | GAGGATCTTG | GTTAACGATA | GAGTGGTTAA | ACTAAATGGA¹⁴⁹⁵ | N. crassa |
| ¹⁴²⁵GAACAAGA▪▪ | ▪▪▪▪GTTGGT | GAGGATCTTG | GTTAATGATA | GAGTGGTTAA | ACTAAATGGG¹⁴⁷⁸ | KPF0019-PHY |
| ¹⁴⁹⁶TGTGAGGCCG | ATGAGTTGGG | GAGGTGTAAG | TTGGATAAAT | TTGTAGAGAG | TATGGAGTTT¹⁵⁵⁵ | N. crassa |
| ¹⁴⁷⁹TGTGAGGCGG | ATGAGTTGGG | GAGATGCAAG | TTGGGTAAGT | TTGTGGAAAG | TATGGAATTT¹⁵³⁸ | KPF0019-PHY |
| ¹⁵⁵⁶GCTAGGAGGG | GTGGAGATTG | GGACAAGTGT | TTTGCTTAG¹⁵⁹⁴ | | | N. crassa |
| ¹⁵³⁹GCTAGGAGGG | GTGGGGATTG | GGACAAGTGT | TTTGCTTAG¹⁵⁷⁷ | | | KPF0019-PHY |

TABLE 5A

The nucleotide sequence of the KPF0019-Phy gene
and its deduced amino acid sequence (SEQ ID NO. 2)

```
M F L L M V P L F S Y L
ATG TTC CTC TTG ATG GTT CCC TTG TTT AGC TAC CTG
TAC AAG GAG AAC TAC CAA GGG AAC AAA TCG ATG GAC
A A A S L R V L S P Q P
GCT GCT GCC TCT CTG CGG GTG CTC TCC CCA CAA CCA
CGA CGA CGG AGA GAC GCC CAC GAG AGG GGT GTT GGT
  V P C D T P E L G Y Q C
GTC CCA TGC GAC ACC CCC GAG CTT GGT TAC CAA TGT
CAG GGT ACG CTG TGG GGG CTC GAA CCA ATG GTT ACA
  D Q K T T H T W G Q Y S
GAT CAA AAG ACC ACC CAC ACA TGG GGT CAA TAC TCG
CTA GTT TTC TGG TGG GTG TGT ACC CCA GTT ATG AGC
  P F F S V P S E I S P S
CCC TTC TTC TCC GTC CCA TCG GAG ATC TCC CCC TCC
GGG AAG AAG AGG CAG GGT AGC CTC TAG AGG GGG AGG
  V P S G C R L T F A Q V
GTC CCC TCA GGC TGC CGC CTT ACC TTC GCC CAA GTT
CAG GGG AGT CCG ACG GCG GAA TGG AAG CGG GTT CAA
  L S R H G A R F P T A G
CTC TCC CGT CAC GGC GCC CGC TTC CCA ACC GCC GGC
GAG AGG GCA GTG CCG CGG GCG AAG GGT TGG CGG CCG
  K A A A I S A V L T K I
AAG GCC GCC GCC ATC TCT GCC GTC CTG ACC AAA ATT
TTC CGG CGG CGG TAG AGA CGG CAG GAC TGG TTT TAA
  K T S A T W Y A P D F E
AAA ACC TCC GCC ACC TGG TAC GCC CCC GAC TTC GAG
TTT TGG AGG CGG TGG ACC ATG CGG GGG CTG AAG CTC
F I K D Y N Y V L G V D
TTC ATC AAA GAC TAC AAC TAC GTC CTC GGC GTA GAC
AAG TAG TTT CTG ATG TTG ATG CAG GAG CCG CAT CTG
  H L T A F G E Q E M V N
CAC CTC ACC GCC TTC GGC GAG CAA GAG ATG GTC AAC
GTG GAG TGG CGG AAG CCG CTC GTT CTC TAC CAG TTG
  S G I K F Y Q R Y A S L
TCC GGC ATC AAA TTC TAC CAG CGC TAC GCT TCC CTC
AGG CCG TAG TTT AAG ATG GTC GCG ATG CGA AGG GAG
  L R D Y T D P E S L P F
CTC CGG GAC TAC ACC GAC CCA GAA TCG CTC CCC TTC
GAG GCC CTG ATG TGG CTG GGT CTT AGC GAG GGG AAG
  V R A S G Q E R V I A S
GTC CGC GCC TCG GGG CAG GAG CGC GTC ATT GCC TCA
CAG GCG CGG AGC CCC GTC CTC GCG CAG TAA CGG AGT
  A K N F T T G F Y S A L
GCC AAA AAC TTC ACA ACA GGC TTT TAC TCC GCC CTC
CGG TTT TTG AAG TGT TGT CCG AAA ATG AGG CGG GAG
  L A D K N P P P S S L P
CTC GCT GAT AAG AAC CCA CCG CCT TCC TCC CTC CCG
GAG CGA CTA TTC TTG GGT GGC GGA AGG AGG GAG CCC
  L P R Q E M V I I S E S
CTT CCC CGC CAG GAA ATG GTC ATC ATT TCC GAA TCG
GAA GGG CCC GTC CTT TAC CAG TAG TAA AGG CTT AGC
  P T A N N T M H H G L C
CCC ACG GCC AAT AAC ACC ATG CAC CAC GGC CTC TGC
GGG TGC CGG TTA TTG TGG TAC GTG GTG CCG GAG ACG
  R A F E D S T T G D S V
CGC CCC TTC GAG GAT TCC ACC ACC GGC GAC TCG GTC
GCG CGG AAG CTC CTA AGG TGG TGG CCG CTG AGC CAG
  Q A T F I A A N F P P I
CAG GCA ACC TTC ATA GCC GCT AAC TTC CCG CCT ATC
GTC CGT TGG AAG TAT CGG CGA TTG AAG GGC GGA TAG
  T A R L N A Q G F K G V
ACC GCG CGC TTG AAT GCA CAG GGT TTC AAA GGC GTT
TGG CGC GCG AAC TTA CGT GTC CCA AAG TTT CCG CAA
  E L S D T D V L S L M D
GAA CTT TCT GAC ACG GAC GTG CTC TCG CTC ATG GAT
CTT GAA AGA CTG TGC CTG CAC GAG AGC GAG TAC CTA
  L C P F D T V A Y P P S
TTG TGT CCG TTT GAT ACC GTC GCT TAC CCG CCC TCC
AAC ACA GGC AAA CTA TGG CAG CGA ATG GGC GGG AGG
  S L T T L S S P S R G S
TCT CTC ACC ACC TTG TCC TCT CCC TCC AGG GGA TCC
AGA GAG TGG TGG AAC AGG AGA GGG AGG TCC CCT AGG
  K L L S P F C S L F T A
AAG CTG CTA TCC CCC TTC TGC TCC CTT TTT ACG GCA
TTC GAC CAT AGG GGG AAG ACG ACG GAA AAA TGC CGT
  Q D F T V Y D Y L Q S L
CAA GAC TTT ACG GTA TAC GAC TAT CTC CAA TCC CTC
GTT CTG AAA TGC CAT ATG CTG ATA GAG GTT AGG GAG
  G K F Y G Y G P G N F L
GGC AAG TTC TAC GGG TAC GGC CCC GGT AAC TTT CTG
CCG TTC AAG ATG CCC ATG CCG GGG CCA TTG AAA GAC
  G A T Q G V G Y V N E L
GGT GCC ACG CAA GGA GTG GGG TAC GTG AAC GAG CTT
CCA CGG TGC GTT CCT CAC CCC ATG CAC TTG CTC GAA
  L A R L T R S P V V D N
TTG GCT CGC CTC ACC CGT TCC CCG GTG GTG GAT AAC
AAC CGA GCG GAG TGG GCA AGG GGC CAC CAC CTA TTG
  T T T N S T L D G N E E
ACG ACG ACT AAT TCC ACG CTG GAT GGG AAC GAG GAG
TGC TGC TGA TTA AGG TGC GAC CTA CCC TTG CTC CTC
  T F P L T K N R T V F A
ACG TTC CCG TTG ACG AAG AAT AGG ACG GTG TTT GCG
TGC AAG GGC AAC TGC TTC TTA TCC TGC CAC AAA CGC
  D F S H D N D M  M G I L
GAT TTC AGT CAT GAT AAT GAT ATG ATG GGG ATC TTG
CTA AAG TCA GTA CTA TTA CTA TAC TAC CCC TAG AAC
  T A L R L F E T V E G M
ACT GCT TTG AGG CTC TTC GAG ACT GTA GAA GGG ATG
TGA CGA AAC TCC GAG AAG CTC TGA CAT CTT CCC TAC
  D N T T I P K G Y G S T
GAC AAT ACG ACA ATA CCA AAA GGG TAT GGA AGC ACG
CTG TTA TGC TGT TAT GGT TTT CCC ATA CCT TCG TGC
  G D E P G L K E R E G V
GGG GAT GAG CCA GGA CTG AAA GAG AGG GAG GGG GTG
CCC CTA CTC GGT CCT GAC TTT CTC TCC CTC CCC CAC
  F K V G W A V P F A G R
TTT AAG GTG GGG TGG GCG GTG CCG TTT GCG GGG AGA
AAA TTC CAC CCC ACC CGC CAC GGC AAA CGC CCC TCT
  V Y F E K M V C D G D G
GTG TAT TTT GAG AAG ATG GTT TGT GAT GGT GAC GGG
CAC ATA AAA CTC TTC TAC CAA ACA CTA CCA CTG CCC
  D G E I D Q G E E E Q E
GAC GGG GAG ATT GAC CAA GGA GAA GAG GAA CAA GAG
CTG CCC CTC TAA CTG GTT CCT CTT CTC CTT GTT CTC
  L V R I L V N D R V V K
TTG GTG AGG ATC TTG GTT AAT GAT AGA GTG GTT AAA
AAC CAC TCC TAG AAC CAA TTA CTA TCT CAC CAA TTT
  L N G C E A D E L G R C
CTA AAT GGG TGT GAG GCG GAT GAG TTG GGG AGA TGC
GAT TTA CCC ACA CTC CGC CTA CTC AAC CCC TCT ACG
  K L G K F V E S M E F A
AAG TTG GGT AAG TTT GTG GAA AGT ATG GAA TTT GCT
TTC AAC CCA TTC AAA CAC CTT TCA TAC CTT AAA CGA
  R R G G D W D K C F A *
AGG AGG GGT GGG GAT TGG GAC AAG TGT TTT GCT TAG
TCC TCC CCA CCC CTA ACC CTG TTC ACA AAA CGA ATC
```

As set out in Table 5, the DNA sequence of the KPF0019-PHY gene is 85.8% identical to the DNA sequence of the N. crassa putative phosphatase gene. Nucleotide acid changes in the KPF0019-Phy gene are shown underlined in bold, codon deletions in the KPF0019-Phy gene sequence are shown as boxes and the insertions are shown as asterisks.

TABLE 6

Alignment of the deduced amino acid sequences of the KPF0019
phytase protein (SEQ ID NO. 2) with the N. crassa putative
phosphatase protein

| | | | | | |
|---|---|---|---|---|---|
| KPF0019-P | ¹MFLLMVPLFS | YLAAASLRVL | SPQPVPCDTP | ELGYQCDQKT | THTWGQYSPF⁴⁹ |
| N. crassa | ¹MFLLMVPLFS | YLAAASLRVL | SPNPASCDSP | ELGYQCN SET | THTWGQYSPF⁴⁹ |
| KPF0019-P | FSVPSEISPS | VPSGCRLTFA | QVLSRHGARF | PTAGKAAAIS | AVLTKIKTSA⁹⁹ |
| N. crassa | FSVPSEISPS | VPEGCRLTFA | QVLSRHGARF | PTPGKAAAIS | AVLTKIKTSA⁹⁹ |
| KPF0019-P | TWYAPDFEFI | KDYNYVLGVD | HLTAFGEQEM | VNSGIKFYQR | YASLLRDYTD¹⁴⁹ |
| N. crassa | TWYAPDFEFI | KDYNYVLGVD | HLTAFGEQEM | VNSGIKFYQR | YASLIRDYTD¹⁴⁹ |
| KPF0019-P | PESLPFVRAS | GQERVIASAK | NFTTGFYSAL | LADKNPPPSS | LPLPRQEMVI¹⁹⁹ |
| N. crassa | PESLPF IRAS | GQERVIASAE | NFTTGFYSAL | LADKNPPPSS | LPLPRQEMVI¹⁹⁹ |
| KPF0019-P | ISESPTANNT | MHHGLCRAFE | DSTTGDSVQA | TFIAANFPPIT | ARLNAQGFK²⁴⁹ |
| N. crassa | ISESPTANNT | MHHGLCRAFE | DSTTGDAAQA | TFIAANFPPIT | ARLNAQGFK²⁴⁹ |
| KPF0019-P | GVELSDTDVL | SLMDLCPFDT | VAYPPSS▪LT | TLSSPSRGSK | LLSPFCSLFT²⁹⁸ |
| N. crassa | GVTLSDTDVL | SLMDLCPFDT | VAYPPSSSLT | TSSSPSGGS* | KLSPFCSLFT²⁹⁸ |
| KPF0019-P | AQDFTVYDYL | QSLGKFYGYG | PGNFLGATQG | VGYVNELLAR | LTRSPVVDNT³⁴⁸ |
| N. crassa | AQDFTVYDYL | QSLGKFYGYG | PGNSLAATQG | VGYVNELLAR | LTVSPVVDNT³⁴⁸ |
| KPF0019-P | TTNSTLDGNE | ETFPLTKNRT | VFADFSHDND | MMGILTALRL | FETVEG▪▪▪M³⁹⁵ |
| N. crassa | TTNSTLDGNE | DTFPLSRNRT | VFADFSHDND | MMGILTALRI | FEGVDAEKMM³⁹⁸ |
| KPF0019-P | DNTTIPKGYG | STGDEPG▪LK | EREGVFKVGW | AVPFAGRVYF | EKMVCDGDGD⁴⁴⁵ |
| N. crassa | DNTTIPREYG | ETGDDPANLK | EREGLFKVGW | VVPFAARVYF | EKMICDGDGS⁴⁴⁸ |
| KPF0019-P | GEIDQGEEE▪ | ▪QELVRILVN | DRVVKLNGCE | ADELGRCKLG | KFVESMEFAR⁴⁹³ |
| N. crassa | GEMVQSEEEQ | DKELVRILVN | DRVVKLNGCE | ADELGRCKLD | KFVESMEFAR⁴⁹⁹ |
| KPF0019-P | RGGDWDKCFA⁵⁰³ | | | | |
| N. crassa | RGGDWDKCFA⁵⁰⁹ | | | | |

Compared to the N. crassa putative phosphatase gene there are 197 basepair (bp) changes dispersed throughout the KPF0019-PHY gene, 7 codon deletions oriented toward the middle/end of the gene and a 1-codon insertion spanning bp 936-938 (KPF0019-PHY numbering). Table 6 is an alignment of the deduced amino acid sequences of the KPF0019-PHY and the N. crassa putative phosphatase genes. There are 42 amino acids changes between the two proteins. The deduced KPF0019-PHY protein sequence has one insertion at position 289 and 7 amino acid deletions clustered toward the C-terminal half of the protein, as compared to N. crassa putative phosphatase. In silico predicted biochemical properties of the KPF0019-PHY and N. crassa are shown in Table 7. Theoretically, these two proteins should have an almost identical biochemical profile. Together the sequence data indicates that strain KPF0019 is a close relative of N. crassa. However, the nucleotide and deduced amino acid sequence differences at the putative phytase/phosphatase loci provides sufficient evidence to conclude that although KPF0019 is probably of the genus Neurospora, it is not likely the species crassa.

TABLE 7
In silico analysis of the biochemical properties of the KPF0019
putative phytase versus the N. crassa putative phosphatase.

| | | N. crassa |
|---|---|---|
| Biochemical Properties | KPF0019-P | putative phosphatase |
| Length | 503 aa | 509 aa |
| Molecular Weight | 55198.19 | 55823.54 |
| 1 microgram = | 18.117 pMoles | 17.914 pMoles |
| Molar Extinction coefficient | 47000 | 47000 |
| 1 A[280] correlates to | 1.17 mg/ml | 1.19 mg/ml |
| Isoelectric point | 4.90 | 4.70 |
| Charge at pH 7 | −16.74 | −22.73 |

### EXAMPLE 3—EXPRESSION AND FURTHER BIOCHEMICAL CHARACTERIZATION OF PHYTASE FROM FUNGAL STRAIN KPF0019

Introduction. Media optimization was used to increase phytase expression from strain KPF0019 by 9-fold in liquid shake flask fermentations. The culture broth from KPF0019 was tested for its biochemical properties including pH and temperature activity profiles and pH and temperature stabilities. Data showed optimal pH at 5.5 and optimal temperature at 55° C. for both the phytase. The culture broth retained 40% phytase activity at 80° C. during temperature stability experiments and 60% phytase activity during pH stability experiments at pH 3 and 7.5.

Before biochemical properties could be determined for spent culture broth, media optimization was necessary to increase the levels of phytase expression by strain KPF0019. It has been shown that physical parameters influencing growth of an organism and its production of metabolites or protein are pH, temperature, agitation, dissolved oxygen, and pressure, while nutritional parameters such as carbon source, nitrogen source, trace elements, and vitamins affect the production of many phytases (13). Therefore, a wide variety of complex media along with one minimal media were tested for their effects on phytase expression from KPF0019. It has been shown that two surfactants, sodium oleate and Tween 80, can increase enzyme production and secretion in solid state and liquid fermentations (13, 14, 15, 16). Therefore, 0.1% sodium oleate and 0.5% Tween 80 were added to selected media. This Example provides information on the effects of surfactants, glycerol concentration, and temperature on increased production of phytase by strain KPF0019 in liquid shake flask fermentation. Additionally, biochemical properties (optimal temperature, temperature stability, optimal pH and pH stability) were determined for the KPF0019 phytase in the culture broth.

A. Materials and Methods

Materials. Tween 80 and rice phytic acid were purchased from Sigma. Aquacide II was purchased from Calbiochem. All other chemicals and buffers were of analytical reagent grade from Fisher.

Microorganism, media and conditions of growth. The microorganism was maintained on ISP2 solid medium composed of 1% malt extract, 0.5% yeast extract, 0.5% dextrose, 0.01% instant ocean salt, 1% potato flour, 2% agar and milli Q water. The microorganisms were inoculated after media cooling and incubated at 30° C. After 4 days, mycelia were formed and agar plates were stored at room temperature until use.

Expression of secreted phytase was evaluated by growing strain KPF0019 in K3 media (1.0 g/L peptonized milk, 1.0 g/L tryptone, and 5.0 g/L glucose), K5 media (8.0 g/L nutrient broth and 10 g/L glycerol) with or without 0.1% sodium oleate and 0.5% Tween 80, K4 media (35 g/L Czapek-Dox), K2 media (5 g/L tryptone, 3 g/L malt extract, 10 g/L dextrose, 3 g/L yeast extract), or M5 media (1.8 mL/L 5N NaOH, 20 g/L glucose, 1 mL/L K₂HPO₄, 12.6 mL/L N₂H₈SO₄, 2.7 mL/L 2M CaCl₂, 2.5 mL/L 2M MgSO₄, 1 mL/L 1000× trace mineral mix, 0.66 g/mL phytic acid from corn). Additional media used for the production of phytase from KPF0019 were Gaugy media (40 g/L glucose, 3 g/L NaNO₃, 2 g/L yeast extract, 1 g/L KH₂PO₄, 0.5 g/L KCL, 0.5 g/L MgSO₄*7H₂O), 10 mg/mL FeSO₄*7H₂O), Production media (PM) (1.4 g/L N₂H₈SO₄, 2.0 g/L KH₂PO₄, 0.3 g/L urea, 0.3 g/L MgSO₄*7H₂O, 0.005 g/L FeSO₄*7H₂O, 0.0016 g/L MnSO₄*H₂O, 0.0014 g/L ZnSO₄*7H₂O, 0.002 g/L COCl₂*6H₂O, 1 g/L pharmamedia, 2 g/L Tween 80, 11 g/L lactose, 5 g/L corn steep liquor powder, 0.3 g/L CaCl₂, 5.0 g/L nutrisoy), or corn starch (CS) media (PM with 40 g/L cornstarch). The inoculum size was 3-4 core plugs that were cultured in the different media for 7-9 days with shaking at 200 rpms at 29-34° C. Biomass and culture broth were separated by centrifugation or filtration through Whatman filter paper #2.

Phytase and protein determination. Analysis of samples for phytase activity was performed following the phytase assay described in Example 1. The assay was altered for each of the biochemical tests. The pH profiles were determined by measuring phytase activity with phytic acid at pH's between 2.5-8.5 at 37° C. for 60-180 minutes. Formic acid buffers, 0.1M (pH 2.5-3.5); acetate buffers, 0.1M (pH 4.0-5.5); Bis-Tris buffers, 0.1M (pH 6.0-7.0); and Tris-HCl buffers, 0.1M (pH 7.5-8.5) were used to achieve desired pH. Temperature profiles were determined by assaying phytase activity of the samples between 25-100° C. for 60-90 minutes with a final concentration of 5 mM phytic acid at pH 5.5. The pH stability experiments were performed by adjusting enzyme samples to pH 3.0, pH 5.5 or pH 7.0 with acid or base and then incubating for 24 hours at 4° C. or 25° C. then measuring phytase activity at pH 5.5. Temperature stability was determined by subjecting samples to 4° C. (control) or 30-100° C. for 20-30 minutes, followed by cooling on ice. Enzyme samples were then assayed for phytase activity in the standard assay at 37° C. Sample analysis for protein content was based on the Bradford assay and Coomassie Plus reagent (Pierce).

KPF0019 Phytase Culture Broth. The culture broth supernatant was stored at 4° C. and designated as the “KPF0019 broth”. Three lots of KPF0019 phytase were grown in shake flask fermentations as described above and employed for the biochemical characterization: lot 262-192 grown in K3 media, lot 297-124 grown in K5 media and lot 369-55 grown in K5 with 1% glycerol and 0.5% Tween 80. Each figure describing the culture broth phytase contains the mean of two lots. Not all data points have the same number of replicates.

B. Results and Discussion

Effect of different growth media conditions on secretion of phytase from KPF0019. Strain KPF0019 expressed different levels of phytase activity when grown in different media (Table 8). 0.025 U/mL and 0.034 U/mL of phytase activity were produced in K5 and K3 media, respectively. Levels of phytase activity less than 0.025 U/mL were expressed in complex media such as PM, CS, Gaugy's, and K2 media. Literature has shown that other phytase-producing microorganisms can be induced to express phytase by addition of phytic acid. However, induction of phytase expression by phytic acid was not observed with KPF0019 (Medium M5, Table 8).

TABLE 8
KPF0019 Phytase Expression on Defined Growth Media

| | Media¹ | | | | | | | |
|---|---|---|---|---|---|---|---|---|
| | M5 | K2 | K3 | K4 | CS | Gaugy | PM | K5 |
| Phytase | 0.003 | 0.000 | 0.034 | 0.010 | 0.004 | 0.000 | 0.009 | 0.025 |
| activity | | | | | | | | |
| (U/ml)² | | | | | | | | |

¹Grown at 29° C.
²Data shown are the mean of multiple replicates on a single growth experiment

Next, the effect of addition of surfactant to the media on secretion of phytase from KPF0019 was ascertained. As seen in Table 9, a 2.9 to 4.2-fold improvement in phytase activity was observed when strain KPF0019 was grown in K5 media containing Tween 80 or sodium oleate when compared to phytase activity expressed in the control media without surfactants. The addition of sodium oleate to K3 media increased the expression the level of phytase expression by 1.6 fold, but decreased expression was observed when Tween 80 was employed. Overall, K5 media containing Tween 80 showed the best expression of phytase in comparison with other media tested. Therefore, all further experiments to improve phytase expression were derived from K5 media with Tween 80.

TABLE 9
Effect of surfactants on phytase expression by KPF0019

| | Media¹ | | | | | |
|---|---|---|---|---|---|---|
| | K3 | K3-tw80 | K3-Na—O | K5 | K5-tw80 | K5-Na—O |
| Phytase activity (U/ml)² | 0.034 | 0.012 | 0.055 | 0.025 | 0.106 | 0.074 |

¹Grown at 29° C.
²Data shown are multiple replicates on a single growth experiment

The effects of temperature on phytase expression by KPF0019 are shown in Table 10. The optimal growth temperature for phytase expression from KPF0019 in shake flask fermentation using K5 with Tween 80 was between 32° C. and 35° C., resulting in 2-fold increase in phytase activity when compared to KPF0019 grown at 28° C.

TABLE 10
Effect of growth temperature on KPF0019 phytase expression

Temperature (° C.)

| | 28 | 32 | 35 | 37 |
|---|---|---|---|---|
| Phytase activity U/mL¹ | 0.106 | 0.225 | 0.234 | 0.105 |

¹Data shown are the mean of multiple replicates on a single growth experiment

Table 11 lists the effect of glycerol concentration in K5 media on KPF0019 phytase expression. The concentration of glycerol found to be optimal for phytase expression was 1%, the standard concentration of glycerol used in this medium. When glycerol was removed from the media, phytase expression decreased even though no visual decrease in biomass was detected. When glycerol levels reached 20% very little growth was observed and no phytase expression was detected. This high concentration of glycerol may have a physical effect on multiple cellular functions that clearly affect growth of the cells. In addition, KPF0019 mycelium cultivated on ISP2 agar for 4 days expressed higher levels of phytase activity than older mycelium (2-3 weeks old) (data not shown). This suggests that younger cells which are rapidly multiplying may express or secrete more phytase.

TABLE 11
Effect of glycerol concentration on KPF0019 phytase expression

| Percent of | Phytase | Protein | Specific activity |
|---|---|---|---|
| Glycerol² | U/ml | [mg/mL] | Unit/mg protein |
| 0 | 0 | N/a¹ | — |
| 1 | 0.320 | 0.166 | 1.93 |
| 5 | 0.016 | 0.352 | 0.330 |
| 10 | 0.044 | 0.424 | 0.104 |
| 20 | 0 | N/a | — |

¹N/a Not assayed
²K5 media at 34° C.

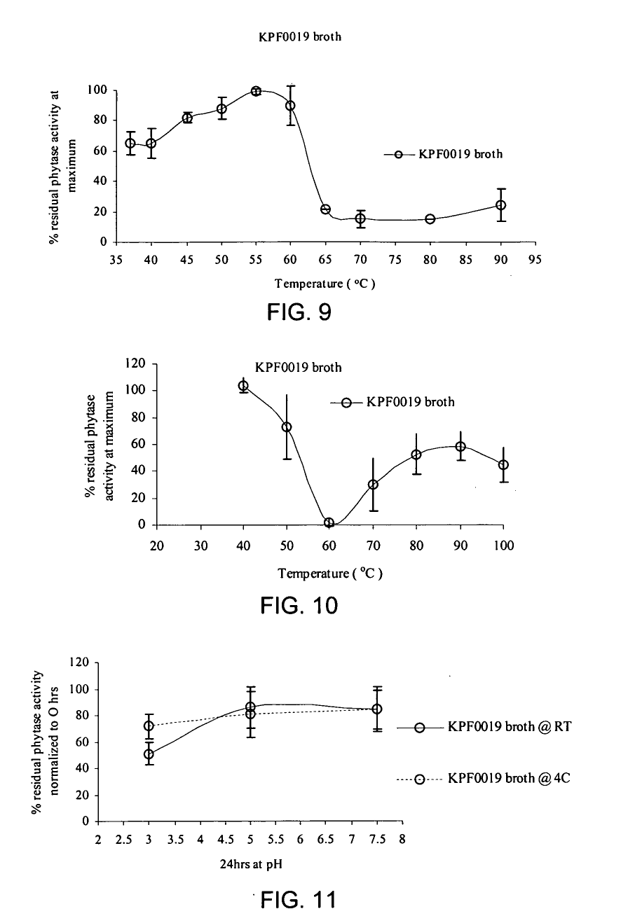

Biochemical properties of KPF0019 phytase from spent culture broth. The KPF0019 phytase in spent culture broth was evaluated to determine its biochemical properties. pH and temperature activity profiles for KPF0019 phytase in culture broth are shown in FIGS. 8 and 9, respectively. The optimal pH was about 5.5 with 60% phytase activity remaining at pH 4 and 6.5. The optimal temperature was about 55° C. A sharp decline in activity was observed for culture broth between 60-65° C. with 10 to 20% of phytase activity remaining between 70° C. and 90° C. (FIG. 9).

As seen in FIG. 10, phytase from KPF0019 spent culture broth exhibits an interesting temperature stability profile, with activity falling to zero with treatment at 60° C. for 30 minutes, but then the activity recovering to 50-60% of maximum with treatment at 80-100° C.

Three pH conditions were selected to determine the pH stability of KPF0019 culture broth. These pH ranges were based on the pH conditions in the digestive tract of monogastric animals, which can range from pH 3 to 7. As shown in FIG. 11, the pH stability of KPF0019 phytase from spent culture broth was greater than 60% under all experimental conditions, when normalized to the control at zero hours at the corresponding pH when assayed at 37° C.

Summary. It has been shown that several media components affect phytase expression by the fungal organism KPF0019. The addition of the surfactant Tween 80 to K5 medium resulted in greater enzyme production from KPF0019 than the addition of sodium oleate. It is unclear why Tween 80 and sodium oleate resulted in different levels of phytase expression, but it may be due to their different chemical structures. One percent glycerol as the carbon source resulted in the best phytase production, while no phytase activity was observed when glycerol was removed from the medium. Furthermore, by using 1% glycerol, young mycelium, and Tween 80, we were able to improve the expression levels by 9 fold. We also observed that KPF0019 strain could express phytase in both a complete media (K5) and a minimal media (K3).

pH and temperature profiles and pH and temperature stability, were determined. Optimal pH ranges similar to KPF0019 phytase (pH optimum 5-6) have been reported for some commercial phytase products: Natuphos, (pH 5-5.5), Ronozyme (pH 4.5), Finase (pH 5.5-6.0).

In the temperature stability experiment, less than 20% activity was observed at 55-60° C. for the KPF0019 phytase broth, but in the temperature activity profile experiment there was greater than 90% activity seen at the same temperature. This discrepancy is likely due to difference in the experimental designs since the temperature profile experiment required incubating enzyme sample and substrate for 1 hr at 55° C. while the temperature stability experiment incubated enzyme sample at 55-60° C., cooled the sample on ice for 5 minutes, and then assayed for activity with a 1 hour incubation at 37° C. The phytate could have an effect on the enzyme stability at higher temperatures, accounting for the different results observed at 55-60° C. in the two experiments.

KPF0019 phytase in spent culture is unique because of its observed activity when treated at 70° C.-100° C. for 30 minutes. This activity seen after treatment at elevated temperatures occurs despite the fact that treatment of the broth at 60° C. for 30 minutes results in a complete loss of activity. The reason for this phenomenon is unclear but may be due to a refolding event similar to that observed with phytase from Aspergillus fumigatus (17). This activity after treatment at higher temperatures also suggests that KPF0019 phytase may retain higher phytase activity after pelleting.

Some of the changes to the physical and nutritional parameters of media that result in an increase in phytase expression and/or secretion by KPF0019 are described. Four biochemical properties were also determined for the phytase in the culture broth from KPF0019.

### EXAMPLE 4—THERMOSTABILITY OF A NOVEL SECRETED PHYTASE FROM STRAIN KPF0019 USING AN IN VITRO FEED MATRIX SYSTEM

This Example contains additional biochemical data describing the stability of the KPF0019 phytase on a feed matrix system during heat treatments that were meant to mimic pelleting conditions. Steam pelleting would be the most favorable way to examine thermostability of the KPF0019 phytase. Alternatively, simulating pelleting by passing wet steam through feed can be used for examining KPF0019 phytase thermostability. However, we were unable to simulate pelleting conditions using either of these methods due to low expression levels of phytase from the KPF0019 strain. Therefore, an alternative method was devised in an attempt to determine the thermostability of KPF0019 phytase using a feed matrix system.

The KPF0019 phytase has been subjected to an in vitro feed matrix system for the measurement of phytase activity. The detection of phytase was based on the production of phosphate from the enzymatic hydrolysis of either pure rice phytic acid or natural phytate from the feed. Since extraction of KPF0019 phytase from the feed for subsequent hydrolysis of pure rice phytic acid was less than optimal, hydrolysis of natural phytate from the feed was used to compare the relative phytase thermostabilities.

Materials and Methods

Materials. Tween 80 and rice phytic acid were purchased from Sigma. Aquacide II was purchased from Calbiochem. All other chemicals and buffers were of analytical reagent grade from Fisher. Pelleted feed was obtained from a commercial broiler facility.

Preparation of KPF0019 phytase enzyme. From an agar plate, four plugs of KPF0019 were aseptically added to a 250 mL Erlenmeyer flask containing 50 mL of culture media (100 ml/L glycerol, 8 g/L nutrient both and 5 g/L Tween 80). The culture was grown for 7 days at 32-36° C. with shaking at 200 rpms and then filtered through a Whatman #2 filter paper to remove biomass. KPF0019 phytase in the broth was concentrated using ammonium sulfate precipitation. KPF0019 culture was gently mixed with 100% ammonium sulfate solution, pH 7.0, to 70% saturation and stored on ice for 30 minutes. After the material was centrifuged for 1 hr at 9000 rpms at 4° C., the supernatant was decanted. The pellet was then re-suspended in 21.5 mL of 0.01 M acetate buffer, pH 5.5, and centrifuged for 1 hr at 20,000 rpm at 4° C. to remove insoluble material. The phytase sample was then desalted through a gravity feed PD-10 Pharmacia column packed with Sephadex G-25M and eluted with 0.01 M acetate buffer, pH5.5. Phytase protein was further concentrated by placing the material into a 10,000 MWCO SnakeSkin pleated dialysis tubing (Pierce) and covered with Aquacide II at 4° C. In this manner, 480 mL of KPF0019 broth containing 0.2 U/mL phytase activity was concentrated to 7 mL containing 10.27 U/mL phytase activity. This represents 75% recovery of phytase activity. This concentrated material was used for all experiments in this Example.

Application of phytase to feed. Pelleted feed of a typical corn and soy-based broiler finisher diet was ground to pass a 2 mm screen and 5 g samples were aliquoted into Erlenmeyer flasks. When necessary, dilution of the enzyme was made in 0.01 M acetate buffer, pH 5.5 and applied drop-wise onto the feed and swirled gently to coat the feed. The KPF0019 phytase was applied at an equivalent of 500 U/kg feed. To allow sufficient contact time, the feed and enzyme were stored overnight at room temperature in covered flasks. Each experiment was performed with duplicate flasks of the treatment and control. The treatment is defined as phytase applied to feed with heat while the control was phytase applied to feed without heat.

Thermostability of phytase in an in vitro feed matrix. The 5 grams of feed had, based on information from the manufacturer, approximately 5% moisture content. To mimic the typical 18% moisture content of feed during pelleting, 0.4 mL of enzyme or water were added to the feed in a 250 mL Erlenmeyer flask, raising the moisture content an additional 13%. The flask was then capped and the feed and enzyme or water mixture was placed into a shallow water bath for 1 to 15 minutes at 75° C. or 90° C. After heat treatment, samples were cooled on ice for 5 minutes. The remaining phytase activity was measured two different ways. In the first set of experiments, 25 mL of 0.01 M acetate buffer, pH 5.5 was used to extract phytase from the feed and then extracted phytase was filtered through Whatman #2 filter paper. The filtrate was assayed for phytase activity using the standard phytase microplate assay described in Example 1. In the second set of experiments, 25 mL of 0.01 M acetate buffer, pH 5.5 was added to the feed and enzyme or water mixture to form a slurry which was allowed to incubate at 37° C. for 30 minutes at 200 rpm using the phytate in the feed as substrate. After the reaction was completed, the slurry was filtered through Whatman #2 filter paper. The filtrate was measured for release of phosphate using Fiske and Subbarrow reducer. Phytase activity in each flask was calculated by subtracting the phosphate released from the background phosphate measured in control flasks without enzyme.

B. Results and Discussion

Determination of phytase thermostability using phytase extracted from feed matrix. Pelleted feed was selected over mash feed for all studies since less phosphate release was previously observed using the pelleted feed. To determine phytase thermostability of KPF0019 phytase extracted from the feed, duplicate or triplicate flasks were employed and KPF0019 phytase was applied to ground pelleted feed. The phytase was subsequently extracted from the feed using extraction conditions described in the materials and methods. Only 0.006 U/mL was extracted from the feed for the KPF0019 phytase, which represents only a 24% recovery. This low recovery of KPF0019 phytase from the feed may be due to the binding of phytase protein to the feed matrix under these conditions.

Determination of phytase thermostability using feed as substrate. To address the concern of low recovery of phytase from the feed, a series of experiments were performed to determine the activity of phytase using the phytate in the feed matrix as substrate. Heat treatment of KPF0019 phytase was subjected to 90° C. for 5 and 15 minutes, cooled, and allowed to incubate 30 minutes with feed at 37° C. Inactivation of commercially available phytases due to heating at 90° C. for 15 minutes showed similar results to those for the activities observed after pelleting (18, 19, 20). Using these conditions, 23.5% of KPF0019 phytase activity remained (data not shown).

Summary. The extraction of KPF0019 phytase from feed showed low extraction efficiencies, possibly because this unique phytase has higher binding affinity to the feed. This binding effect could likely be due to the wide variety of feed components, such as ground corn and soybean or available phytate. Additionally, KPF0019 phytase does not contain other stabilizing compounds such as sorbitol or propylene glycol, which are typically formulated into commercial products and may affect extraction from the feed. However, our data indicate the phytase is still active, as observed when the feed was subsequently used as substrate for KPF0019 phytase.

Our in vitro data demonstrate that the thermostability of KPF0019 phytase on feed is similar to the thermostability of the other commercially available phytases.

### EXAMPLE 5—EXPRESSION OF THE KPF0019 PHYTASE GENE IN Escherichia coli

A novel gene has been cloned from fungal strain KPF0019 that likely codes for a phytase enzyme. In this Example we show that this gene indeed codes for an active phytase enzyme and demonstrate heterologous production of a phytase enzyme product by over-expressing the KPF0019-Phy gene in Escherichia coli. The native KPF0019 phytase gene contains a 65-basepair intron and a secretion signal sequence in the 5′ region of the gene. Therefore, it was necessary to genetically engineer the gene and remove these sequences prior to cloning and cytoplasmic expression in E. coli. Two distinct genetic constructs were engineered; one where the phytase gene sequence begins at basepair 132 and another that begins at basepair 147 (numbering with respect to the native KPF0019 phytase gene start codon). These nucleotide positions correspond to the mature expressed proteins beginning with an artificial methionine followed by either amino acid 23 or 28 of the KPF0019 phytase, respectively. The genetically engineered KPF0019 gene constructs were transformed into the appropriate E. coli host strain and induced for over-expression. Induction of both of the engineered forms of the KPF0019 phytase gene resulted in the production of an active phytase enzyme.

To demonstrate that the putative KPF0019 phytase (KPF-phy) gene in fact codes for an active phytase enzyme, we engineered the native KPF-phy gene for expression in the microbial host Escherichia coli. The pET expression system was chosen to express the KPF-phy gene because it is a powerful system that has often been used to express a diverse assortment of recombinant prokaryotic and eukaryotic proteins in E. coli. Target genes are cloned into pET plasmids under control of the strong bacteriophage T7 transcription and translation signals where expression is induced by providing a source of T7 RNA polymerase in the host cell. T7 RNA polymerase is so selective that, when fully induced, almost all of the cell's resources are converted to target gene expression. The desired product can comprise more than 50% of the total cell protein a few hours after induction. Target genes are initially cloned using strains that do not contain the T7 RNA polymerase gene. This eliminates plasmid instability due to the production of proteins potentially toxic to E. coli. Once established in a non-expression host, target protein expression is initiated by transferring the plasmid into an expression host containing a chromosomal copy of the T7 RNA polymerase gene under control of the lacUV5 promoter. Expression of target genes in the pET system is under control of the T7lac promoter. pET plasmids contain a lac operator site just downstream of the T7 promoter. They also carry the natural promoter and coding sequence for the lac repressor (lacI) oriented so that the T7lac and lacI promoters diverge. When the pET vector is transformed into a host cell that is a DE3 lysogens, the lac repressor acts at both the lacUV5 promoter in the chromosome to repress transcription of the T7 RNA polymerase gene and the T7lac promoter to repress expression of the target gene. Only a few target genes have been encountered that are too toxic to be stable in these vectors.

This Example describes the genetic engineering and over-expression of the KPF-phy gene in E. coli using the commercially available pET expression system. Our results demonstrate that E. coli cells harboring the KPF-phy gene produce phytase activity, whereas cell without the KPF-phy gene do not. This provides direct evidence that the gene cloned from KPF0019 codes for a phytase enzyme.

A. Experimental Procedures

Strains and Media. Genotypes of strains and plasmids used in this study are listed in Table 12. Escherichia coli XL1-Blue MRF′ (Stratagene, La Jolla, Calif.) was used for general cloning purposes. E. coli strain BL21(DE3) (Novagen, Madison, Wis.) was used as the host for protein expression. Bacterial strains were grown in either Luria-Burtani (LB) broth (per liter: Bacto tryptone, 10 g; Bacto yeast extract, 5 g; NaCl, 10 g) or on LB agar (LB broth plus 1.5% agar). For plasmid maintenance, ampicillin (75-100 μg/ml) was added to LB broth and LB agar when needed.

TABLE 12
Escherichia coli strains and plasmids used in Example 5

| Strain or | | |
|---|---|---|
| plasmid | Relevant genotype or description | Source or reference |
| Strains | | |
| XL1-Blue | Δ (mcrA)183 Δ (mcrCB-hsdSMR- | Stratagene, |
| MRF' | mrr)173 endA1 supE44 thi-1 | La Jolla, CA |
| | recA1 gyrA96 relA1 lac [F′ | |
| | proAB lacI^{q}Z D M15 Tn10 (Tet^{r})] | |
| BL21(DE3) | F⁻ ompT hsdS_{B} (r_{B}⁻ m_{B}⁻) gal dcm | Novagen, |
| | (DE3) | Madison, WI |
| Plasmids | | |
| pEcPh-1 | Full length KPF0019 phytase gene | Example 2 |
| | in plasmid pCR ® 2.1-TOPO ® | |
| pET-25b(+) | F′, amp^{r} T7lac, pelB signal | Novagen, |
| | sequence, C-terminal HSV•Tag ® | Madison, WI |
| | and His•Tag ® | |
| pEcPh-23 | KPF0019 gene starting at bp 132 | Example 5 |
| | in pET-25b(+) | |
| pEcPh-28 | KPF0019 gene starting at bp 147 | Example 5 |
| | in pET-25b(+) | |

DNA manipulation. Isolation of plasmid DNA, restriction digestions, ligations, and plasmid transformations were performed as previously described (15, 11).

Cloning of the KPF0019 phytase gene into plasmid pET-25b(+). Two distinct genetic versions of the KPF-phy gene were created and cloned into the pET-25b(+) plasmid. The gene constructs are modified versions of the wildtype KPF-phy gene and were created via PCR amplification. The gene constructs created for expression in E. coli differ from the wildtype gene in that each is truncated in the 5′ region. One construct begins at begins at basepair (bp) 132 and another begins at bp 147 (numbering with respect to the native KPF-phy start codon). These nucleotide positions correspond to codons 23 and 28, respectively, of the wildtype KPF-phy gene. The gene constructs were designed to be expressed cytoplasmically in E. coli and therefore part of the construction required the addition of an artificial start codon immediately adjacent to either codon 23 or 28. Oligonucleotide primers were designed to create in-frame translational fusions with the T7lac promoter, including the ATG start codon, in the pPET-25b(+) plasmid (FIG. 1). Integrated DNA Technologies, Inc. (Iowa City, Iowa) synthesized all primers. Plasmid pEcPh-23 was created by amplifying a 1536 bp region of the wildtype KPF-phy gene using the upstream primer EcoF-23 (5′-GGAATTCCATATGCAACCAGTCCCATGCGAC-3′) in combination with the downstream primer M13 Reverse (−27) (5′-GGAAACAGCTATGACCATG-3′). The 5′ end of EcoF-23 contains an artificial Nde I site (which contains an artificial ATG start codon sequence) followed by nucleotide sequence complementary to the KPF-phy gene starting at nucleotide 132. The M13 Reverse (−27) primer is downstream of the 3′ end of the KPF-phy gene stop codon and complementary to template DNA in pEcPh-1 downstream of an EcoR I site. Using M13 Reverse (−27) adds an additional 90 nucleotides to the 3′ end of the amplified fragment (included in the 1536 bp above). Each 50 μl PCR reaction mixture contained approximately 10 ng pEcPh-1 template DNA, 500 nM of each primer, 200 μM dNTPs, 1×PFU Turbo Buffer (Stratagene, La Jolla, Calif.) and 2.5 U PFU Turbo Polymerase (Stratagene). The thermocycling program included one cycle at 95° C. (5 min) and 35 cycles of 95° C. (30 s), 60° C. (1 min) and 72° C. (1.5 min) immediately followed by 72° C. (10 min) and an indefinite hold at 4° C. Amplified PCR products were visualized by electrophoresis through a 1% agarose gel containing 0.1 μg/mL ethidium bromide. Gel slices containing the expected sized bands were excised and the DNA was eluted using the Qiagen Gel Extraction Kit (Qiagen, Valencia, Calif.). PCR products were digested with EcoR I and Nde I, visualized and purified as described above. The digested PCR product was ligated into the EcoR I-Nde I sites of plasmid pET-25b(+) and transformed into E. coli XL1-Blue MRF′. The sequence the KPF-phy gene insert in pEcPh-23 was confirmed by DNA sequencing performed at the Iowa State University DNA Sequencing and Synthesis Facility (Ames, Iowa) using the dideoxy method via the ABI PRISM Dye Terminator Cycle Sequencing Kit (Applied Biosystems, Foster City, Calif.) and analysis with either the ABI Model 377 Prism DNA Sequencer or the ABI 3100 Genetic Analyzer (Applied Biosystems). Plasmid pEcPh-28 was constructed in the same manner as described above for pEcPh-23, except the upstream primer used to amplify the KPF-phy gene was oligonucleotide EcoF-28 (5′-GGAATTCCATATGGACACCCCCGAGCTTGGT-3′). The 5′ end of primer EcoF-28 contains an artificial Nde I site (which contains an artificial ATG start codon sequence) followed by nucleotide sequence complementary to the KPF-phy gene starting at nucleotide 147. The DNA sequence of pEcPh-28 was verified as stated above.

Induction of recombinant phytase expression in E. coli. Plasmids pEcPh-23, pEcPh-28, and pET-25b(+) (negative control) were transformed separately into expression host BL21 (DE3). A single colony from each transformation was used to inoculate 10 mL LB broth containing 100 μg/mL ampicillin and then grown overnight (12 h) at 37° C. with shaking at 250 rpm. Each culture was diluted 1:50 into 25 mL LB broth containing 50 μg/mL ampicillin and grown at 37° C. until the OD₆₀₀ reached 0.6. One mM IPTG was added to each culture and the cultures allowed to grow for an additional 4 hours at 29° C. Cells were harvested by centrifugation, frozen in liquid nitrogen, and stored at −20° C. until use.

Assay of recombinant phytase expression from E. coli. Cells from 24 mL of culture were resuspended in 2.5 mL sonication buffer (25 mM acetate, pH 6.0 containing 100 mg/L each PMSF (Sigma, St. Louis, Mo.) and benzamidine (ICN, Aurora, Ohio)). Cell lysis was accomplished by exposing each sample to four, 20 sec bursts of sonication, which included 20 sec intervals of chilling the cells on ice between bursts. A small volume (<200 μl) was removed from each sample and designated ‘total sonicate’. The remaining sample was centrifuged for five minutes at 13,000 rpm and the supernate removed to a new tube (‘supernatant sonicate’). Fifty μl of each sample was assayed for phytase enzyme activity using the microtiter plate method described in Example 1, with some minor modifications. The most notable modification was that each sonicated solution served as its own control. Controls consisted of addition of a TCA (final concentration was 2%) to each sample prior to phytate addition, followed by incubation at 37° C. for one hour.

SDS-PAGE analysis of E. coli cells expressing phytase. 25 μl of an SDS-gel loading buffer was added to 75 μl of each of the sonicates (total or supernatant), each sample was boiled for five minutes, and 20 μl was loaded onto an 8% SDS-PAGE gel. After electrophoresis, protein bands were visualized by staining with Gel Code Blue (Pierce, Rockford, Ill.).

B. Results and Discussion

Expression of the KPF-phy gene in E. coli. The native KPF-phy gene contains an intron and a signal sequence in the 5′ region of the gene. Since E. coli is a prokaryotic organism and the KPF-phy gene is from a eukaryotic organism, E. coli will not properly process either of these genetic regulatory elements. Therefore, the KPF-phy gene was genetically engineered and the native intron and signal sequences were removed prior to cloning into the pET expression plasmid. Two distinct genetic constructs were engineered; one where the phytase gene begins at bp 132 and another that begins at bp 147 (numbering with respect to the KPF-phy gene start codon). These nucleotide positions correspond to mature proteins beginning with an artificially engineered methionine followed by amino acids 23 and 28, respectively, of KPF0019 phytase. In the pET expression system regulation of target gene expression is under control of the T7lac promoter. Accordingly, each truncated KPF-phy gene construct, respectively, was cloned downstream of the T7lac promoter creating a translational fusion between the promoter and the engineered phytase gene, as described by Novagen in their product literature. The T7lac promoter-KPF-phy gene fusions were constructed in a non-expression host strain of E. coli (XL1-Blue MRF′) and then transformed into strain BL21 (DE3) for expression. E. coli strain BL21 (DE3) contains the T7 RNA Polymerase gene whose expression is under control of the isopropyl-β-D-thiogalactopyranoside (IPTG)-inducible lacUV5 promoter (also as described by Novagen). Addition of IPTG to growing cells derepresses the lacUV5 promoter and induces expression of T7 RNA Polymerase. This polymerase in turn drives expression of the T7 promoter fused to the KPF-phy gene in plasmids pEcPh-23 and pEcPh-28.

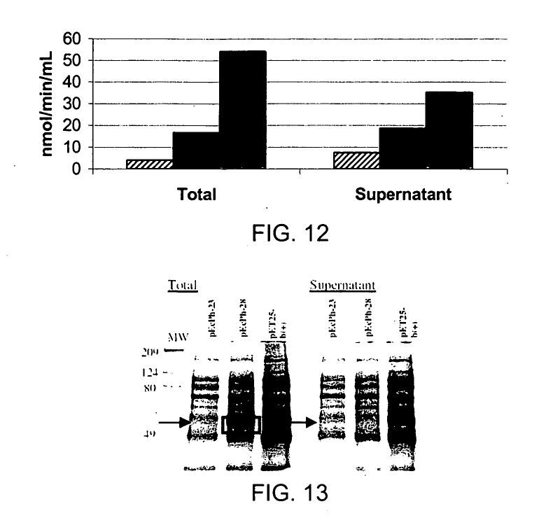

Plasmids pEcPh-23 and pEcPh-28 were designed to overproduce native phytase protein in the cytoplasm of BL21 (DE3). After IPTG induction cells were sonicated to release intracellular proteins. The sonicates were separated into 2 fractions, total sonicate and sonicate supernatant, and each fraction was analyzed for phytase activity. Phytase activity was observed in BL21(DE3) induced transformants containing either pEcPh-23 or pEcPh-28, but not pET25-b(+) (FIG. 12). The only difference between these transformants is the presence of the KPF-phy gene, indicating that the presence of the KPF-phy gene is responsible for the activity. This result provides direct evidence that the KPF-phy gene codes for a phytase. Overall, more phytase activity was produced from cells carrying pEcPh-28 and most of the activity was found in the total sonicate fraction. It is unclear as to why there is a difference in activity between the total sonicate and sonicate supernatant fractions. Transformants carrying pEcPh-23 also expressed measurable phytase activity after induction, but much less than the pEcPh-28 cells (FIG. 12). The activity profiles between the total and supernatant sonicate were the same for cells carrying pEcPh-23, indicating phytase was likely soluble and located in the cytoplasm. Sonicate fractions were also analyzed by SDS-PAGE. FIG. 13 shows the presence of a predominant band at the expected molecular weight (red arrow) for recombinant phytase in the pEcPh-28 total sonicate (red box), which is not present in the control pET25-b(+) total sonicate. This band is absent in the sonicate supernatant of pEcPh-28. Recombinant phytase expressed from pEcPh-23 is not visible in either fraction indicating that the difference in phytase activity (FIG. 12) between these two transformants may be due to a difference in expression level.

Summary. The data presented here clearly show that the gene cloned from KPF0019 codes for a phytase and provides a proof of concept for the heterologous expression of the KPF0019 phytase enzyme. Significant levels of phytase protein were expressed in E. coli (particularly when using the pEcPh-28 construct) and the enzyme produced retained its activity.

### EXAMPLE 6—EXPRESSION OF THE KPF0019 PHYTASE GENE IN Trichoderma reesei RUT-C30

In this Example we describe the use of the cellulolytic filamentous fungi Trichoderma reesei RUT-C30 as a host for the production of recombinant KPF0019 phytase. The KPF0019 phytase gene was fused to the T. reesei RUT-C30 cellobiohydrolase I secretion signal and fusion expression was driven by the cellobiohydrolase I gene promoter. Eight hundred and six transformants harboring the promoter-secretion signal-KP0019 phytase gene fusion were isolated and a fraction were screened for recombinant phytase production. Eight percent of the screened transformants secreted soluble and active recombinant KPF0019 phytase into the culture medium.

T. reesei is an attractive host for many different reasons including its hyper-secretory capacity, its GRAS status for feed enzyme production, easy and inexpensive to cultivate, and eukaryotic secretory machinery and protein modification systems (29, 30, 31). There are also significant disadvantages associated with using this organism including its slower growth rate, tedious genetic engineering techniques and screening campaigns to produce desired strains, and variable to low-level heterologous protein expression (29, 30, 31, 32, 33, 34, 35). Hyper-secretory mutants of T. reesei can produced up to 40 g secreted protein per liter culture broth and approximately half of this consists of the main cellulase, cellobiohydrolase I (CBHI) (33, 35). The strong, inducible cbhI promoter drives this high-level cellulase expression and it has been used extensively in a number of homologous and heterologous expression systems (33, 34, 35). The expression cassettes described in this paper utilized the strong cbhI promoter to drive expression of the KPF0019 phytase (KPF-phy) gene.

This Example describes the genetic engineering and expression of the KPF-phy gene in T. reesei RUT-C30 using the native cbhI promoter to drive its expression and the CBHI secretion signal to target it for secretion. Through a lengthy transformation and screening campaign we were able to isolate eight transformants that expressed phytase.

A. Materials and Methods

Strains, plasmids, and media. Plasmids and strains used in this study are listed in Table 13. Escherichia coli strain XL1-Blue MRF′ (Stratagene, LaJolla, Calif.) was grown in Luria-Burtani (LB) broth (per liter: Bacto tryptone, 10 g; Bacto yeast extract, 5 g; NaCl, 10 g) or on LB agar (low salt LB broth plus 1.5% Bacto agar) and supplemented with 50-100 μg/mL of ampicillin (Invitrogen, Carlsbad, Calif.) when used for propagation of recombinant plasmids. T. reesei RUT-C30 was grown at 29° C. on either V8 agar (per liter: 200 mL V8 juice (Campbell Soup Company, Camden, N.J.), 1.5 g CaCO₃ and 15 g Bacto agar) or potato dextrose agar (PDA) (potato dextrose broth plus 2% Bacto agar) (Difco, Detroit, Mich.). T. reesei RUT-C30 (KPF-phy) transformants were selected on PDA containing 100 ug/mL hygromycin B. For phytase assays, transformants were grown in production media (per liter: 1.4 g (NH₄)₂S0₄, 2 g KH₂P0₄, 0.3 g urea, 0.3 g MgS0₄.7H₂0, 5 mg FeSO₄.(7H₂O), 1.6 mg MnSO₄.(H₂O), 1.4 mg ZnSO₄.(7H₂O), 2 mg CoCl₂.(6H₂O), 1 g parmamedia, 2 g Tween 80, 11 g lactose, 5 g corn steep liquor powder, 0.3 g CaCl₂, 5 g soybean hulls, and 0.05 mg biotin).

TABLE 13
Strains and plasmids used in Example 6

| | | Source or |
|---|---|---|
| Strain or plasmid | Relevant genotype or description | reference |
| Strains | | |
| Escherichia coli | Δ (mcrA)183 Δ (mcrCB-hsdSMR- | Stratagene, |
| XL1-Blue MRF' | mrr)173 endA1 supE44 thi-1 | La Jolla, CA |
| | recA1 gyrA96 relA1 lac [F′ proAB | |
| | lacI^{q}Z D M15 Tn10 (Tet^{r})] | |
| Trichoderma | Hyper-secretor, ATCC 56765 | ATCC, |
| reesei RUT-C30 | | Manassas, VA |
| Plasmids | | |
| pEcPh-1 | KPF-phy::pCR ® 2.1-TOPO ® | Example 2 |
| pTrPh-23 | KPF-phy (starting at | Example 6 |
| | bp 132)::pTrPI-20 | |
| pTrPh-28 | KPF-phy (starting at bp | Example 6 |
| | 147)::pTrPI-20 | |

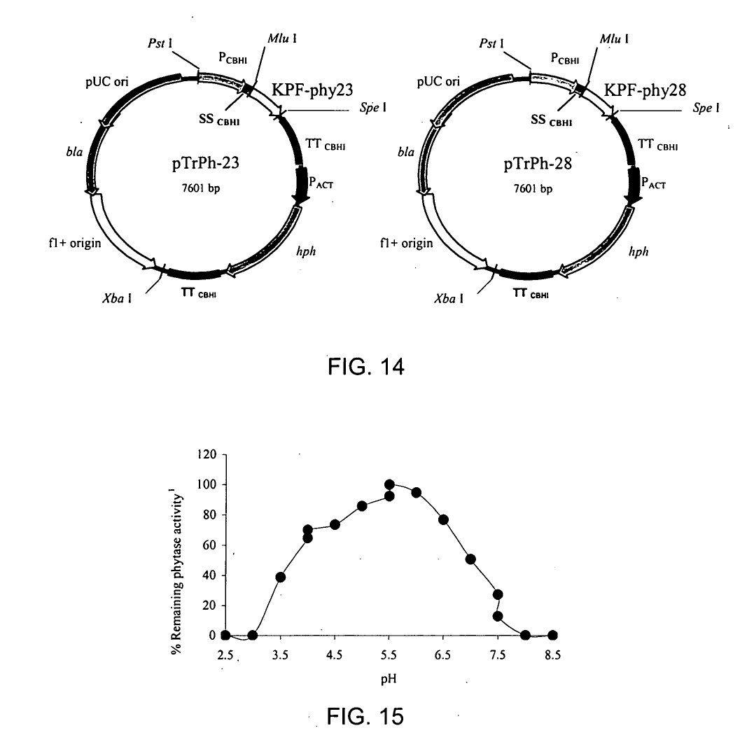

Cloning of the KPF0019 phytase gene into a Trichoderma expression vector. Genetic constructs were created and propagated in E. coli XL1-Blue MRF′ (28, 36). Two distinct genetic versions of the KPF-phy gene were created and cloned into a Trichoderma expression vector (FIG. 14). The gene constructs were created via PCR amplification and are modified versions of the wildtype KPF-phy gene, in that each is truncated in the 5′ region. One construct begins at begins at basepair (bp) 132 and another begins at bp 147 (numbering with respect to the native KPF-phy start codon). These nucleotide positions correspond to codons 23 and 28, respectively, of the wildtype KPF-phy gene. The gene constructs were designed for recombinant phytase (rPhy) to be expressed and secreted from T. reesei Rut-C30, therefore oligonucleotide primers were designed to create in-frame translational fusions with the CBHI secretion signal present in pTrPI-20. Integrated DNA Technologies, Inc. (Iowa City, Iowa) synthesized all primers. Plasmid pTrPh-23 was created by amplifying a 1536 bp region of the wildtype KPF-phy gene using the upstream primer TriF-23 (5′-CGACGCGTCAACCAGTCCCATGCGAC-3′) in combination with the downstream primer M13 Reverse (−27) (5′-GGAAACAGCTATGACCATG-3′). The 5′ end of TriF-23 contains an artificial Mlu I site followed by nucleotide sequence complementary to the KPF-phy gene starting at nucleotide 132. The M13 Reverse (−27) primer is downstream of the 3′ end of the KPF-phy gene stop codon and complementary to template DNA in pEcPh-1 downstream of an EcoR I site. Using M13 Reverse (−27) adds an additional 90 nucleotides to the 3′ end of the amplified fragment (included in the 1536 bp above) and includes an Spe I site. Each 50 μl PCR reaction mixture contained approximately 10 ng pEcPh-1 template DNA, 500 nM of each primer, 200 μM dNTPs, 1×PFU Turbo Buffer (Stratagene) and 2.5 U PFU Turbo Polymerase (Stratagene). The thermocycling program included one cycle at 95° C. (5 min) and 35 cycles of 95° C. (30 s), 60° C. (1 min) and 72° C. (1.5 min) immediately followed by 72° C. (10 min) and an indefinite hold at 4° C. Amplified PCR-products were visualized by electrophoresis through a 1% agarose gel containing 0.1 μg/mL ethidium bromide. Gel slices containing the expected sized bands were excised and the DNA was eluted using the Qiagen Gel Extraction Kit (Qiagen, Valencia, Calif.). PCR products were digested with Mlu I and Spe I, visualized and purified as described above. The digested PCR product was ligated into the Mlu I-Spe I sites of a Trichoderma expression vector (FIG. 14) and transformed into E. coli XL1-Blue MRF′. The sequence the KPF-phy gene insert in pTrPh-23 was confirmed by DNA sequencing performed at the Iowa State University DNA Sequencing and Synthesis Facility (Ames, Iowa) using the dideoxy method via the ABI PRISM Dye Terminator Cycle Sequencing Kit (Applied Biosystems, Foster City, Calif.) and analysis with either the ABI Model 377 Prism DNA Sequencer or the ABI 3100 Genetic Analyzer (Applied Biosystems). Plasmid pTrPh-28 was constructed in the same manner as described above for pTrPh-23, except the upstream primer used to amplify the KPF-phy gene was oligonucleotide TriF-28 (5′-CGACGCGGACACCCCCGAGCTTGGT-3′). The 5′ end of primer TriF-28 contains an artificial Mlu I site followed by nucleotide sequence complementary to the KPF-phy gene starting at nucleotide 147. The DNA sequence of pTrPh-28 was verified as stated above.

T. reesei RUT-C30 transformation, and culture-tube and shake-flask expression of recombinant KPF0019 phytase gene. Conidial spores of T. reesei RUT-C30 were harvested from 10-14 day old plates of either V8 or PDA by adding 5.5 mL sterile dH₂O to the plate and gently rubbing with a bent glass rod. Conidia were diluted 1000-fold and counted using a hemocytometer. Conidia were collected by centrifugation at 7,000 rpm for 10 min then washed two times with 10 mL ice-cold 1.2 M sorbitol. Conidia were resuspended to a final concentration of 2.5×10⁹ conidia/mL in 1 M sorbitol. Ten μg of expression cassette DNA (Pst I-Xba I fragments) (FIG. 1) from either plasmid pTrPh-23 or pTrPh-28, respectively, was mixed with 40 μl of conidia and transformed by electroporation (1.5 kV, 50 μF, and 300Ω). Immediately following electroporation 1 mL of 1 M sorbitol was added, conidia were plated on PDA plus 100 μg/mL hygromycin B (PDA-H), and incubated for 5-10 days at 30° C. To determine transformant stability resultant colonies were first passed to PDA (without antibiotic) and grown for 5-7 days at 30° C. Transformants were then passed from PDA to PDA-H and grown at 30° C. for 5-7 days. Transfromants that survived this passage were analyzed for phytase expression.

Hygromycin B-resistant (hyg^{R}) transformants of T. reesei RUT-C30 were inoculated into 50 mL glass tubes containing 5 mL of production media and grown for 7 days at 30° C. with shaking at 200 rpm. Biomass was removed by centrifugation and an aliquot of each supernatant was assayed for phytase activity using the microtiter plate method described in Example 1, with some minor modifications. The most notable modification was that each sample served as its own control. Controls consisted of addition of TCA to each sample prior to phytate addition, followed by incubation at 37° C. for one hour. Based on the results of the phytase activity, one transformant (TrPh-150) was chosen for further study. To confirm phytase expression, transformant TrPh-150 was streaked for single colony isolation on V8 agar, a single colony was picked, and grown in a 250 mL erlenmeyer flask containing 50 mL of inoculum media (per liter: 1.4 g (NH₄)₂S0₄, 2 g KH₂P0₄, 0.3 g urea, 0.3 g MgS0₄.(7H₂0), 5 mg FeSO₄.(7H₂O), 1.6 mg MnSO₄.(H₂O), 1.4 mg ZnSO₄.(7H₂O), 2 mg CoCl₂.(6H₂O), 1 g parmamedia, 0.75 g peptone, 2 g Tween 80, and 10 g glucose) for 72 hours at 30° C. at 200 rpms. After 72 hours, 2.5-5.0 mL of growth was transferred to a 250 mL Erlenmeyer flask containing 50 mL of fresh production media and grown for an additional 7 days at 30° C. at 200 rpm. Biomass was removed by centrifugation at 18,000 rpm for 30 min at 10° C., the supernatant was transferred to a sterile 50 mL conical tube, and stored at 4° C. until use. Biomass was stored in NTG (per liter: 8 g NaCl, 0.25 g Tween 80, and 200 mL glycerol) at −20° C.

Biochemical methods. Biochemical analyses were conducted on the recombinant KPF0019 phytase gene (rPhy) present in the spent culture broth of transformant TrPh-150. The pH profile of rPhy was determined by first adjusting enzyme samples to pHs between 2.5-8.5 using various buffering systems (0.1 M formate, pH 2.5-3.5; 0.1 M acetate, pH 4.0-5.5; 0.1 M Bis-Tris, pH 6.0-7.0; 0.1 M Tris-HCl, pH 7.5-8.5). Then five mM phytic acid (at the same pH as the sample) was added and the samples were incubated at 37° C. for 60 min. Following incubation, phytase activity was measured using the microtiter plate method described in Example 1 with minor modifications, as stated above. The temperature profile of rPhy was determined by heating enzyme samples with 5 mM phytic acid at temperatures between 25-100° C. for 60 min followed by measurement of phosphate released. The pH stability profile of rPhy was determined by adjusting the pH of enzyme samples to between pH 3.0 and 8.0 followed by 24 h incubation at 4° C. and 25° C., respectively. After 24 h, samples were adjusted to pH 5.5 and phytase activity was determined. The temperature stability of rPhy was determined by subjecting enzyme samples to various temperatures (between 30-100° C.) for 20 minutes. After heating, samples were cooled on ice and assayed for phytase activity at 37° C.

B. Results and Discussion

Cloning the KPF0019 phytase gene into the Trichoderma expression vector and transformation of T. reesei RUT-C30. The aim of this study was to over-produce soluble and active phytase protein in T. reesei RUT-C30. In all organisms, secreted proteins are synthesized as preprotein precursors, which include an N-terminal signal peptide that targets them to a secretory pathway (31). It has been shown that T. reesei RUT-C30 processes native secretion signals more efficiently than foreign secretion signals. The CBHI preprotein contains an N-terminal 17 amino acid secretion signal, which includes a processing target consisting of a basic-hydrophobic amino acid sequence (RAQ), which is cleaved in a KEX-independent manner (29, 30, 31). Processing of this secretion signal is very effective as evidenced by CBHI representing greater than 40% of the total protein secreted by T. reesei RUT-C30 (29, 33, 35). The native KPF-phy gene contains an intron and a secretion signal sequence in the 5′ region of its DNA sequence. Therefore, the KPF-phy gene was genetically engineered to remove the intron and secretion signal sequence prior to cloning into the Trichoderma expression vector. To ensure proper processing and efficient secretion of rPhy, the KPF-phy gene was fused to the CBHI secretion signal sequence. Two distinct genetic constructs were engineered in which the KPF-phy gene was translationally fused downstream of the CBHI secretion signal; in pTrPh-23 the phytase gene begins at bp 132 and in pTrPh-28 the gene begins at bp 147 (numbering with respect to the KPF-phy gene start codon) (FIG. 14). These nucleotide positions correspond to the mature proteins beginning with either amino acid 23 or 28, respectively, of rPhy. The strong, inducible cbhI promoter is commonly used to drive expression of recombinant proteins in T. reesei RUT-C30 and has been used in tandem with the CBHI secretion signal to enhance expression and secretion of a number of proteins (32, 33, 34, 35, 37). Therefore, the cbhI promoter was chosen to drive expression of the CBHI secretion signal-KPF-phy fusion. In order to increase transformation efficiency, the expression cassettes from plasmids pTrPh-23 and pTrPh-28 were first removed by restriction endonuclease digestion with Xba I and Pst I prior to electroporation into T. reesei RUT-C30. Since the expression cassettes lack an origin of replication, they cannot autonomously replicate in T. reesei RUT-C30. Therefore, the recovery of hyg^{R} transformants denotes the integration of at least one copy of the expression cassettes into the chromosome. Hyg^{R} and phytase enzyme activity confirmed the presence of the integrated expression cassettes. Over 800 T. reesei RUT-C30 hyg^{R} transformants were isolated, 240 containing the pTrPh-23 expression cassette and 566 containing the pTrPh-28 expression cassette.

Screening of hyg^{R} transformants and culture-tube expression. Ninety-eight hyg^{R} were analyzed for phytase production. Eight of the 98 produced low levels of phytase activity (Table 14). Transformant TrPh170 was determined microscopically to be the fungal contaminant Penicillum, while the other seven phytase-producing transformants were visually confirmed to be T. reesei RUT-C30. TrPh150 was chosen as a representative transformant because it expressed the highest level of rPhy in its supernatant (Table 14). Phytase activity in the supernatant of TrPh150 grown under shake-flask conditions was similar to the level of phytase activity in the culture-tube experiments (Table 14). Typically, upon scale-up to shake-flask level an increase in protein expression is observed, however this was not the case with TrPh150.

TABLE 14
Phytase activity of T. reesei RUT-C30 (KPF0019-Phy) transformants

| | | | Phytase |
|---|---|---|---|
| Growth | Expression cassette | | activity |
| volume | pTrPh-23 | pTrPh-28 | μmol/min/ml |
| 5 ml | | TrPh002 | 0.038 |
| | | TrPh003 | 0.049 |
| | | TrPh005 | 0.013 |
| | | TrPh150¹ | 0.392 ± 0.05 |
| | TrPh155 | | 0.025 |
| | TrPh170 | | 0.199 |
| | TrPh172 | | 0.002 |
| | TrPh176 | | 0.008 |
| | Control² | | 0.000 |
| 50 ml | TrPh150¹ | | 0.431 ± 0.19 |
| | Control² | | 0.000 |

¹Data presented represent two experiments, all others are one experiment
²T. reesei RUT-C30.

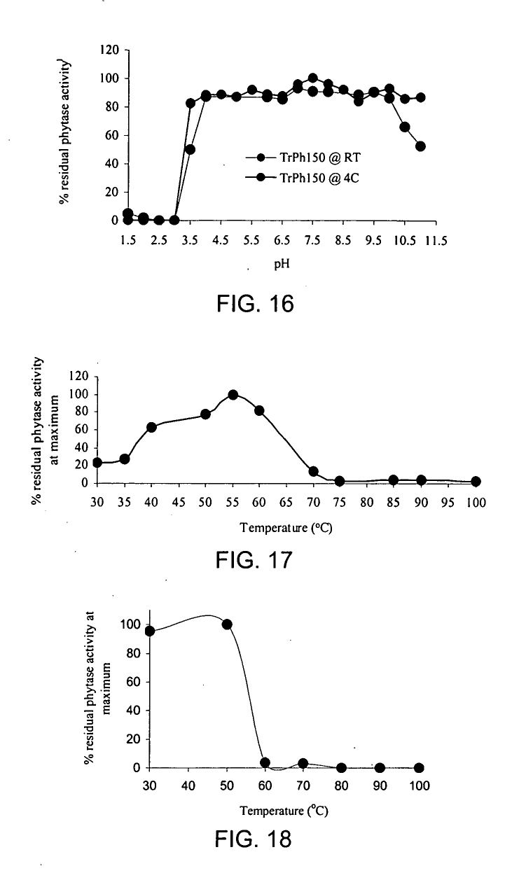

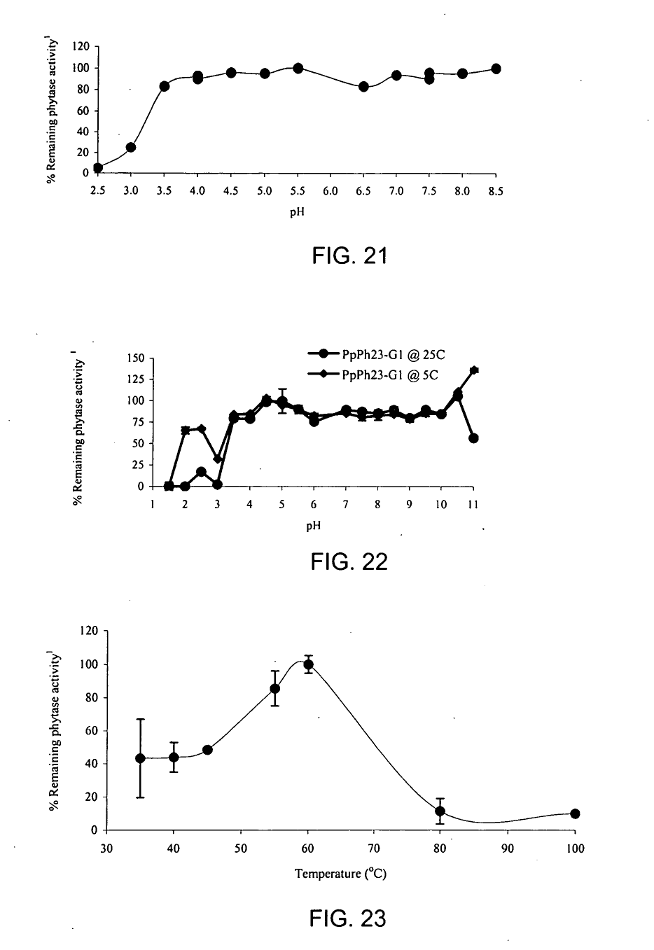

Properties of crude rPhy preparation. The critical to quality parameters of pH stability, pH optimum, temperature stability, and temperature optimum were examined for TrPh150 expressed rPhy. The pH optimum for sodium phytate was 5.5, with 70% of its optimal activity remaining between pH 4.0 and 7.0 (FIG. 15). The enzyme was stable over a large pH range when stored at either ambient or refrigerated temperatures, retaining greater than 50% of its optimal activity after 24 incubation at pHs ranging from 3.5-11.5 (FIG. 16). The temperature optimum for rPhy was determined to be 55° C., which is identical to the temperature optimum of the native enzyme (FIGS. 4 and 21). The enzyme retained greater than 40% of its optimal activity between the temperatures of 37 and 65° C. (FIG. 17). rPhy was stable up to 55° C., but activity dropped rapidly when the enzyme was incubated above this temperature (FIG. 18). Unlike the native enzyme and the P. pastoris rPhy, the T. reesei RUT-C30 rPhy was unable to recover any activity when heated to temperatures above 60° C. then cooled and assayed (FIG. 18). It is unclear as to why T. reesei RUT-C30 rPhy is unable to recover after heat treatment, but it may be a result of posttranslational modification of the enzyme. This may also explain why activity of the enzyme is extremely low, as compared to the native and P. pastoris rPhy. However, pH stability, pH optimum, and temperature optimum were not significantly altered in the T. reesei RUT-C30 rPhy.

Summary. Through an extensive transformation and screening campaign we isolated seven T. reesei RUT-C30 transformants that produced detectable levels of rPhy. These seven rPhy producers represent 0.87% of the total number of stable transformants isolated.

Research has shown that to achieve high-level gene expression in T. reesei foreign genes must be targeted to transcriptionally active sites in the chromosome. The cbhI locus is one of the most transcriptionally active regions and integration at this locus generally yields high-expressing transformants. Integration at the cbhI locus can be accomplished via transformation with foreign genes fused to DNA sequences related to the locus, i.e. the cbhI promoter. This approach has been used many times to create high-level protein producing strains (29, 33, 35, 37). On the other hand, research has also shown that homologous recombination at the cbhI locus does not always result in production of high-expressers (33, 34, 35). In addition, T. reesei rarely homologously recombines DNA, with some estimates showing the level to be as low as 2% (35). Instead, the majority of the time this organism non-homologously integrates foreign DNA at random and transcriptionally dormant sites. This type of integration event would account for the low number of rPhy producing transformants isolated by our screen.

The T. reesei expressed rPhy was found to have very similar biochemical properties as compared to the native KPF0019 phytase enzyme, except with respect to its thermostability. The T. reesei expressed rPhy was unable to recover any of its activity when heated above 60° C. then cooled and assayed, whereas the native enzyme and the P. pastoris rPhy are able to recover 20-50% of their maximum activity under the same experimental conditions. Protein expression levels in T. reesei were also 3-fold lower than that expressed by P. pastoris (discussed in Example 7).

### EXAMPLE 7—EXPRESSION OF THE KPF0019 GENE IN Pichia pastoris

In this Example we describe the use of the methylotrophic yeast Pichia pastoris as a host for the production of recombinant KPF0019 phytase. The KPF0019 phytase gene was fused to the mating factor α1 secretion signal of Saccharomyces cerevisiae and fusion expression was driven by the glyceraldehydes-3-hydrogenase gene promoter. Soluble and active recombinant KPF0019 phytase was secreted into the culture medium. Higher levels of phytase expression were achieved when the cells were cultured by fed-batch fermentation. Recombinant KPF0019 phytase produced by P. pastoris was subjected to N-terminal protein sequence determination and glycosylation analysis. The biochemical properties of the recombinant KPF0019 phytases are described.

Pichia pastoris is a methylotrophic yeast that can grow on methanol as the sole carbon and energy source (38). Because this organism has the ability to produce high-levels of cytosolic or secreted recombinant proteins, it is extensively employed for the industrial-scale production of biologically active proteins. There are many attractive features of this system including a well-defined genetic system, a wide-range of commercially available expression vectors, efficient protein secretion, very low level of endogenous protein secretion, growth to very high cell densities on defined media, and scalable fermentation to the industrial level (38). The inducible alcohol oxidase (AOX1) promoter is widely utilized for the regulated over-expression of foreign genes in P. pastoris, whereas the glyceraldehydes-3-dehydrogenase (GAP) promoter is used for strong, constitutive expression. In general, recombinant, secreted proteins become the majority of the total protein in the P. pastoris culture medium, which greatly facilitates downstream processing. There have been reports of P. pastoris producing 30 g/L intracellular recombinant protein and 18 g/L secreted recombinant protein. This Example describes the genetic engineering and over-expression of the KPF-phy gene in P. pastoris.

A. Materials and Methods

Strains, plasmids, and media. Plasmids and strains used in this study are listed in Table 15. Escherichia coli strain XL1-Blue MRF′ (Stratagene, LaJolla, Calif.) was grown in low salt Luria-Burtani (LB) broth (per liter: Bacto tryptone, 10 g; Bacto yeast extract, 5 g; NaCl, 5 g) or on low salt LB agar (low salt LB broth plus 1.5% Bacto agar) and supplemented with 25 μg/mL of zeocin™ (Invitrogen, Carlsbad, Calif.) when used for propagation of recombinant plasmids. Pichia pastoris strains GS115 and KM71H (Invitrogen) were grown in Yeast Extract Peptone Dextrose Medium (YPD; 2% peptone, 2% dextrose, and 1% Yeast Extract) or YPD agar (YPD broth plus 2% Bacto agar) and supplemented with 100 μg/mL of zeocin™ when used for selection of recombinant plasmid integration events.

TABLE 15
Strains and plasmidsused in Example 7

| Plasmid or | | |
|---|---|---|
| Strain | Relevant genotype | Reference |
| Strains | | |
| Escherichia | Δ (mcrA)183 | Stratagene, La |
| coli | Δ(mcrCB-hsdSMR-mrr)173 | Jolla, CA |
| | endA1supE44 thi-1 | |
| XL1-Blue | recA1 gyrA96 relA1 lac | |
| MRF' | [F′ proAB lacI^{q}Z D M15 Tn10 (Tet^{r})] | |
| Pichia | Mut^{S} His⁺, where Mut^{S} corresponds | Invitrogen, |
| pastoris | to a slow methanol utilization phenotype | Carlsbad, CA |
| KM71H | | |
| Pichia | Mut⁺ His⁻, where Mut⁺ corresponds | Invitrogen, |
| pastoris | to a fast methanol utilization phenotype | Carlsbad, CA |
| GS115 | | |
| G-pKB | P. pastoris strain GS115 (pGAPZ) | Example 7 |
| K-pKB | P. pastoris strain KM71H (pGAPZ) | Example 7 |
| PpPh23-G1 | P. pastoris strain GS115 (pPpPh-23) | Example 7 |
| K23-21 | P. pastoris strain KM71H (pPpPh-23) | Example 7 |
| Plasmids | | |
| pEcPh-1 | KPF-phy::pCR ® 2.1-TOPO ® | Example 2 |
| pGAPZ | P. pastoris expression vector, P_{GAP}, sh ble | Invitrogen, |
| | (zeocin ™ resistance gene) | Carlsbad, CA |
| pPpPh-23 | P. pastoris expression vector, P_{GAP}, sh ble | Example 7 |
| | (zeocin ™ resistance gene), KPF-phy | |

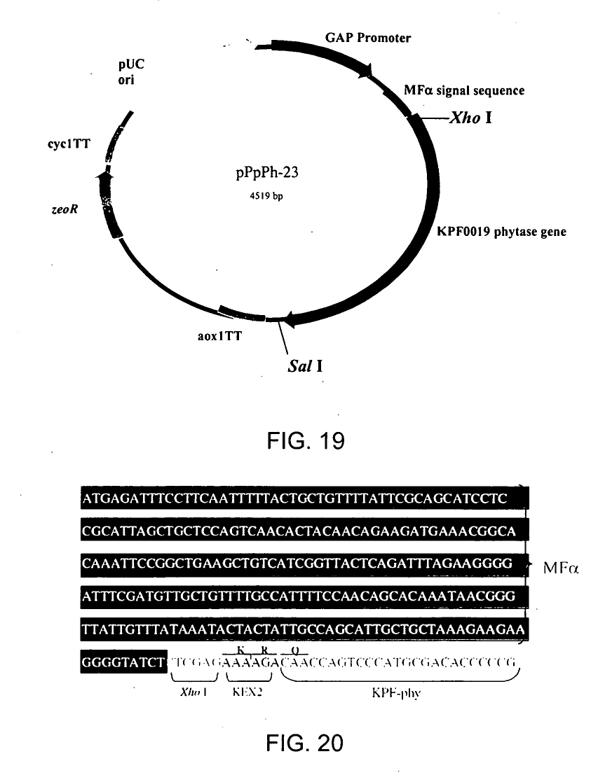

Cloning of the KPF0019 phytase gene into plasmid pGAPZ. In plasmid pPpPh-23 the sequence of the alpha factor secretion signal of Sacchromyces cerevisae is fused in-frame to the 23rd codon of the KPF-phy gene and expression is driven by the constitutive GAP promoter (P_{GAP}) of P. pastoris (FIG. 19). Plasmid pPpPh-23 was created by PCR amplifying a 1446 basepair (bp) region of the native KPF-phy gene using upstream oligonucleotide primer PicF-23 (5′-TCCCTCGAGAAAAGACAACCAGTCCCATGCGAC-3′) in conjunction with downstream oligonucleotide primer PicR-SalI (5′-ACGCGTCGACCTAAGCAAAACACTTGTCCCAAT-3′). The 5′ end of PicF-23 primer contains an artificial Xho I site, a Lys codon, and an Arg codon (together representing the KEX2 cleavage site) followed by nucleotide sequence complementary to the native KPF-phy gene beginning at nucleotide 132 (numbering according the gDNA clone). The 5′ end of PicR-SalI primer contains an artificial Sal I site followed by nucleotide sequence complementary to the 3′ end of the native KPF-phy gene, including the native stop codon. Each 50 μl PCR reaction mixture contained approximately 10 ng pEcPh-1 template DNA, 500 nM of each primer, 200 μM dNTPs, 1×PFU Turbo Buffer (Stratagene) and 2.5 U PFU Turbo Polymerase (Stratagene). The thermocycling program included one cycle at 95° C. (5 min) and 35 cycles of 95° C. (30 s), 60° C. (1 min) and 72° C. (1.5 min) immediately followed by 72° C. (10 min) and an indefinite hold at 4° C. Amplified PCR product was visualized by electrophoresis through a 1% agarose gel containing 0.1 μg/mL ethidium bromide (28, 36). Gel slices containing the expected sized bands were excised and the DNA eluted using the Qiagen Gel Extraction Kit (Qiagen, Valencia, Calif.). PCR product was digested with Sal I and Xho I, visualized, and purified as described above. The Sal I-Xho I digested PCR product was ligated into the Sal I-Xho I sites of plasmid pGAPZ and transformed into E. coli XL1-Blue MRF′. The sequence the KPF-phy gene in pPpPh-23 was confirmed by DNA sequencing performed at the Iowa State University DNA Sequencing and Synthesis Facility (Ames, Iowa) using the dideoxy method via the ABI PRISM Dye Terminator Cycle Sequencing Kit (Applied Biosystems, Foster City, Calif.) and analysis with either the ABI Model 377 Prism DNA Sequencer or the ABI 3100 Genetic Analyzer (Applied Biosystems).

P. pastoris transformation and culture-tube expression of recombinant KPF0019 phytase. Cells of P. pastoris strains KM71H and GS115 were transformed by electroporation with 5 μg of Avr II linearized pPpPh-23 according to the method of Sears et al. (40). Immediately following electroporation cells were plated on YPD agar containing 100 μg/mL zeocin™ (YPDZ₁₀₀) and incubated for 2 days at 30° C. Resultant colonies were re-streaked onto YPDZ₁₀₀ and grown for 2 days at 30° C. to confirm their phenotype. Thirty-two zeocin™-resistant (zeo^{R}) transformants of each P. pastoris strain type were inoculated into 14 mL Falcon tubes containing 1 mL YPD broth and grown overnight at 30° C. and 300 rpm. After growing overnight, biomass was removed by centrifugation and an aliquot of each sample was assayed for phytase activity using the microtiter plate method described in Example 1, with some minor modifications. The most notable modification was that each sample served as its own control. Controls consisted of addition of TCA to each sample prior to phytate addition, followed by incubation at 37° C. for one hour. Based on the results of the phytase activity assay, two transformants were chosen for further study, PpPh23-G1 and K23-21. In the culture-tube experiments PpPh23-G1 and K23-21 were inoculated into glass culture-tubes containing 3 mL of YPD broth and grown for 3 days at 30° C. at 300 rpm. One mL samples were collected daily from each culture-tube for a total of 3 days. The volume in the glass culture-tubes was replaced after each sample draw by addition of 1 mL fresh YPD broth. Each 1 mL growth sample was transferred to a sterile microcentrifuge tube, centrifuged at 14,000 rpm for 1 min (to remove biomass), and the supernatant transferred to a clean, sterile microcentrifuge tube. The remainder of the sample was stored at −20° C. until use. Aliquots of each sample were also analyzed for rPhy production by SDS-PAGE.

Analytical methods. 10% NuPAGE® Novex Bis-Tris [Bis(2-hydroxyethyl) imino-tris (hydroxymethyl) methane-HCl] Pre-Cast Gels (Invitrogen) were used for separating proteins present in spent culture broth supernatant according to manufacture's instructions. Proteins in NuPAGE gels were visualized by staining with GelCode Blue (Pierce Biotechnology, Rockford, Ill.). Glycoprotein staining was performed with the GelCode Glycoprotein Staining Kit (Pierce Biotechnology). Deglycosylation of rPhy was done by treating 5 μl of PpPh23-G1 spent culture broth supernatant with 500 U endoglycosidase H (Endo H) for 1 h at 37° C. according to manufacturer's instructions (New England Biolabs, Beverly, Mass.), except that 0.05 M Na Acetate, pH 5.5 was used instead of 0.05 M Na Citrate, pH 5.5. Elevated Endo H units were utilized to ensure complete deglycosylation of non-denatured rPhy protein. N-terminal amino acid sequencing was performed by electroblotting SDS-PAGE-resolved rPhy proteins onto a polyvinylidene difluroide membrane (BioRad) using a 10 mM CAPS buffer (pH 11) with 10% (v/v) methanol. The protein blot was stained by GelCode Blue. The two potential rPhy bands were then excised from the blot for N-terminal sequencing at the Nucleic Acid-Protein Service Unit at the University of British Columbia.

Biochemical methods. Biochemical analyses were conducted on rPhy present in the spent culture broth of strain PpPh23-G1. The pH profile of rPhy was determined by first adjusting enzyme samples to pHs between 2.5-8.5 using various buffering systems (0.1M formate, pH 2.5-3.5; 0.1M acetate, pH 4.0-5.5; 0.1M Bis-Tris, pH 6.0-7.0; 0.1M Tris-HCl, pH 7.5-8.5). Then five mM phytic acid (at the same pH as the sample) was added and the samples were incubated at 37° C. for 60 min. Following incubation, phytase activity was measured using the microtiter plate method described in Example 1 with minor modifications, as stated above. The temperature profile of rPhy was determined by heating enzyme samples with 5 mM phytic acid at temperatures between 25-100° C. for 60 min followed by measurement of phytase activity. The pH stability profile of rPhy was determined by adjusting the pH of enzyme samples to between pH 3.0 and 8.0 followed by 24 h incubation at 4° C. and 25° C., respectively. After 24 h, samples were adjusted to pH 5.5 and phytase activity was determined. The temperature stability of rPhy was determined by subjecting enzyme samples to various temperatures (between 30-100° C.) for 20 minutes. After heating, samples were cooled on ice and assayed for phytase activity at 37° C.

Expression of rPhy under fermentative conditions. Transformant PpPh23-G1 was chosen to test for rPhy production under fermentative conditions. A 300-mL seed culture of PpPh23-G1 was grown in food-grade YPD medium [1.0% (w/v) FNI 200 yeast extract (Lallemand), 2.0% (w/v) Hy-Soy peptone (Quest International), 2.0% (w/v) dextrose] for 24 h at 30° C., 200 rpm. This culture was used to inoculate a 14-L fermentor (New Brunswick Scientific Co.) containing 8 L of Basal Salt Medium with 40 g·L⁻¹ dextrose, 400 mg/L L-histidine, 0.9 mg/L biotin, and 1×PTM1 trace element solution (39). The fermentor temperature was controlled at 30° C. and dissolved oxygen maintained at 20% via agitation manipulations. The pH was regulated at 5.5 with 100% ammonium hydroxide, which was also used as a nitrogen source. Aeration was maintained at ca. 1 vvm throughout the fermentation. A 5% (w/v) solution of Struktol J673 defoamer (Qemi International) was added as needed to control foaming. Upon depletion of dextrose in the fermentor, a feed containing 50% (w/v) Cerelose (dextrose), 0.7 g/L L-histidine, 2.1 mg/L biotin, and 6×PTM1 trace element solution was initiated at 3 g/L/h dextrose. The feed rate was increased over a period of 25 hr to a maximum of 7 g/L/hr. During feed, dissolved oxygen was maintained at >10% first with agitation manipulations until maximum agitation had been achieved, followed by feed regulations. Cultures were sampled daily to monitor cell density and rPhy production.

B. Results and Discussion

Cloning the KPF0019 phytase gene into pGAPZ and transformation of P. pastoris. The aim of this study was to over-produce soluble and active phytase protein in P. pastoris. P. pastoris has the ability to produce high levels of recombinant protein and secrete them into the surrounding growth media, which greatly simplifies the recovery process and reduces the cost of manufacturing a final product. A 5′ truncated version of the KPF-phy gene was inserted into the Xho I-Sal I sites of the P. pastoris constitutive expression vector pGAPZ, forming pPpPh-23 (FIG. 19). This plasmid contains a N-terminal translational fusion of the alpha factor secretion signal (MFα) (plus the Pro-region), a KEX2 protease recognition sequence ending with Lys-Arg, and the KPF-phy gene sequence starting at codon 23 (bp 132) (FIG. 20). The constitutive glyceraldehyde-3-phosphate dehydrogenase promoter (P_{GAP}) drives expression of the fusion in P. pastoris. In the endoplasmic reticulum the first 19 amino acids of the MFα peptide are cleaved by signal peptidase and in the Golgi the KEX2 protease cleaves the MFα Pro-region-phytase fusion at the Pro-region after the Lys-Arg dipepetide (FIG. 20). This cleavage results in a mature, recombinant phytase protein beginning with glutamine (FIG. 20). In order to increase homologous recombination efficiency, plasmid pPpPh-23 was linearized with Avr II prior to electroporation into P. pastoris strains GS115 and KM71H. Since plasmid pPpPh-23 lacks a yeast origin of replication, it cannot autonomously replicate in P. pastoris. Therefore, the recovery of zeo^{R} transformants denotes the integration of at least one copy of the linearized plasmid into the chromosome of P. pastoris and homologous recombination occurs within the upstream 5′ sequence of the GAP promoter region of the P. pastoris chromosome. Zeo^{R} and phytase enzyme activity confirmed the presence of the integrated plasmid.

Screening of zeo^{R} transformants and culture-tube expression. Two genetically distinct strains of P. pastoris (GS115 and KM71H) were transformed with the pPpPh-23 and 32 zeo^{R} colonies of each strain type were examined for phytase activity. Negative controls consisted of the two P. pastoris strains transformed with pGAPZ, which contains the does not contain the KPF0019-Phy gene (Table 16).

TABLE 16
Phytase activity in culture broths of P. pastoris
pPpPh-23 transformants.

| | | | Phytase |
|---|---|---|---|
| | Phytase activity² | | activity² |
| Transformant¹ | (μmol/min/ml) | Transformant³ | (μmol/min/ml) |
| K23-1 | 0.682 | G23-1 | 1.107 |
| K23-2 | 0.689 | G23-2 | 0.974 |
| K23-3 | 0.749 | G23-3 | 0.759 |
| K23-4 | 0.858 | G23-4 | 0.918 |
| K23-5 | 0.756 | G23-5 | 0.859 |
| K23-6 | 0.655 | G23-6 | 1.027 |
| K23-7 | 0.706 | G23-7 | 0.853 |
| K23-8 | 0.569 | G23-8 | 1.001 |
| K23-9 | 0.495 | G23-9 | 1.076 |
| K23-10 | 0.728 | G23-10 | 0.913 |
| K23-11 | 0.682 | G23-11 | 1.012 |
| K23-12 | 0.689 | G23-12 | 0.848 |
| K23-13 | 0.664 | G23-13 | 0.834 |
| K23-14 | 0.611 | G23-14 | 0.843 |
| K23-15 | 0.677 | G23-15 | 0.878 |
| K23-16 | 1.510 | G23-16 | 0.961 |
| K23-17 | 1.474 | G23-17 | 1.149 |
| K23-18 | 1.481 | G23-18 | 1.023 |
| K23-19 | 1.465 | G23-19 | 0.968 |
| K23-20 | 1.157 | G23-20 | 0.986 |
| K23-21 | 1.571 | G23-21 | 0.843 |
| K23-22 | 1.424 | G23-22 | 0.909 |
| K23-23 | 1.402 | G23-23 | 0.869 |
| K23-24 | 1.647 | G23-24 | 1.076 |
| K23-25 | 1.528 | G23-25 | 1.081 |
| K23-26 | 1.635 | G23-26 | 1.125 |
| K23-27 | 1.532 | G23-27 | 1.023 |
| K23-28 | 1.479 | G23-28 | 1.236 |
| K23-29 | 1.541 | G23-29 | 1.047 |
| K23-30 | 1.548 | G23-30 | 1.234 |
| K23-31 | 0.958 | G23-31 | 1.253 |
| K23-32 | 0.973 | G23-32 | 1.041 |
| K-pKB⁴ | 0 | G-pKB⁵ | 0 |

¹Generated by transformation of P. pastoris strain KM71H with pPpPh-23.
²Data are the result of one experiment.
³Generated by transformation of P. pastoris strain GS115 with pPpPh-23.
⁴Negative control generated by transformation of P. pastoris strain KM71H with pGAPZ.
⁵Negative control generated by transformation of P. pastoris strain GS115 with pGAPZ.

All P. pastoris strains transformed with pPpPh-23 displayed low levels of phytase activity in their culture broth supernatants and no activity was present in the negative controls (Table 16), indicating that rPhy was successfully expressed and secreted. Phytase activity expressed by transformants G23-30 (renamed PpPh23-G1) and K23-21 were representative of all zeo^{R} transformants isolated, therefore these two transformants were chosen for further study. Table 17 shows the results of a 3-day culture-tube expression study. Similar levels of phytase activity were observed in the culture broth supernatants of transformants PpPh23-G1 and K23-21 on all three days. Longer growth times did not correlate with increased rPhy yields.

TABLE 17
Phytase assay of spent culture broths of KPF-phy
P. pastoris transformants.¹

Phytase activity
(μmol/min/ml)²

| Transformant | Day 1 | Day 2 | Day 3 |
|---|---|---|---|
| PpPh23-G1 | 1.7284 | 1.487 | 1.523 |
| G-pKB (negative control) | ND³ | 0.020 | 0.076 |
| K23-21 | 1.521 | 1.224 | 1.105 |
| K-pKB (negative control) | 0.016 | 0.0 | 0.0 |

¹Data presented are from one experiment. The experiment has been repeated twice with the same results.
²Phytase activity was determined using the microtiter plate method of Example 1.
³ND, not determined.

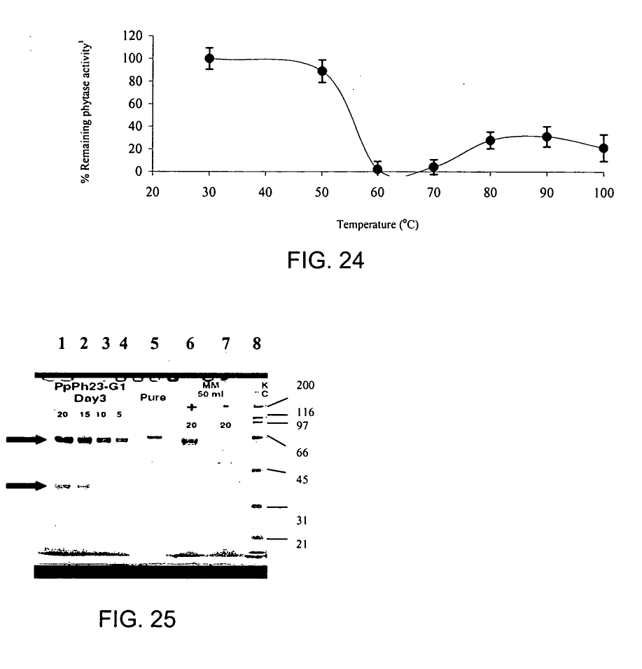

Properties of crude rPhy preparation. The critical to quality parameters of pH stability, pH optimum, temperature stability, and temperature optimum were examined for P. pastoris expressed rPhy. The pH optimum for sodium phytate was 5.5, with 80% of its optimal activity remaining between pH 3.5-8.5 (FIG. 21). The enzyme was stable over a large pH range when stored at either ambient or refrigerated temperatures, retaining greater than 50% of its optimal activity after 24 incubation at pHs ranging from 3.5-11 (FIG. 22). The temperature optimum for rPhy was determined to be 60° C. (FIG. 23). The enzyme retained greater than 40% of its optimal activity between the temperatures of 35 and 70° C. (FIG. 23). rPhy was stable up to 55° C., however, activity dropped rapidly when the enzyme was incubated above this temperature (FIG. 24). When incubation temperatures exceeded 70° C. rPhy was able to recover between 20 and 40% of its optimal activity (FIG. 24). This may be due to its ability to refold after complete denaturation at elevated temperatures, whereas at lower temperatures (60° C.) denaturation is incomplete and thus interferes with proper refolding. These biochemical properties are nearly identical to the biochemical properties of the crude extract of KPF0019 phytase, indicating that P. pastoris is producing a bioactive molecule essentially identical to the native enzyme.

Production of rPhy by strain PpPh23-G1. Initially we thought that phytase transformants PpPh23-G1 and K23-21 had low rPhy production levels because their supernatants had low-levels of phytase activity (Tables 20 and 21). To determine if this was the case, supernatant from the transformants' culture broths were also examined for protein production by SDS-PAGE. Both transformants appear to produce significant levels of rPhy (FIG. 25) (K23-21 data not shown). Surprisingly, each transformant's supernatant contained two predominant bands, one of higher molecular weight (MW) (FIG. 25, top arrow) and one of lower MW (FIG. 25, bottom arrow). These two protein bands are not present in the negative control lane (FIG. 25, lane 7), indicating they are related to rPhy expression. The higher MW band is in the acceptable MW range for rPhy (FIG. 25, top arrow). In FIG. 25, the lanes marked MM-50 mL represent spent culture broth supernatants from 50 mL overnight YPD broth shake-flask cultures of PpPh23-G1 (marked as +) and G-pKB (marked as −). The shake-flask culture of PpPh23-G1 produced a similar level of rPhy as compared the amount produced in the culture-tube experiment (FIG. 25, lanes 1-4 versus lane 6). Again, the predominant band just below the 66 kDa molecular weight marker is present only in the supernatant of strain PpPh23-G1, which contains an integrated copy of the KPF-phy gene and not is not found in strain G-pKB, which does not contain the KPF-phy gene (FIG. 25, lane 6 versus lane 7). This provides strong evidence that this protein band is rPhy. N-terminal amino acid sequence analysis provided additional evidence that this band is rPhy. The N-terminal sequencing results were ambiguous and difficult to interpret, however the sequence matched the engineered N-terminus of rPhy (data not shown). The result suggests the KEX2 protease correctly processed the MFα secretion signal peptide-Pro region. The lower MW N-terminal sequencing results were also ambiguous and noisy. However, none of the possible combinations of potential amino acids at each predicted position match any sequence of amino acids in rPhy, showing that this band is not a proteolytic product of rPhy (data not shown).

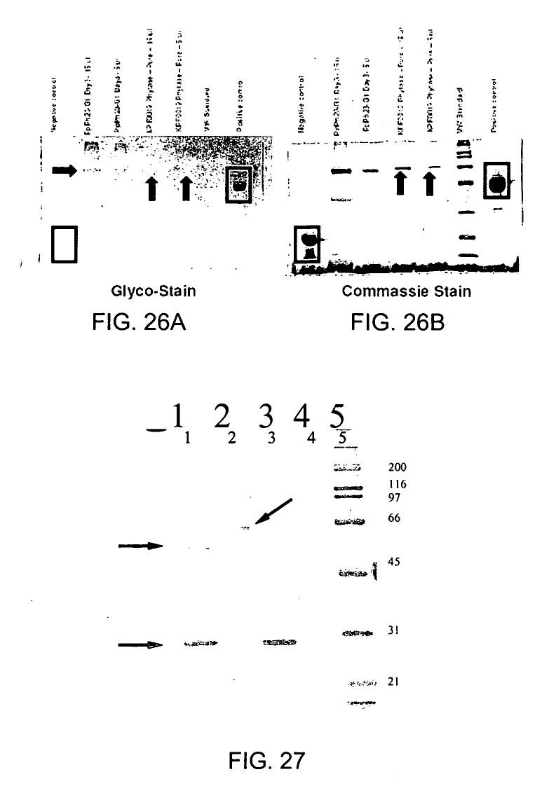

Analysis of rPhy glycosylation. The rPhy produced by strain PpPh23-G1 has a higher MW than expected as compared to the calculated MW of 52,776 daltons based on the deduced amino acid sequence of the KPF-phy gene. Glycosylation of rPhy could account for the observed difference in MW. A glycoprotein-stain that specifically binds to the oxidized sugar-moieties present in glycoproteins was used to determine if the putative rPhy is glycosylated. Unglycosylated proteins present in the SDS-PAGE gel will not be stained using this method. In FIG. 26A is the glycoprotein-stained SDS-PAGE gel and in FIG. 26B is the same gel stained with GelCode Blue (after glycoprotein staining). The rPhy protein was stained by the Glycoprotein Staining Kit indicating that it is N-glycosylated (FIG. 26A). Positive and negative controls were also electrophoresed through the same SDS-PAGE gel to ensure validity of the experimental result. As shown in FIG. 26A, the positive control reacts with the glycoprotein stain whereas the negative control does not (upper right boxes vs. lower left boxes).

The type and degree of glycosylation cannot be determined using this staining method; therefore, we used Endoglycosidase H (Endo H) to treat rPhy to investigate the glycosylation further. Endo H is a glycosidase, which cleaves the chitobiose core of high mannose and some hybrid oligosaccharides from N-linked glycoproteins. Endo H treated rPhy was also examined for phytase activity to determine if glycosylation was affecting phytase activity. Deglycosylation of rPhy had no effect on the phytase activity of rPhy (data not shown). SDS-PAGE analysis showed a series (3-4) of protein bands of lower MW appearing in the Endo H treated rPhy lane as compared to untreated rPhy control (FIG. 27, lanes 1 and 2), indicating that rPhy is N-glycosylated. The most predominant of the deglycosylated bands has an apparent MW of 55 kDa, which is very close to the predicted MW of rPhy.

Production of rPhy by fermentation. Although heterologous proteins can be expressed well in P. pastoris shake-flask cultures, expression levels are typically low when compared to fermentative cultures. One reason is that only in the controlled environment of a fermentor is it possible to grow this organism to high cell density (OD₆₀₀ unit 500) (39). Especially for secreted proteins, the concentration of product in the culture medium is roughly proportional to the cell density in the fermentor. Because of these reasons, we decided to examine rPhy production by clone PpPh23-G1 under fermentative conditions. The fermentation process is run in fed-batch mode. Dextrose serves as the sole carbon source and is maintained at a limited (>0.5%) concentration in the culture broth once initial dextrose is consumed. Cultures were sampled ca. every 24 hr and were fractioned into biomass and supernatants for SDS-PAGE analysis and phytase activity assay. SDS-PAGE confirmed the accumulation of rPhy as the major protein secreted by PpPh23-G1 (FIG. 28, arrow). The analysis also showed that fermentation of PpPh23-G1 increased rPhy production approximately 3- to 5-fold over culture-tube production (FIG. 28, lanes 2 and 3 versus lane 5). At 24 hours post-inoculation (HPI) phytase activity resembled that of previous culture-tube and shake-flask experiments (Table 18). However, by 70 HPI phytase activity present in the supernatant was 15-fold higher than in a typical culture-tube supernatant (Table 18). Phytase production essentially ceased after 70 HPI, a phenomenon that has been observed with other P. pastoris strains run under fed-batch conditions.

TABLE 18
Phytase activity from PpPh23-G1 under fermentative conditions

| HPI | Phytase, U/ml |
|---|---|
| 0 | 0 |
| 24 | 1.3 |
| 47 | 10.5 |
| 70 | 23.1 |
| 93 | 22.4 |
| 120 | 23.3 |
| 140 | 19.3 |

Summary. We have shown that under culture-tube, shake-flask, and fermentative growth conditions rPhy can be expressed at relatively high levels in P. pastoris. Overall, a 15-fold increase in phytase activity was observed in the 10-L fermentation of strain PpPh23-G1 as compared to the same strain grown in a culture-tube or shake-flask. Approximately 3- to 5-fold of this increase was due to an increase in protein production. The remaining 10- to 12-fold increase in expression seems to be due to the fermentation media, the growth conditions, or more likely a combination of both. It is important to note that the fermentation conditions were an initial effort to grow the strain and are not optimized. Therefore it is likely expression can be increased even further through fermentation optimization.

The biochemical characteristics of rPhy expressed by P. pastoris and crude extract of KPF0019 phytase are nearly identical. The rPhy was less stable at pH 3 as compared to the KPF0019 phytase, but more stable between pH 4-10. rPhy showed a 5° C. shift in its temperature optimum as compared to the KPF0019 phytase, which could be due to glycosylation of rPhy. Both enzymes exhibited similar temperature stability profiles, with optimal activity falling to zero when exposed to temperatures between 60-70 C for 30 minutes. Each enzyme was able to recover 20-50% of its maximum activity when heated between 80-100 C. There could be several explanations for this phenomenon. A likely scenario is that at lower temperatures (60-70° C.) the KPF0019 and rPhy enzymes only partially denature, therefore when the sample is cooled they cannot refold into active forms. Conversely, at elevated temperatures (80-100° C.) the enzymes are completely denatured and upon cooling a small percentage are able to properly refold into a bioactive form. The ability to refold after treatment at high temperatures is a desirable quality in enzymes that are used in pelleted animal feed.

The post-translational modification of proteins by the addition of sugar residues can significantly affect protein stability, conformation, and functional activity. Glycosylation also plays an important role in cell-to-cell and intracellular protein targeting. These factors can have important effects on the commercial development of recombinant products. Although the absolute nature of the post-translational modification is unclear, experimental evidence shows that rPhy produced by Pichia is N-glycosylated. Endo H treatment was able to deglycosylate rPhy to an apparent MW of 55 kDa, in effect accounting for the entire shift in molecular weight observed with the rPhy protein. Glycosylation is not responsible for the elevated MW the purified KPF0019 phytase. Since KPF0019 is likely a Neurospora species and these organisms are known to glycosylate their proteins, it is surprising that the purified KPF0019 phytase enzyme was not found to be glycosylated. Another potential issue that arose from the SDS-PAGE results was the presence of a lower molecular weight band (between 31 and 45 kDa). This protein band was not observed in the negative control indicating its presence might be related to expression of rPhy. One hypothesis was that the peptide is a proteolytic product of rPhy. However, N-terminal sequencing showed that although the approximately 66 kDa was rPhy, the lower molecular weight band did not contain sequences related to rPhy (data not shown). The protein is likely native to Pichia and present due to increased cell lysis in rPhy producing cells.

Most organisms display non-random patterns of synonymous codon usage (41, 42). When the DNA sequence of the KPF-phy gene was compared to the codon usage preferences of P. pastoris we found a pronounced difference in usage patterns. This dramatic difference suggested that P. pastoris would not efficiently translate the native KPF-phy gene resulting in poor rPhy production. This appeared to be the situation according to the results of the phytase activity assays performed on pPpPh-23 P. pastoris transformants. Very low phytase activity was measured in the supernatants of transformants PpPh23-G1 and K23-21 indirectly suggesting a low level of rPhy production. However, the SDS-PAGE results reported here demonstrate this is not the case. The native KPF-phy gene when expressed in P. pastoris is efficiently translated despite the pronounced difference in codon usage as evidenced by the appearance of a predominant protein in the transformants that is not found in the negative control supernatant. N-terminal sequencing confirmed the protein is rPhy. Based on this finding and literature references showing significant increases in heterologous protein expression via codon optimization, complete codon optimization of KPF-phy towards P. pastoris bias should result in an increase in what already appears to be a respectable protein production level (41, 42).

### EXAMPLE 8—SYNTHESIS AND EXPRESSION OF CODON-OPTIMIZED KPF0019 PHYTASE GENE IN Pichia pastoris

In this Example we describe the synthesis of a codon-optimized version of this gene based on Pichia pastoris codon usage. As a result of optimization, sixty-seven percent of the native KPF0019 phytase gene codons were altered. The codon-optimized phytase gene was fused to the mating factor α secretion signal of Saccharomyces cerevisiae and the glyceraldehydes-3-hydrogenase gene promoter drove fusion expression. Soluble and active recombinant phytase was secreted into the culture medium. Higher levels of phytase expression were achieved when the cells were cultured by fed-batch fermentation.

Yeasts offer certain advantages over other organisms, since they are eukaryotes; therefore their intracellular environment is likely to be more suitable for the correct folding of other eukaryotic proteins, like rPhy. They also have the ability to glycosylate, which can be important for stability, solubility, and biological activity. Lastly, they can secrete proteins, which facilitates the separation of the desired recombinant products from cellular constituents. P. pastoris has the ability to produce high-levels of cytosolic or secreted recombinant proteins and it is extensively employed by both academic and commercial organizations for the industrial-scale production of biologically active proteins. There are many attractive features of this system including a well-defined genetic system, a wide-range of commercially available expression vectors, efficient protein secretion, very low level of endogenous protein secretion, growth to very high cell densities on defined media, and scalable fermentation to the industrial level (38). There have been reports of P. pastoris producing 30 g/L intracellular recombinant protein and 18 g/L secreted recombinant protein. The inducible alcohol oxidase (AOX1) promoter is widely utilized for the regulated over-expression of foreign genes in P. pastoris, whereas the glyceraldehydes-3-dehydrogenase (GAP) promoter is used for strong, constitutive expression. In general, recombinant, secreted proteins become the majority of the total protein in the P. pastoris culture medium, which greatly facilitates downstream processing.

Most organisms display non-random patterns of synonymous codon usage and show a general bias towards a subset of the 61 possible sense codons (41, 42). Studies have shown that different patterns of codon bias between a transgene and an expression host will have a significant impact on the level of recombinant protein produced. The complete optimization of coding regions towards the codon bias of the host cell can lead to a dramatic increase in protein production. Even the removal of only particularly rare codons throughout a gene has been shown to have a significant impact on heterologous protein production. Yao et al. reported a 37-fold increase in the concentration of phytase expressed by P. pastoris when just the Arg codons of the A.s niger phyA gene were modified (43).

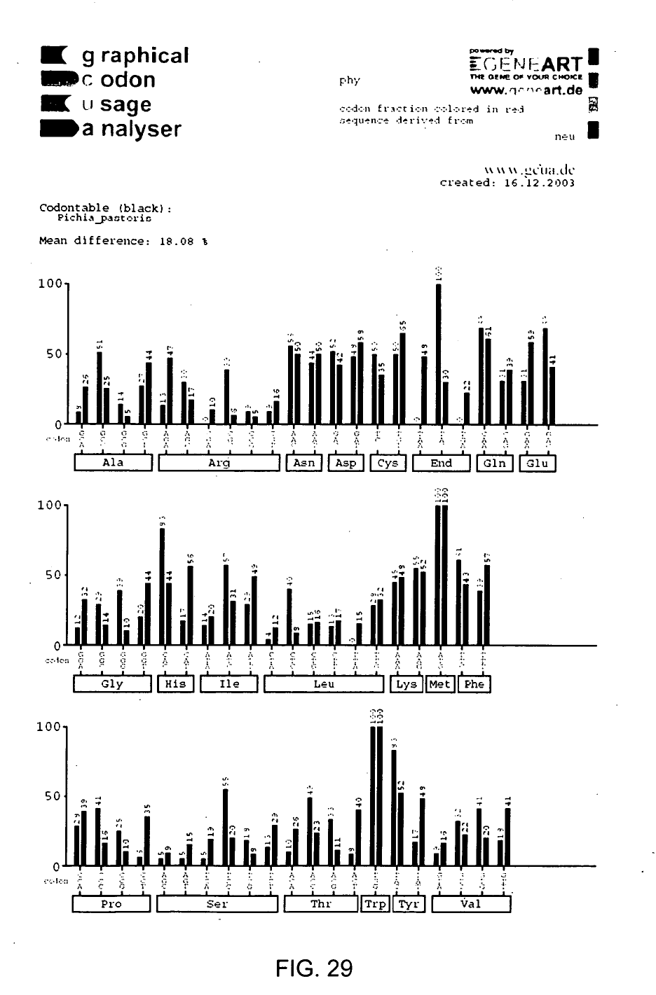

When the DNA sequence of the KPF-phy gene was compared to the codon usage preferences of P. pastoris a pronounced difference in usage patterns was found (FIG. 29). This dramatic difference suggested that P. pastoris would not efficiently translate the native KPF-phy gene resulting in poor rPhy production. This appeared to be the situation according to the results of the phytase activity assays performed on P. pastoris (KPF-phy) transformants (43). Very low phytase activity was measured in the supernatants of transformants indirectly suggesting a low level of rPhy production. However, SDS-PAGE results demonstrated this was not the case. The native KPF-phy gene when expressed in P. pastoris was efficiently translated despite the pronounced difference in codon usage as evidenced by the appearance of a predominant protein in the transformants that was not found in the negative control supernatant. Even though good protein production levels were observed, they were not high enough for commercial-level production. Therefore, to further increase rPhy production by P. pastoris KPF-phy was codon-optimized. In theory, complete codon optimization of KPF-phy towards P. pastoris bias should result in an increase in what already appears to be a respectable protein production level (41, 42). The most straightforward way to generate a desired DNA sequence is simply to synthesis it. This Example describes the in vitro synthesis, cloning, and expression of the codon-optimized KPF-phy (phy^{CO}) gene in P. pastoris.

A. Materials and Methods

Strains, plasmids, and media. Plasmids and strains used in this study are listed in Table 19. Escherichia coli strain XL1-Blue MRF′ (Stratagene, LaJolla, Calif.) was grown in low salt Luria-Burtani (LB) broth (per liter: Bacto tryptone, 10 g; Bacto yeast extract, 5 g; NaCl, 5 g) or on low salt LB agar (low salt LB broth plus 1.5% Bacto agar) and supplemented with 25 μg/mL of zeocin™ (Invitrogen, Carlsbad, Calif.) when used for propagation of recombinant plasmids. Pichia pastoris strain KM71H (Invitrogen) was grown in Yeast Extract Peptone Dextrose Medium (YPD; 2% peptone, 2% dextrose, and 1% Yeast Extract) or on YPD agar (YPD broth plus 2% Bacto agar) and supplemented with either 100 or 250 μg/mL of zeocin™ when used for selection of recombinant plasmid integration events.

TABLE 19
Strains and plasmids used in Example 8

| Plasmid or | | |
|---|---|---|
| Strain | Relevant genotype | Reference |
| Strains | | |
| Escherichia coli | Δ(mcrA)183 Δ(mcrCB-hsdSMR-mrr)173 endA1 | Stratagene, La |
| | supE44 thi-1 | Jolla, CA |
| XL1-Blue | recA1 gyrA96 relA1 lac [F′ proAB lacI^{q}Z D | |
| MRF' | M15 Tn10 (Tet^{r})] | |
| Pichia | Mut^{S} His⁺, where Mut^{S} corresponds to a slow | Invitrogen, |
| pastoris | methanol utilization phenotype | Carlsbad, CA |
| KM71H | | |
| Pichia | Mut⁺ His⁻, where Mut⁺ corresponds to a fast | Invitrogen, |
| pastoris | methanol utilization phenotype | Carlsbad, CA |
| GS115 | | |
| G-pKB | P. pastoris strain GS115 (pGAPZ) | Example 7 |
| PpPh23-G1 | P. pastoris strain GS115 (pPpPh-23); integrated | Example 7 |
| | native KPF-phy gene | |
| PpPh-21co-48 | P. pastoris KM71H (pPpPh-21co), integrated | Example 8 |
| | phy^{CO} gene, transformant number 48, isolated on | |
| | YPDZ₂₅₀ | |
| PpPh-21co-69 | P. pastoris KM71H (pPpPh-21co), integrated | Example 8 |
| | phy^{CO} gene, transformant number 69, isolated on | |
| | YPDZ₂₅₀ | |
| Plasmids | | |
| pGAPZ | P. pastoris expression vector, P_{GAP}, sh ble | Invitrogen, |
| | (zeocin ™ resistance gene) | Carlsbad, CA |
| pPpPh-21co | P. pastoris expression vector, P_{GAP}, sh ble | Example 8 |
| | (zeocin ™ resistance gene), phy^{CO} | |

Design, synthesis, and cloning of the phy^{CO} gene. The synthetic phy^{CO} gene was designed using the DNAWorks Web Site (molbio.info.nih.gov/dnaworks), the deduced amino acid sequence of the KPF-phy gene, and a P. pastoris codon usage table (Codon Usage Database) (4). The deduced amino acid sequence of KPF-phy gene and the P. pastoris codon usage table were entered into the DNAWorks computer program and the output was the sequence of the synthetic phy^{CO} gene sequence with codons optimized for expression in P. pastoris. The output also included a series of overlapping oligonucleotide primer sequences that span the entire phy^{CO} gene sequence. The oligonucleotides are characterized by highly homogeneous melting temperatures and a minimized tendency for hairpin formation, as well as the absence of any Xho I or Sal I restriction endonuclease recognition sequences except at the 5′ and 3′ ends, respectively. The program determined that 60 complementary, overlapping oligonucleotides would need to be synthesized to create the synthetic, codon-optimized phy^{CO} gene. The 5′ end of the F1 primer contains an artificial Xho I site, a Lys codon, and an Arg codon (together representing the KEX2 cleavage site) followed by nucleotide sequence complementary to the synthetic phy^{CO} gene beginning at nucleotide 127 (numbering according the gDNA clone) (Table 20). The 5′ end of the R1 primer contains an artificial Sal I site followed by nucleotide sequence complementary to the 3′ end of the phy^{CO} gene, including the optimized stop codon (Table 21). Synthetic phy^{CO} gene assembly was accomplished through a three-step PCR protocol. Each of the 60 overlapping oligonucleotides (F1-F30 and R1-R30) (Tables 24 and 25) (Qiagen, Valencia, Calif.) were dissolved in sterile dH₂O to a final concentration of 100 μM. Step-one consisted of assembly of the phy^{CO} gene into five fragments, each approximately 300 basepairs (bp) in length. Five oligonucleotide primer mixtures representing the five fragments, were prepared by combining 10 μl of each primer (12 primers per mix, 6 sense, 6 antisense) (final concentration of each primer of 8.3 μM). The primer mixtures were as follows: mixture 1, F1-F6 and R25-R30; mixture 2, F7-F12 and R19-R24; mixture 3, F13-F18 and R13-R18; mixture 4, F19-F24 and R7-R12; and mixture 5, F25-F30 and R1-R6 (Tables 24 and 25). Each 100 μl PCR reaction mixture (1-5) contained 1 μM of each of the 12 primers (12 μl of each primer mixture 1-5, respectively), 250 μM dNTPs, 1×PFU Turbo Buffer (Stratagene) and 5 U PFU Turbo Polymerase (Stratagene). The thermocycling program included one cycle of 94° C. (2 min), 53° C. (2 min), and 72° C. (10 min) followed by 40 cycles of 94° C. (30 s), 53° C. (1 min) and 72° C. (20 sec +3 sec/cycle). A final extension of 72° C. (10 min) was followed by an indefinite hold at 4° C. Amplified PCR product was visualized by electrophoresis through a 1% agarose gel containing 0.1 μg/mL ethidium bromide (11, 28). Gel slices containing the expected sized bands were excised and the DNA eluted using the Qiagen Gel Extraction Kit (Qiagen). The 5 individual DNA fragments were then re-amplified using the end flanking primers for each fragment, respectively. The thermocycling program included one cycle of 94° C. (2 min) followed by 40 cycles of 94° C. (30 s), 60° C. (1 min) and 72° C. (45 sec). A final extension of 72° C. (10 min) was followed by an indefinite hold at 4° C. PCR products were visualized and purified as stated above. The DNA sequence each of the five fragments was confirmed by DNA sequencing performed at the Iowa State University DNA Sequencing and Synthesis Facility (Ames, Iowa) using the dideoxy method via the ABI PRISM Dye Terminator Cycle Sequencing Kit (Applied Biosystems, Foster City, Calif.) and analysis with either the ABI Model 377 Prism DNA Sequencer or the ABI 3100 Genetic Analyzer (Applied Biosystems).

TABLE 20

Oligonucleotide primers used to synthesize
the phy^{CO} gene

| | |
|---|---|
| Primer | Sense strand primers (5′ to 3′) |

| | |
|---|---|
| F1 | TCCCTCGAGAAGAGATCTCCAAGTTAGGTTATCAATGTGACCAA |
| | CAACCAGTTCCATGTGATACTCCAG |
| F2 | AAAACTACTCATACTTGGGGACAATACTCACCATTCTTCTCTGT |
| | TCCATC |
| F3 | TGAGATTTCACCTTCAGTTCCATCTGGATGTAGGTTAACTTTTG |
| | CACA |
| F4 | AGTTTTATCTAGGCATGGAGCTAGATTCCCTACTGCTGGAAAAG |
| | C |
| F5 | TGCTGCTATATCTGCTGTTTTAACAAAGATTAAGACATCTGCTA |
| | CATGGTAC |
| F6 | GCACCAGACTTCGAGTTCATTAAAGATTACAACTATGTTTTGGG |
| | TGTTG |
| F7 | ACCATTTAACAGCTTTTGGTGAACAAGAAATGGTCAACTCAGGA |
| | ATAAAGT |
| F8 | TTTACCAGAGGTATGCTTCATTGTTGAGAGACTACACAGATCCT |
| | GAATC |
| F9 | ATTGCCTTTCGTTAGAGCATCAGGTCAAGAAAGAGTCATTGCAT |
| | C |
| F10 | TGCAAAGAACTTCACTACTGGTTTCTACTCTGCTTTGTTGGCTG |
| | A |
| F11 | CAAAAATCCTCCACCTTCTTCTTTGCCATTGCCTAGACAAGAGA |
| | TGG |
| F12 | TCATTATATCAGAGTCTCCAACAGCAAACAATACAATGCACCAC |
| | GG |
| F13 | TTTGTGTAGAGCTTTTGAAGATTCAACAACTGGAGATTCTGTTC |
| | AGGC |
| F14 | TACTTTCATTGCTGCTAATTTTCCTCCTATTACTGCAAGGTTGA |
| | ACGC |
| F15 | TCAGGGTTTCAAAGGAGTTGAATTATCTGATACAGACGTTTTGT |
| | CATTGATG |
| F16 | GATTTGTGCCCATTTGACACAGTTGCATACCCTCCATCTTCATT |
| F17 | GACTACTTTATCATCACCTTCAAGAGGTTCAAAGTTGTTATCTC |
| | CATTCTGCT |
| F18 | CTTTGTTCACAGCACAAGACTTCACTGTTTACGACTACTTGCAA |
| | TCTT |
| F19 | CTTTGTTCACAGCACAAGACTTCACAGGAAACTTCTTGGGAGCT |
| | ACACAA |
| F20 | GGAGTCGGATACGTTAATGAGTTGTTAGCTAGGTTAACTAGATC |
| | ACCAG |
| F21 | TCGTTGACAACACAACTACTAATTC AACTTTGGATGGAAACGA |
| | GGAAA |
| F22 | CATTTCCATTGACAAAGAACAGAACTGTCTTTGCAGATTTCTCA |
| | CATGAC |
| F23 | AATGACATGATGGGAATATTAACTGCTTTGAGATTGTTCGAAAC |
| | TGTCGA |
| F24 | AGGTATGGATAACACAACAATTCCAAAAGGATACGGATCTACAG |
| | GAGAC |
| F25 | GAACCTGGATTGAAGGAAAGAGAGGGTGTTTTTAAGGTTGGATG |
| | G |
| F26 | GCAGTCCCTTTTGCTGGTAGGGTTTACTTTGAGAAAATGGTTTG |
| | T |
| F27 | GATGGAGACGGAGATGGTGAAATTGATCAAGGTGAGGAGGAG |
| F28 | CAAGAGTTGGTCAGAATTTTGGTCAATGACAGAGTCGTCAAGTT |
| | GAA |
| F29 | CGGATGTGAGGCTGATGAGTTAGGTAGATGCAAATTGGGAAAGT |
| | T |
| F30 | TGTCGAGTCAATGGAATTTGCTAGAAGGGGTGGTGATTGGG |

TABLE 21

Oligonucieotide primers used to synthesize
the phy^{CO} gene

| | |
|---|---|
| Primer | Antisense strand primers (5′ to 3′) |

| | |
|---|---|
| R1 | ACGCGTCGACTTAAGCGAAACACTTGTCCCAATCACCACCCCTT |
| | C |
| R2 | TAGCAAATTCCATTGACTCGACAAACTTTCCCAATTTGCATCTA |
| | CCTAA |
| R3 | CTCATCAGCCTCACATCCGTTCAACTTGACGACTCTGTCATT |
| R4 | GACCAAAATTCTGACCAACTCTTGCTCCTCCTCACCTTGATCAA |
| | TTT |
| R5 | CACCATCTCCGTCTCCATCACAAACCATTTTCTCAAAGTAAACC |
| | C |
| R6 | TACCAGCAAAAGGGACTGCCCATCCAACCTTAAAAACACCC |
| R7 | TCTCTTTCCTTCAATCCAGGTTCGTCTCCTGTAGATCCGTATCC |
| | T |
| R8 | TTTGGAATTGTTGTGTTATCCATACCTTCGACAGTTTCGAACAA |
| | TCTCAA |
| R9 | AGCAGTTAATATTCCCATCATGTCATTGTCATGTGAGAAATCTG |
| | CAAAGAC |
| R10 | AGTTCTGTTCTTTGTCAATGGAAATGTTTCCTCGTTTCCATCCA |
| | AAGT |
| R11 | TGAATTAGTAGTTGTGTTGTCAACGACTGGTGATCTAGTTAACC |
| | TAGCTAAC |
| R12 | AACTCATTAACGTATCCGACTCCTTGTGTAGCTCCCAAGAAGTT |
| | T |
| R13 | CCTGGACCATAACCATAAAATTTACCTAAAGATTGCAAGTAGTC |
| | GTAAACAGT |
| R14 | GAAGTCTTGTGCTGTGACAAAGAGCAGAATGGAGATAACAACTT |
| | TGAA |
| R15 | CCTCTTGAAGGTGATGATAAAGTAGTCAATGAAGATGGAGGGTA |
| | TGCAAC |
| R16 | TGTGTCAAATGGGCACAAATCCATCAATGACAAAACGTCTGTAT |
| | CAG |
| R17 | ATAATTCAACTCCTTTGAAACCCTGAGCGTTCAACCTTGCAGTA |
| | ATAG |
| R18 | GAGGAAAATTAGCAGCAATGAAAGTAGCCTGAACAGAATCTCCA |
| | GTT |
| R19 | GTTGAATCTTCAAAAGCTCTACACAAACCGTGGTGCATTGTATT |
| | GTTT |
| R20 | GCTGTTGGAGACTCTGATATAATGACCATCTCTTGTCTAGGCAA |
| | TGG |
| R21 | CAAAGAAGAAGGTGGAGGATTTTTGTCAGCCAACAAAGCAGAGT |
| | AG |
| R22 | AAACCAGTAGTGAAGTTCTTTGCAGATGCAATGACTCTTTCTTG |
| | ACC |
| R23 | TGATGCTCTAACGAAAGGCAATGATTCAGGATCTGTGTAGTCTC |
| | TC |
| R24 | AACAATGAAGCATACCTCTGGTAAAACTTTATTCCTGAGTTGAC |
| | CATTTCTT |
| R25 | GTTCACCAAAAGCTGTTAAATGGTCAACACCCAAAACATAGTTG |
| | TAATCTTTA |
| R26 | ATGAACTCGAAGTCTGGTGCGTACCATGTAGCAGATGTCTTAAT |
| | CTT |
| R27 | TGTTAAAACAGCAGATATAGCAGCAGCTTTTCCAGCAGTAGGGA |
| | A |
| R28 | TCTAGCTCCATGCCTAGATAAAACTTGTGCAAAAGTTAACCTAC |
| | ATCCA |
| R29 | GATGGAACTGAAGGTGAAATCTCAGATGGAACAGAGAAGAATGG |
| | TGA |
| R30 | GTATTGTCCCCAAGTATGAGTAGTTTTTTGGTCACATTGATAAC |
| | CTAACTCTG |

Step-two involved assembly of the five fragments into 2 longer fragments. Fragments 1, 2, and 3 and fragments 4 and 5 were combined, respectively, and designated fragments 123 and 45. Each 100 μl PCR reaction mixture (123 and 45) contained 5 μl of each of the gel-purified PCR fragment (reaction 123 contained fragments 1, 2, and 3; reaction 45 contained fragments 4 and 5), 10 μM forward primer, 10 μM reverse primer, 1× Failsafe Premix F (Epicentre Technologies, Madison, Wis.), and 5 U PFU Turbo Polymerase (Stratagene). The thermocycling program included one cycle of 94° C. (2 min), 10 cycles of 94° C. (30 sec), 50° C. (1 min), and 72° C. (45 sec +3 sec/cycle), 5 cycles of 94° C. (2 min), 55° C. (1 min), and 72° C. (65 sec +3 sec/cycle), 5 cycles of 94° C. (30 sec), 55° C. (1 min), and 72° C. (90 sec), and 20 cycles of 94° C. (30 sec), 60° C. (1 min), and 72° C. (90 sec) followed a final extension of 72° C. (10 min) and an indefinite 4° C. hold. PCR products were visualized and purified as stated above.

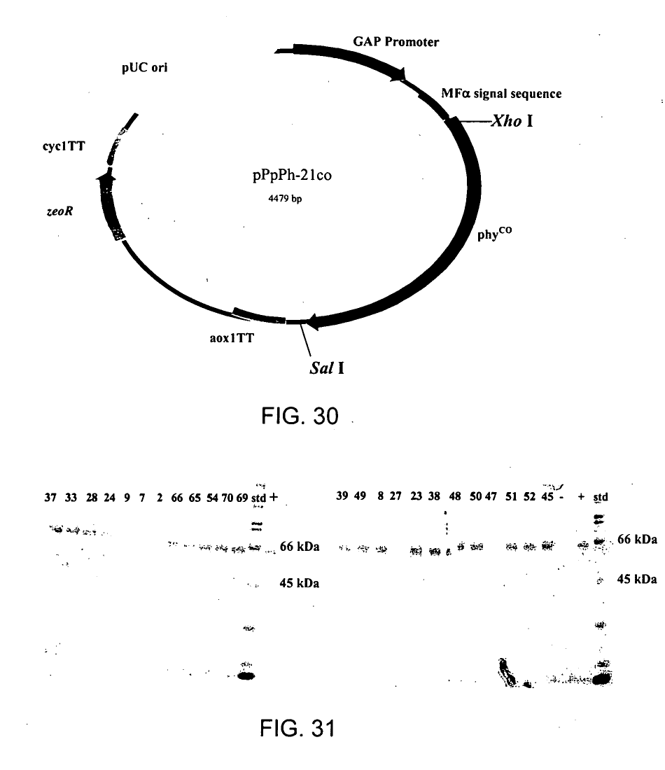

Step-three involved the final assembly of the full-length phy^{CO} gene by combining fragments 123 and 45. Each 100 μl PCR reaction mixture contained 2 μl gel purified PCR products 123 and 45, 1× Failsafe Premix F (Epicentre Technologies), 10 μM primer F1, 10 μM primer R1, and 5 U PFU Turbo Polymerase (Stratagene). The thermocycling program included one cycle of 94° C. (2 min) and 40 cycles of 94° C. (30 sec), 62° C. (1 min) and 72° C. (90 sec) followed by a final extension of 72° C. (10 min) and an indefinite hold at 4° C. The final assembly PCR product of the full-length phy^{CO} gene was visualized and purified as stated above. The phy^{CO} PCR product was digested with Xho I and Sal I and ligated into Xho I-Sal I digested pGAPZ to create plasmid pPpPh-21co. In this plasmid the sequence of the alpha factor secretion signal of Saccharomyces cerevisiae is fused in-frame to the 21st codon of the phy^{CO} gene and expression is driven by the constitutive GAP promoter (P_{GAP}) of P. pastoris (FIG. 30). The DNA sequence of the P_{GAP}-MFα-phy^{CO} expression cassette was confirmed as stated above.

Table 22 is the DNA sequence of the synthetic, codon-optimized, codon changed phytase gene sequence: the first shaded sequence, CTCGAG, is the Xho I restriction endonuclease recognition sequence, the second shaded sequence, AAGAGA, is the sequence that codes for the KEX2 dipeptide cleavage site, the third shaded sequence, TCT, is codon 21, the fourth shaded sequence, TAA, is the stop codon, and the fifth shaded sequence, GTCGAC, is the Sal I restriction endonuclease recognition sequence.

TABLE 22
The DNA sequence (SEQ ID NO. 3) of the synthetic. codon-optimized. codon
changed phytase gene sequence

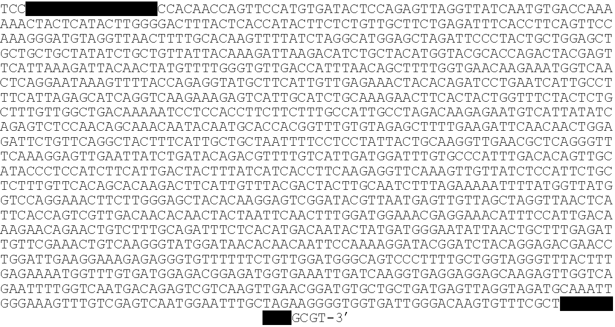

Table 23 is the deduced amino acid sequence of the gene of Table 22:

TABLE 23

The deduced amino acid sequence (SEQ ID NO. 4) of
the gene of Table 22

SPQPVPCDTPELGYQCDQKTTHTWGLYSPYFSVASEISPSVPKGCRLTFA
QVLSRHGARFPTAGAAAAISAVITKIKTSATWYAPDYEFIKDYNYVLGVD
HLTAFGEQEMVNSGIKFYQRYASLLRNYTDPESLPFIRASGQERVIASAK
NFTTGFYSALLADKNPPPSSLPLPRQENVIISESPTANNTMHHGLCRAFE
DSTTGDSVQATFIAANFPPITARLNAQGFKGVELSDTDVLSLMDLCPFDT
VAYPPSSLTTLSSPSRGSKLLSPFCSLFTAQDFIVYDYLQSLEKFYGYGP
GNFLGATQGVGYVNELLARLTHSPVVDNTTTNSTLDGNEETFPLTKNRTV
FADFSHDNTMMGILTALRLFETVKGMDNTTIPKGYGSTGDEPGLKEREGV
FSVGWAVPFAGRVYFEKMVCDGDGDGEIDQGEEEQELVRILVNDRVVKLN
GCAADELGRCKLGKFVESMEFARRGGDWDKCFA

The amino acid changes are listed below. The numbering is according to sequence of mature protein where codon 21 corresponds to amino acid 1.

Nomenclature: wildtype amino acid, amino acid number in linear sequence, changed amino acid.

1. Q27L

2. K66A

3. G293E

4. F30Y

5. P34A

6. S43K

7. L74I

8. F87Y

9. D127N

10. M178N

11. L195T

12. T284I

13. R322H

14. D359T

15. E274K

16. K402S

17. E454A

P. pastoris transformation and culture-tube expression of recombinant phy^{CO} phytase. Cells of P. pastoris strains KM71H were transformed by electroporation with 5 μg of Avr II linearized pPpPh-21co according to the method of Sears et al. (40). Immediately following electroporation cells were plated on YPD agar containing 100 μg/mL and 250 μg/mL zeocin™ (YPDZ₁₀₀ and YPDZ₂₅₀) and incubated for 2 days at 30° C. Resultant colonies were re-streaked onto YPDZ₁₀₀ and YPDZ₂₅₀, respectively, and grown for 2 days at 30° C. to confirm their phenotype. Zeocin™-resistant (zeo^{R}) transformants from the YPDZ₂₅₀ selection plate were inoculated into 14 mL Falcon tubes containing 1 mL YPD broth and grown overnight at 30° C. and 300 rpm. After growing overnight, biomass was removed by centrifugation and an aliquot of each sample was assayed for phytase activity using the microtiter plate method described in Example 1, with some minor modifications. The most notable modification was that each sample served as its own control. Controls consisted of addition of TCA to each sample prior to phytate addition, followed by incubation at 37° C. for one hour. Based on the results of the phytase activity assay, two transformants were chosen for further study, PpPh-21co-48 and PpPh-21co-69. Aliquots of a subset of samples were also analyzed for rPhy^{CO} production by SDS-PAGE.

Analytical methods. 10% NuPAGE® Novex Bis-Tris [Bis(2-hydroxyethyl) imino-tris (hydroxymethyl) methane-HCl] Pre-Cast Gels (Invitrogen) were used for separating proteins present in spent culture broth supernatant according to manufacture's instructions. Proteins in NuPAGE gels were visualized by staining with GelCode Blue (Pierce Biotechnology, Rockford, Ill.).

Expression of rPhy^{CO} under fermentative conditions. Transformants PpPh-21co-48 and PpPh-21co-69 were chosen to test for rPhy^{CO} production under fermentative conditions. A 300-mL seed culture of each transformant was grown in food-grade YPD medium [1.0% (w/v) FNI 200 yeast extract (Lallemand), 2.0% (w/v) Hy-Soy peptone (Quest International), 2.0% (w/v) dextrose] for 24 h at 30° C., 200 rpm. Each seed culture was used to inoculate a 14-L fermentor (New Brunswick Scientific Co.) containing 8 L of Basal Salt Medium with 40 g/L dextrose, 400 mg/L L-histidine, 0.9 mg/L biotin, and 1×PTM1 trace element solution (39). The fermentor temperature was controlled at 30° C. and dissolved oxygen maintained at 20% via agitation manipulations. The pH was regulated at 5.5 with 100% ammonium hydroxide, which also served as a nitrogen source. Aeration was maintained at ca. 1 vvm throughout the fermentation. A 5% (w/v) solution of Struktol J673 defoamer (Qemi International) was added as needed to control foaming. Upon depletion of dextrose in the fermentor, a feed containing 50% (w/v) Cerelose (dextrose), 0.4-0.7 g/L L-histidine, 2.1 mg/L biotin, and 6×PTM1 trace element solution was initiated at 3 g/L/hr dextrose. The feed rate was increased over a period of 24 hr to a maximum of 7 g/L/hr. During feed, dissolved oxygen was maintained at >10% first with agitation manipulations until maximum agitation had been achieved, followed by feed regulations. Cultures were sampled daily to monitor cell density and rPhy^{CO} production.

B. Results and Discussion

Synthesis and cloning of the codon-optimized phytase gene into pGAPZ and transformation of P. pastoris. The aim of this study was to increase expression of soluble and active phytase protein in P. pastoris. Using Pichia codon usage tables as a reference, each amino acid (AA) of the KPF0019 deduced AA sequence was converted into the corresponding codon preferentially used by P. pastoris. This resulted in the generation of an artificial phytase gene sequence where 67% of the original KPF-phy gene codons had been changed. The resultant phy^{CO} gene was inserted into the Xho I-Sal I sites of the P. pastoris constitutive expression vector pGAPZ, forming pPpPh-21co (FIG. 30). This plasmid contains an N-terminal translational fusion of the alpha factor secretion signal (MFα) (plus the Pro-region), a KEX2 protease recognition sequence ending with Lys-Arg, and the KPF-phy gene sequence starting at codon 21 (bp 127)

(42). The constitutive glyceraldehyde-3-phosphate dehydrogenase promoter (P_{GAP}) drives expression of the fusion in P. pastoris. In the endoplasmic reticulum the first 19 amino acids of the MFα peptide are cleaved by signal peptidase and in the Golgi the KEX2 protease cleaves the MFα Pro-region-phytase fusion at the Pro-region after the Lys-Arg dipepetide. This cleavage results in a mature, recombinant phytase protein beginning with serine. In order to increase homologous recombination efficiency, plasmid pPpPh-21co was linearized with Avr II prior to electroporation into P. pastoris KM71H. Since plasmid pPpPh-21co lacks a yeast origin of replication, it cannot autonomously replicate in P. pastoris. Therefore, the recovery of zeo^{R} transformants denotes the integration of at least one copy of the linearized plasmid into the chromosome of P. pastoris and homologous recombination occurs within the upstream 5′ sequence of the GAP promoter region of the P. pastoris chromosome. Research has shown that increasing the zeocin™ concentration in the selection media gives rise to transformants that have undergone multiple integration events and therefore contain multiple copies of the target gene of interest. To increase the phy^{CO} gene copy and potentially rPhy^{CO} production, transformants were selected on 250 μg/mL zeocin™. Zeo^{R} and phytase enzyme activity confirmed the presence of the integrated plasmid.

Screening of zeo^{R} transformants and culture-tube expression. P. pastoris strain KM71H was transformed with pPpPh-21co and 67 transformants were isolated from the YPDZ₂₅₀ plates and 499 transformants from the YPDZ₁₀₀ plates. The 67 zeo^{R} transformants isolated on YPDZ₂₅₀ and three transformants isolated on YPDZ₁₀₀ were examined for phytase activity. The negative control consisted of P. pastoris GS115 transformed with pGAPZ, which does not contain the KPF-phy gene and the positive control consisted on GS115 transformed with pPpPh23-G1, which contains the native KPF-phy gene (Table 24).

TABLE 24
Phytase activity in culture broths of P. pastoris
pPpPh-21co transformants

| | Phytase |
|---|---|
| | activity¹ |
| Transformant | (μmol/min/ml) |
| 1 | 0.995 |
| 2 | 1.734 |
| 3 | 1.622 |
| 4 | 1.755 |
| 5 | 1.675 |
| 6 | 1.605 |
| 7 | 1.801 |
| 8* | 2.387 |
| 9 | 1.825 |
| 10 | 1.626 |
| 11 | 1.247 |
| 12 | 1.214 |
| 13 | 1.410 |
| 14* | 0.09 |
| 15 | 1.291 |
| 16 | 1.636 |
| 17* | −0.129 |
| 18 | 1.716 |
| 19 | 1.741 |
| 20 | 1.771 |
| 21 | 1.374 |
| 22 | 1.864 |
| 23 | 1.966 |
| 24 | 1.876 |
| 25 | 1.749 |
| 26 | 1.538 |
| 27 | 1.900 |
| 28 | 1.827 |
| 29 | 1.699 |
| 30 | 1.578 |
| 31 | 1.566 |
| 32 | 1.551 |
| 33* | 1.845 |
| 34 | 1.725 |
| 36 | 1.707 |
| 37 | 1.866 |
| 38 | 1.995 |
| 39* | 2.725 |
| 40 | 1.692 |
| 41 | 1.546 |
| 42 | 1.669 |
| 43 | 1.441 |
| 44 | 1.776 |
| 45 | 1.884 |
| 46* | −0.069 |
| 47 | 2.105 |
| 48 | 1.950 |
| 49 | 2.064 |
| 50 | 2.092 |
| 51 | 1.969 |
| 52 | 1.960 |
| 53 | 1.313 |
| 54* | 2.587 |
| 55 | 1.893 |
| 56 | 1.456 |
| 57 | 1.697 |
| 58 | 1.658 |
| 59 | 1.483 |
| 60 | 1.519 |
| 61 | 1.626 |
| 62 | 1.724 |
| 63 | 1.852 |
| 64* | 0.678 |
| 65* | 2.474 |
| 66 | 2.022 |
| 67 | 1.691 |
| 68 | 1.206 |
| 69 | 1.992 |
| 70 | 2.080 |
| PpPh23-G1* | 1.561 |
| G-pKB* | −0.141 |

¹Data presented are the result of one experiment.
*Data presented represent the average of two replicates of the same experimental sample.

Transformants 17 and 46 showed no phytase activity, whereas transformants 1, 11, 12, 13, 14, 15, 21, 30, 31, 32, 41, 42, 43, 53, 56, 59, 60, and 64 displayed phytase activity lower than that of the positive control (Table 24). No activity was present in the negative controls. The lower levels of expression seen in these transformants were unexpected since research shows that codon-optimization enhances expression levels. In addition, all transformants tested were selected on a high concentration of zeocin™, indicating multiple-copy integration events had occurred thus increasing the gene copy number and potentially increasing rPhy^{CO} expression levels. It is unclear why these transformants do not show elevated phytase activity. The remaining transformants showed phytase activity equal to or above that of the positive control, with transformants 8, 23, 27, 38, 39, 45, 47, 48, 49, 50, 51, 52, 54, 65, 66, 69, and 70 showing the highest levels (Table 24). Table 25 shows the results of a repeat of the culture-tube expression study on transformants that showed the highest phytase activity levels. Transformants 48 and 69 showed the highest phytase activity as compared to the control, displaying 1.4- and 1.5-fold increases, respectively. These two transformants were chosen for further study and designated PpPh-21 co-48 and PpPh-21 co-69.

TABLE 25
Phytase activity in culture broth supernatant of high-
producing P. pastoris pPpPh-21co transformants

| | Phytase activity |
|---|---|
| Transformant | μmol/min/ml¹ |
| 8 | 3.022 |
| 23 | 3.052 |
| 27 | −0.175² |
| 38 | 3.154 |
| 39 | 3.000 |
| 45 | 3.322 |
| 47 | 0.208² |
| 48 | 3.605 |
| 49 | 2.741 |
| 50 | 2.641 |
| 51 | 2.571 |
| 52 | 2.710 |
| 54 | 3.414 |
| 65 | 3.074 |
| 66 | 3.289 |
| 69 | 3.441 |
| 70 | 3.093 |
| PpPh23-G1 | 2.453 |
| G-pKB | −0.148 |

¹Data are the result of one experiment.
²Overnight cultures of these transformants did not grow well.

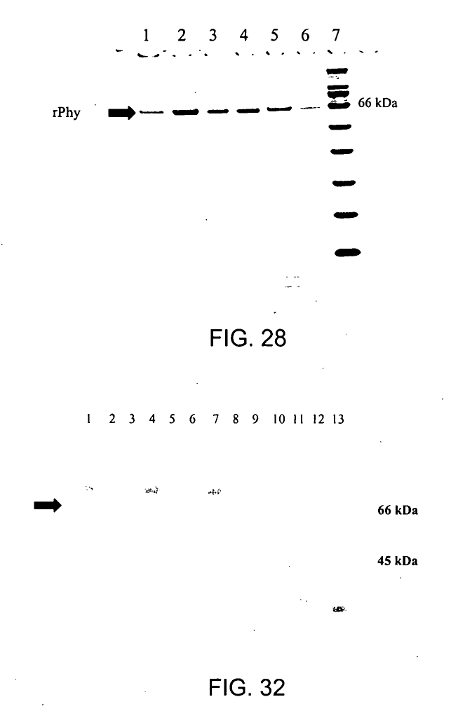

Production of rPhy^{CO} by pPpPh-21co transformants. Culture supernatants from a subset of transformants were examined for protein production by SDS-PAGE. All transformants appear to produce significant levels of rPhy^{CO} except for transformants 27 and 47 (FIG. 31). Each transformant's supernatant contained one predominant band in the acceptable molecular weight range for rPhy^{CO} whereas the negative control supernatant did not. This provides strong evidence that this protein band is rPhy^{CO}. The result also suggests the KEX2 protease correctly processed the MFα secretion signal peptide-Pro region.

Production of rPhy^{CO} by fermentation. Although heterologous proteins can be expressed well in P. pastoris shake-flask cultures, expression levels are typically low when compared to fermentative cultures. One reason is that only in the controlled environment of a fermentor is it possible to grow this organism to high cell density (OD₆₀₀ unit 500) (38). Especially for secreted proteins, the concentration of product in the culture medium is roughly proportional to the cell density in the fermentor. Because of these reasons, we decided to examine rPhy^{CO} production by transformants PpPh-21co-48 and PpPh-21co-69 under fermentative conditions. The fermentation process was run in fed-batch mode. Dextrose served as the sole carbon source and was maintained at a limited (>0.5%) concentration in the culture broth once initial dextrose was consumed. Cultures were sampled ca. every 24 hr and were fractioned into biomass and supernatants for SDS-PAGE analysis and phytase activity assay. SDS-PAGE confirmed the accumulation of rPhy^{CO} as the major protein secreted by both PpPh-21co-48 and PpPh-21co-69 (FIG. 32, arrow). In FIG. 32, lanes 1-5 are fermentation samples of rPhy^{CO} produced by strain PpPh-21co-69; lane 1 is a 111.5 hr fermentation sample (1 μl); lane 2 is an 85 hr fermentation sample (1 μl); lane 3 is a 61 hr fermentation sample (1 μl); lane 4 is a 36.5 hr fermentation sample (5.0 μl); lane 5 is a 15.5 hr fermentation sample (5.0 μl); lane 6 is a culture-tube sample of rPhy^{CO} produced from PpPh-21co-69 (5 μl); lanes 7-11 are fermentation samples of rPhy^{CO} produced by strain PpPh-21co-48; lane 7 is a 111.5 hr fermentation sample (1 μl); lane 8 is an 85 hr fermentation sample (1 μl); lane 9 is a 61 hr fermentation sample (1 μl); lane 10 is a 36.5 hr fermentation sample (5.0 μl); lane 11 is a 15.5 hr fermentation sample (5.0 μl); lane 12 is a culture-tube sample of rPhy^{CO} produced from PpPh-21co-48 (5 μl); and lane 13 is a protein MW standard. The analysis also showed that fermentative growth of both transformants increased rPhy^{CO} production approximately 3- to 5-fold over culture-tube production (FIG. 32, lanes 6 versus lanes 1-5, and lane 12 versus lanes 7-11). At 85 hours post-inoculation (HPI) a maximum phytase activity of 17 U/mL was reached (Table 26). This activity is slightly lower than that produced by the KPF-phy transformants under fermentative conditions, which is unexpected as both Pp-Ph-21co-48 and Pp-Ph-21co-69 grew to higher cell densities than did the KPF-phy transformant under similar conditions. There is no immediate explanation for the lower activity, however, it is still 8-fold higher than in the typical culture-tube supernatant (Table 24). Phytase production essentially ceased after 85 HPI, a phenomenon that has been observed with other P. pastoris strains run under fed-batch conditions.

TABLE 26
Phytase activity and protein concentration in fermentation
broth of P. pastoris pPpPh-21co transformants

| | | Phytase | |
|---|---|---|---|
| | Hours Post- | Activity² | Protein² |
| Transformant¹ | Inoculation | μmol/min/ml) | (mg/ml) |
| PpPh-21co-48 | HPI 15.5 | 0.08 | 0.03 |
| | HPI 36.5 | 5.11 | 1.23 |
| | HPI 61 | 9.28 | 2.48 |
| | HPI 85 | 17.32 | 3.30 |
| | HPI 111.5 | 16.22 | 3.73 |
| PpPh-21co-69 | HPI 15.5 | 0.88 | 0.07 |
| | HPI 36.5 | 7.44 | 1.56 |
| | HPI 61 | 13.82 | 3.02 |
| | HPI 85 | 17.47 | 3.82 |
| | HPI 111.5 | 17.26 | 4.28 |
| ³G-pKB | Positive | 2.15 | 0.38 |
| | control | | |
| ⁴G-pKB | Negative | 0.00 | 0.18 |
| | control | | |

¹Generated by transformation of P. pastoris strain KM71H with pPpPh-21co.
²Data presented are the results of one experiment.
³Postitive control generated by transformation of P. pastoris strain GS115 with pGAPZ.
⁴ Negative control generated by transformation of P. pastoris strain GS115 with pGAPZ.

Summary. Although the ultimate level of expression of a protein is largely dependent on its inherent properties, the expression level can be optimized by adjusting one or more parameters, such as changing gene dosage, optimizing the mRNA 5′UTR, using preferred codons, and adjusting medium and growth conditions. In this report we focused on several of these parameters with the aim of increasing rPhy production in P. pastoris. First a codon-optimized phytase gene was designed and synthesized for expression in P. pastoris. The gene was then transformed and transformants selected based on increased gene dosage. Finally, using fermentation, growth media and conditions were adjusted to increase rPhy^{CO} production. As a result we were able to increase rPhy^{CO} protein production significantly. When strains PpPh-21co-48 and PpPh-21co-69 were grown under fermentative conditions protein production increased approximately 4-fold as compared to growth under culture-tube conditions (culture tube total protein data not shown). It is important to note that the fermentation conditions were an initial effort to grow the strains and are not optimized. Therefore it is likely expression can be increased even further through fermentation optimization.

The foregoing description and drawings comprise illustrative embodiments of the present inventions. The foregoing embodiments and the methods described herein may vary based on the ability, experience, and preference of those skilled in the art. Merely listing the steps of the method in a certain order does not constitute any limitation on the order of the steps of the method. The foregoing description and drawings merely explain and illustrate the invention, and the invention is not limited thereto, except insofar as the claims are so limited. Those skilled in the art who have the disclosure before them will be able to make modifications and variations therein without departing from the scope of the invention.

### REFERENCES

1. Urbano, G., Lopez-Jurado, M., Aranda, P., Vidal-Valerde, C., Tenorio, E., and Porres, J. (2000) The role of phytic acaid in legumes: antinutrient or beneficial function? J. Physiol. Biochem. 56(3): 283-294.
2. Saylor, W. W. (2000) Reduce phosphorus in diets to protect the environment.
3. Dvorakova, J. (1998) Phytase: Sources, Preparation and Exploitation. Folia Microbiol. 43(4): 323-338.
4. Codon usage tabulated from the international DNA sequence databases: status for the year 2000. Nakamura, Y., Gojobori, T. and Ikemura, T. (2000) Nucl. Acids Res. 28, 292.
5. Sandberg, A.-S. and Ahderinne, R. (1986) HPLC Method for determination of Inositol Tri-, Tetra-, Penta-, and Hexaphosphates in Foods and Intestinal Contents. J. Food Sci. 51(3): 547-550.
6. Karlin, S. and Altschul, S. F. (1993) Application and statistics for multiple high-scoring segments in molecular sequences. Proc. Natl. Acad. Sci. 90: 5873-5877.
7. Ichibiki (1995) Phytase and its preparation. Japan Pat. 7059562.
8. Adeola, O., B. V. Lawrence, A. L. Sutton, and T. R. Cline. 1995. Phytase-induced changes in mineral utilization in zinc-supplemented diets for pigs. J. Anim. Sci. 73:3384:3391.
9. Current Protocols in Molecular Biology, Chapter 2, Eds., Ausubel, et. al., Greene Publishing and Wiley-Interscience, New York (2002).
10. Saghai-Maroof, M. A., K. M. Soliman, R. A. Jorgensen, and R. W. Allard. 1984. Ribosomal DNA spacer-length polymorphisms in barley: mendelian inheritance, chromosomal location, and population dynamics. Proc Natl Acad Sci. 81:8014-8.
11. Sambrook, J., E. F. Fritsch, and T. Maniatis. 1989. Molecular cloning: a laboratory manual, 2nd ed. Cold Spring Harbor Laboratory Press, Cold Spring Harbor, N.Y.
12. Stahl, C. H., Y. M. Han, K. R. Roheker, W. A. House and X. G. Lei. 1999. Phytase improves iron bioavailibility for hemoglobin synthesis in young pigs. J. Anim. Sci. 77:2135-2142.
13. Vohra, A. and Satyanarayana, T (2003) Phytases: Microbial Sources, Production, Purification, and Potential Biotechnological Applications. Crit. Rev. Biotech. 23 (1): 29-60.
14. Ebune, A., Al-Asheh, S. and Duvnjak, Z. (1995). Effects of Phosphate, Surfactants and Glucose on Phytase Production and Hydrolysis of Phytic Acid in Canola Meal by Aspergillus ficuum during Solid-State Fermentation. Bioresource Technology 54: 241-247.
15. Shieh, T. R. and Ware, J. H. (1968) Survey of microorganisms for the production of extracellular phytase. Appl. Microbiol. 169 (9): 1348-1351.
16. Al-Asheh, S and Duvnjak, Z (1995) Phytase production and decrease of phytic content in canola meal by Aspergillus carbonarius in solid-state fermentation. World J. Microbiol. Biotechnol. 11:228-231.
17. Wyss, M., Pasamontes, L., Remy, R., Kohler, J., Kusznir, E., Gadient, M., Muller, F., G. M. van Loon, A. (1998) Comparison of the Thermostaiblity Properties of Three Acid Phosphatases from Molds: Aspergillus fumigatus Phytase, A. niger Phytase, and A. niger pH 2.5 Acid Phosphatase. Applied and Env. Micro. 64: 4446-4451.
18. Finase phytase technical information by Rohm Enzyme Finland (1999).
19. Phytase: Interesting facts about Natuphos from Research and Practical Experience Edition 29 by BASF (1993).
20. Ronozyme P: A new phytase for animal feed (2004) by Roche Vitamins.
21. Simon, O. and Igbasan, F. (2002) In vitro properties of phytase from various microbial origins. International Journal of Food Science and Technology, 37, 813-822.
22. Lolas, G. M., and P. Markakis. 1977. Phytate of navy beans. J. Food Sci. 42:1094-1097.
23. Cromwell, G. L. and R. D. Coffey. 1991. Phosphorus, a key essential nutrient, yet a possible major pollutant—its central role in animal nutrition, p. 134-145. In T. P. Lyons (ed.), Biotechnology in the feed industry. Alltech Technical Publications. Nicholasville, Ky.
24. Lei, X. G., P. K. Ku, E. R. Miller and M. T. Yokoyana. 1993. Supplementing corn-soybean meal diets with microbial phytase linearly improves phytate phosphorus utilization by weanling pigs. J. Anim. Sci. 71:3359-3367.
25. Cromwell, G. L., R. D. Coffey, G. R. Parker, H. J. Monegue, and J. H. Randolf. 1995. Efficacy of a recombinant-derived phytase in improving the bioavailibility of phosphorus in corn-soybean meal diets for pigs. J. Animal Sci. 73:2000-2008.
26. O'Quinn, P. R., D. A. Knabe and E. J. Gregg. 1997. Efficacy of Natuphos in sorgum-based diets of finishing swine. J. Anim. Sci. 75:1299-1307.
27. Kerovuo, J. A Novel Phytase from Bacillus. 2000. Characterization and Production of the Enzyme. Danisco Cultor Innovation, Kantvik, Finland. Academic Dissertation.
28. Ausubel, et. al. Current Protocols in Molecular Biology, Eds. Greene Publishing and Wiley-Interscience, New York (2002).

29. Durand, H., M. Clanet and G. Tiraby. 1988. Genetic imrovement of Trichoderma reesei for large scale cellulase production. Enzyme Microb. Technol. 10: 341-346.
30. Goller, S. P., D. Schoisswohl, M. Baron, M. Parriche, and C. P. Kubicek. 1998. Role of endoproteolytic dibasic processing in maturation of secretory proteins in Trichoderma reesei. Appl. Enviro. Microbiol. 64: 3202-3208.
31. Walter, P. and A. E. Johnson. 1994. Signal sequence recognition and protein targeting to the endoplasmic reticulum. Ann. Rev. Cell Biol. 10: 87-119.
32. Henrique-Silva, F., S. El-Gogary, J. C. Carle-Urioste, E. Matheucci Jr., O. Crivellaro, and E. El-Dorry. 1996. Two regulatory regions controlling basal and cellulose-induced expression of the gene encoding cellobiohydrolase I of Trichoderma reesei are adjacent to its TATA box. Biochem. Biophys. Res. Comm. 228: 229-237.
33. Keranen, S. and M. Pettila. 1995. Production of recombinant proteins in the filamentous fungus Trichoderma reesei. Curr. Biol. 6: 534-537.
34. Nyyssonen, E., M. Pettila, A. Harkki, A. Saloheimo, J. K. C. Knowles, and S. Keranen. 1993. Efficient production of antibody fragments by the filamentous fungus Tricoderma reesei. Bio/Technology 11: 591-595.
35. Uusitalo, J. M., K. M. H. Nevalainen, A. M. Harkki, J. K. C. Knowles, and M. E. Penttila. 1991. Enzyme production by recombinant Trichoderma reesei strains. J. Biotechnol. 17: 35-50.
36. Sambrook, J., E. F. Fritsch, and T. Maniatis. 1989. Molecular cloning: a laboratory manual, 2nd ed. Cold Spring Harbor Laboratory Press, Cold Spring Harbor, N.Y.
37. Ilmen, M. M. L. Onnela, S. Klemsdal, S. Keranen, and M. Penttila. 1996. Functional analysis of the cellobiohydrolase I promoter of the filamentous fungus Trichoderma reesei. Mol. Gen. Genet. 253: 303-314.
38. Gleeson, M. A. and P. E. Sudbery. 1998. The methylotropic yeasts. Yeast 4:1-15.
39. Higgins, D. R., and Cregg, J. M. (1998) in Pichia protocols (Higgins, D. R., and Cregg, J. M., eds) Vol. 103, pp. 1-15, Humana Press, Inc., Totowa, N.J.
40. Sears, I. B., J. O'Connor, O. W. Rossanese, and B. S. Glick. 1998. A versatile set of vectors for constitutive and regulated gene expression in Pichia pastoris. Yeast. 14:783-790.
41. Sinclair, G. and F. Y. M. Choy. 2002. Synonomous codon usage and the expression of human glucocerebrosidase in the methylotropic yeast, Pichia pastoris. Prot. Exp. Pur. 26:96-105.
42. Sreekrishna, K. 1993. Strategies for optimizing protein expression and secretion in the methylotropic yeast Pichia pastoris. In Industrial Microorganisms: Basic and Applied Molecular Genetics, Chapter 16, p 119-126, R. H. Baltz, et al., Eds. American Society for Microbiology, Washington, D.C.
43. Pandey, A., G. Szakacs, C. R. Soccol, J. A. Rodriguez-Leon, and V. T. Soccol. 2001. Production, purification, and properties of microbial phytases. Bioresc. Technolo. 77: 203-214.

We claim:

1. A phytase excreted by an isolated fungal strain ATCC accession no. SD5361.

2. A phytase as defined in claim 1, wherein the phytase has a pH optimum of about 5.5.

3. A phytase as defined in claim 1, wherein the phytase has a temperature optimum of about 55° C.

4. A phytase as defined in claim 1, wherein the activity of the phytase retains at least 30% of its maximum activity over a temperature range from about 80° C. to about 100° C.

5. An isolated nucleic acid which encodes a phytase having an activity profile wherein the activity of the phytase retains at least 30% of its maximum activity over a temperature range from about 80° C. to about 100° C. and which is at least 74.9% identical to SEQ ID No. 1 wherein the % identity is determined using a sequence comparison algorithm or by visual inspection.

6. The isolated nucleic acid sequence of claim 5, wherein the sequence comparison algorithm is the AlignX alignment program of Vector NTI Suite 7.1 using a Gap penalty of 15, a Gap extension penalty of 6.66, and a Gap separation penalty range of 8.

7. The isolated nucleic acid sequence in claim 5, which is at least 85% identical to SEQ ID No. 1 wherein the % identity is determined using a sequence comparison algorithm.

8. The isolated nucleic acid sequence of claim 7, wherein the sequence comparison algorithm is the AlignX alignment program of Vector NTI Suite 7.1 using a Gap penalty of 15, a Gap extension penalty of 6.66, and a Gap separation penalty range of 8.

9. The isolated nucleic acid sequence as defined in claim 5, wherein the nucleic acid is a DNA molecule.

10. A plasmid comprising an isolated DNA molecule which encodes a phytase which retains at least 30% of its maximum activity over a temperature range from about 80° C. to about 100° C. and which is at least 70% identical to SEQ ID No. 1 wherein the % identity is determined using a sequence comparison algorithm or by visual inspection.

11. The isolated DNA molecule sequence of claim 10, wherein the sequence comparison algorithm is the AlignX alignment program of Vector NTI Suite 7.1 using a Gap penalty of 15, a Gap extension penalty of 6.66, and a Gap separation penalty range of 8.

12. The isolated DNA molecule sequence in claim 10, which is at least 85% identical to SEQ ID No. 1 wherein the % identity is determined using a sequence comparison algorithm or by visual inspection.

13. The isolated DNA molecule sequence of claim 12, wherein the sequence comparison algorithm is the AlignX alignment program of Vector NTI Suite 7.1 using a Gap penalty of 15, a Gap extension penalty of 6.66, and a Gap separation penalty range of 8.

14. A prokaryotic host cell transformed by the nucleic acid sequence as of claim 5.

15. A transformed host cell as defined in claim 14, wherein the prokaryotic host cell is E. coli.

16. A eukaryotic host cell transformed by the nucleic acid sequence of claim 5.

17. A transformed host cell as defined in claim 16, wherein the eukaryotic host cell is selected from the group consisting of Pichia sp. and Trichoderma sp.

18. A synthetic nucleic acid sequence comprising conversion of each amino acid of a deduced amino acid sequence of the nucleic acid of claim 5 into the corresponding codon preferentially used by a selected host cell to be transformed using the artificial nucleic acid sequence.

19. The synthetic nucleic acid sequence of claim 18 which is at least 70% identical to SEQ ID NO. 3 as determined by analysis with a sequence comparison algorithm or by visual inspection.

20. A method of producing a phytase, comprising the steps of:
(a) transforming a eukaryotic host cell with the nucleic acid of claim 5;
(b) growing the eukaryotic host cell under conditions effective for producing the phytase; and
(c) recovering the phytase.

21. An isolated nucleic acid which encodes a phytase having an activity profile wherein the activity of the phytase retains at least 30% of its maximum activity over a temperature range from about 80° C. to about 100° C. and which hybridizes to SEQ ID No. 1 under conditions of low stringency.

22. An isolated nucleic acid as defined in claim 20 wherein the hybridization is under conditions of high stringency.

23. An isolated phytase protein which retains at least 30% of its maximum activity over a temperature range from about 80° C. to about 100° C. and comprising an amino acid sequence that is at least 70% identical to SEQ ID NO. 2 as determined by analysis with a sequence comparison algorithm or by visual inspection.
